(19) United States
(12) Patent Application Publication
(10) Pub. No.: US 2025/0277272 A1
(43) Pub. Date: Sep. 4, 2025

(54) CANCER TREATMENTS AND METHODS OF SELECTING SAME

(71) Applicant: The General Hospital Corporation, Boston, MA (US)

(72) Inventor: Bin ZHENG, Charlestown, MA (US)

(73) Assignee: The General Hospital Corporation, Boston, MA (US)

(21) Appl. No.: 19/059,839

(22) Filed: Feb. 21, 2025

Related U.S. Application Data
(63) Continuation of application No. 18/323,922, filed on May 25, 2023, now abandoned.
Application No. 18/323,922 is a continuation of application No. 16/998,690, filed on Aug. 20, 2020, now abandoned.
Application No. 16/998,690 is a continuation of application No. 16/071,427, filed on Jul. 19, 2018, now abandoned.
(60) Provisional application No. 62/280,358, filed on Jan. 19, 2016.

(51) Int. Cl. C12Q 1/6886

(52) U.S. Cl. CPC C12Q 1/6886; C12Q 2600/106; C12Q 2600/156; C12Q 2600/158

(57) ABSTRACT

Provided herein are methods for treating cancer and for selecting a cancer treatment based on the expression of Stag2/3 proteins and/or the presence of mutations in the genes encoding Stag2/3 proteins.

### CROSS-REFERENCE TO RELATED APPLICATIONS

This application is a Continuation of U.S. patent application Ser. No. 18/323,922 filed May 25, 2023, which is a Continuation of U.S. patent application Ser. No. 16/998,690 filed Aug. 20, 2020, which is a Continuation of U.S. patent application Ser. No. 16/071,427 filed Jul. 19, 2018, which is a 35 U.S.C. § 371 National Phase Entry Application of International Application No. PCT/US2017/013150 filed Jan. 12, 2017, which designates the U.S. and which claims benefit under 35 U.S.C. § 119 (e) of U.S. Provisional Application No. 62/280,358 filed Jan. 19, 2016, the contents of each of which are incorporated herein by reference in their entireties.

### SEQUENCE LISTING

The instant application contains a Sequence Listing which has been submitted electronically in XML format and is hereby incorporated by reference in its entirety. Said XML copy, created on May 25, 2023, is named “030258-085911USC2_SL.xml” and is 36,183 bytes in size.

### FIELD OF THE INVENTION

The field of the invention relates to methods for the treatment of cancer.

### BACKGROUND

The processes involved in tumor growth, progression, and metastasis are mediated by signaling pathways that are activated in cancer cells. The ERK pathway plays an important role in regulating mammalian cell growth. Activation of the ERK pathway occurs through a cascade of phosphorylation events that begins with activation of Ras, which in turn leads to the recruitment and activation of Raf, a serine-threonine kinase. There are three isoforms of Raf: ARAF, BRAF, and CRAF, that activate the MEK-ERK cascade. Activated Raf then phosphorylates and activates MEK1/2, which in turn phosphorylates and activates ERK1/2. Activated ERK1/2 phosphorylates several downstream targets involved in a multitude of cellular events including cytoskeletal changes and transcriptional activation.

The ERK/MAPK pathway is one of the most important for cell proliferation, and it is believed that the ERK/MAPK pathway is frequently activated in many tumors. Ras genes, which are upstream of ERK1/2, are mutated in several cancers including colorectal, melanoma, breast and pancreatic tumors. High Ras activity is accompanied by elevated ERK activity in many human tumors. In addition, mutations of BRAF, a serine-threonine kinase of the Raf family, are associated with increased kinase activity.

### SUMMARY

The methods and treatments described herein are based, in part, on the discovery that loss-of-function Stag2 and/or Stag3 mutations or a decreased expression of Stag2/3 proteins is correlated with acquired resistance to BRAF inhibitors (BRAFi).

Accordingly, provided herein is a method of selecting a treatment for cancer, the method comprising: measuring the activity and/or expression levels of Stag 2 and/or Stag3 in a biological sample (e.g., a tumor sample) obtained from a subject having or suspected of having cancer, wherein if the activity and/or expression levels in the sample are substantially similar or increased compared to a reference, administration of a treatment comprising a BRAF inhibitor is selected, and wherein if the activity and/or expression levels in the sample are decreased compared to a reference, administration of a treatment is selected from the group consisting of: a PD-1 inhibitor, a PD-L1 inhibitor, an ERK inhibitor and a combination thereof.

In one embodiment of this aspect and all other aspects provided herein, the method further comprises a step of selecting a subject having a cancer that comprises a mutation in BRAF, Stag2 and/or Stag3.

In another embodiment of this aspect and all other aspects provided herein, the subject comprises an inherent resistance to BRAF inhibitors.

In one embodiment of this aspect and all other aspects provided herein, the subject comprises an acquired resistance to BRAF inhibitors.

In another embodiment of this aspect and all other aspects provided herein, the mutation in BRAF is Val600Glu (V600E) or Val600Lys (V600K).

In another embodiment of this aspect and all other aspects provided herein, the mutation in Stag2 is a loss-of-function mutation.

In another embodiment of this aspect and all other aspects provided herein, the loss-of-function mutation is Asp193Asn (D193N) or Lys1083Stop (K1083*).

In another embodiment of this aspect and all other aspects provided herein, the BRAF inhibitor is vemurafenib, dabrafenib, LGX818, sorafenib or PLX-4720.

In another embodiment of this aspect and all other aspects provided herein, the BRAF inhibitor is administered in combination with a MEK inhibitor.

In another embodiment of this aspect and all other aspects provided herein, the MEK inhibitor is trametinib, cobimetinib, MEK162, AZD6244, RO5126766, or GDC-0623.

In another embodiment of this aspect and all other aspects provided herein, the ERK inhibitor is SCH772982 or VTX11e.

In another embodiment of this aspect and all other aspects provided herein, the PD-1or PD-L1 inhibitor is nivolumab, pembrolizumab, pidilizumab, BMS-936559, or MPDL-3280A.

In another embodiment of this aspect and all other aspects provided herein, the reference is the activity and/or expression of Stag2 and/or Stag3 in a population of subjects known to have a cancer (e.g., tumor or lesion) that is responsive to a BRAF inhibitor or is in a phase of responsiveness to a BRAF inhibitor.

In another embodiment of this aspect and all other aspects provided herein, the cancer is selected from the group consisting of: non-Hodgkin lymphoma, colorectal cancer, melanoma, papillary thyroid carcinoma, non-small-cell lung carcinoma, and adenocarcinoma of the lung.

In another embodiment of this aspect and all other aspects provided herein, the method further comprises a step of administering the selected treatment to a subject.

In another embodiment of this aspect and all other aspects provided herein, the biological sample comprises a blood sample, a serum sample, a circulating tumor cell sample, a tumor biopsy, or a tissue sample.

In another embodiment of this aspect and all other aspects provided herein, the measuring step comprises contacting the biological sample with an antibody that specifically binds to Stag2 and/or Stag3.

In another embodiment of this aspect and all other aspects provided herein, the mutation in BRAF, Stag2 and/or Stag3 is identified by a DNA sequencing method.

In another embodiment of this aspect and all other aspects provided herein, the DNA sequencing method comprises real-time PCR. Sanger sequencing, pyrosequencing, a TH×ID BRAF mutation test, a COBAS® BRAF mutation test, and bidirectional direct sequencing.

Another aspect described herein relates to a method of monitoring a subject for the development of resistance to a BRAF inhibitor, the method comprising: measuring the activity and/or expression levels of Stag 2 and/or Stag3 in a sample obtained from a subject being treated with a BRAF inhibitor, wherein if the activity and/or expression levels are substantially similar or increased compared to the activity and/or expression levels prior to the onset of treatment with a BRAF inhibitor, the subject is determined to have a cancer that is sensitive to the BRAF inhibitor, and wherein if the activity and/or expression levels are decreased compared to the activity and/or expression levels prior to the onset of treatment with a BRAF inhibitor, the subject is determined to have a cancer that is resistant to or in the process of developing resistance to a BRAF inhibitor.

In one embodiment of this aspect and all other aspects provided herein, the subject determined to have a cancer that is resistant or is developing resistance to a BRAF inhibitor is treated with an ERK inhibitor, a PD-1 inhibitor and/or a PD-L1 inhibitor.

In another embodiment of this aspect and all other aspects provided herein, the ERK inhibitor is SCH772982 or VTX11e.

In another embodiment of this aspect and all other aspects provided herein, the PD-1or PD-L1 inhibitor is nivolumab, pembrolizumab, pidilizumab, BMS-936559, or MPDL-3280A.

In another embodiment of this aspect and all other aspects provided herein, the BRAF inhibitor is vemurafenib, dabrafenib, LGX818, sorafenib or PLX-4720.

In another embodiment of this aspect and all other aspects provided herein, the BRAF inhibitor is administered in combination with a MEK inhibitor.

In another embodiment of this aspect and all other aspects provided herein, the MEK inhibitor is trametinib, cobimetinib, MEK162, AZD6244, RO5126766, and GDC-0623.

In another embodiment of this aspect and all other aspects provided herein, the method further comprises a step of selecting a subject that has a cancer comprising a mutation in BRAF, Stag2 and/or Stag3.

In another embodiment of this aspect and all other aspects provided herein, the subject comprises an inherent resistance to BRAF inhibitors.

In one embodiment of this aspect and all other aspects provided herein, the subject comprises an acquired resistance to BRAF inhibitors.

In another embodiment of this aspect and all other aspects provided herein, the mutation in BRAF is Val600Glu (V600E) or Val600Lys (V600K).

In another embodiment of this aspect and all other aspects provided herein, the mutation in Stag2 is a loss-of-function mutation.

In another embodiment of this aspect and all other aspects provided herein, the loss-of-function mutation is Asp193Asn (D193N) or Lys1083Stop (K1083*).

In another embodiment of this aspect and all other aspects provided herein, the subject was diagnosed with a cancer selected from the group consisting of: non-Hodgkin lymphoma, colorectal cancer, melanoma, papillary thyroid carcinoma, non-small-cell lung carcinoma, and adenocarcinoma of the lung.

In another embodiment of this aspect and all other aspects provided herein, the biological sample comprises a blood sample, a serum sample, a circulating tumor cell sample, a tumor biopsy, or a tissue sample.

In another embodiment of this aspect and all other aspects provided herein, the measuring step comprises contacting the biological sample with an antibody that specifically binds to Stag2 and/or Stag3.

In another embodiment of this aspect and all other aspects provided herein, the mutation in BRAF, Stag2 and/or Stag3 is identified by a DNA sequencing method.

In another embodiment of this aspect and all other aspects provided herein, the DNA sequencing method comprises real-time PCR, Sanger sequencing, pyrosequencing, a TH×ID BRAF mutation test, a COBAS® BRAF mutation test, and bidirectional direct sequencing.

### BRIEF DESCRIPTION OF THE FIGURES

FIGS. 1A-1C. Decreased expression of Stag2 and Stag3 in BRAFi-resistant melanoma tumors and cell lines. FIG. 1A, Detection of STAG2 mutation in a post-relapse biopsy from a melanoma patient who relapsed from vemurafenib treatment. FIG. 1A discloses SEQ ID NOs: 23 and 24, respectively, in order of appearance. FIG. 1B, Changes of Stag2 and Stag3 protein levels in a panel of melanoma BRAFi-resistant cell lines and their parental BRAFi-sensitive counterparts. P: parental; BR: BRAFi resistant. BMR: BRAFi/MEKi double resistant. FIG. 1C, Immunohistochemical analyses of Stag2 and Stag3 in pairs of pre-treatment and post-relapse tumor samples from patients treated with BRAFi monotherapy or BRAFi/MEKi combination therapy.

FIGS. 2A-2H. Knock-down of STAG2 or STAG3 decreases BRAFi sensitivity in BRAF-mutant melanoma cells. FIG. 2A, Viability of A375 cells expressing cither a scrambled control shRNA or a STAG2-specific shRNA (shSTAG2 #23), after treatment with varying concentrations of dabrafenib (Dab) for 3 d. Experiment was performed three times. Data are mean±s.e.m. shSTAG2 #23 sequence: CCGGGCAAGCAGTCTTCAGGTTAAACTCGAGTTTAACCTGAAGACTGCTTGCTTT TTTG; SEQ ID NO. 1) scramble control shRNA sequence: (CCTAAGGTTAAGTCGCCCTCGCTCGAGCGAGGGCGACTTAACCTTAGG: SEQ ID NO. 2) FIG. 2B, Representative Western blot analysis of A375 cells that were treated with dabrafenib for 2 hr. Experiment was performed three times. FIG. 2C, Viability of SKMEL28 cells expressing an inducible STAG2-specific shRNA (shStag2 #60; TTAATGCTAAGATTTAGTG: SEQ ID NO. 3) and cultured in the presence (+Dox) or absence (−Dox) of doxycycline after treatment with varying concentrations of vemurafenib (VEM) for 3 d. Experiment was performed three times. FIG. 2D, Representative Western blot analysis of SKMEL30 cells that were treated with vemurafenib for 2 hr. FIG. 2E, Viability of A375 cells expressing either a scrambled control shRNA or a STAG3-specific shRNA (shSTAG3 #96), after treatment with varying concentrations of dabrafenib for 3 days. Experiment was performed 3 times. Data are mean±s.e.m. FIG. 2F, Representative Western blot analysis of A375 cells expressing either scrambled shRNA or shSTAG3 #96 (shSTAG3 #96 sequence: CCGGCGTGATTTCCTTAGGCCACTTCTCGAGAAGTGGCCTAAGGAAATCACGTTTTTTG; SEQ ID NO. 4) that were treated with vemurafenib for 2 hr. Experiment was performed three times. Data are presented as mean±s.e.m. FIG. 2G, Representative Western blot analysis of SKMEL30 cells expressing either a scrambled control shRNA or shSTAG2 #23, after treatment with varying concentrations of trametinib (Tra) for 2 h. Experiment was performed three times. FIG. 2H, Representative Western blot analysis of WM902-BR cells stably expressing a control vector or a vector expressing Flag-tagged wild-type STAG2 (WT). STAG2Lys1083* (K1083*) or STAG2Asp193Asn (D193N), after treatment with DMSO (−) or 3 μM vemurafenib (+) for 2 h. Experiment was performed three times.

FIGS. 3A-3F. Knockdown of Stag2 or Stag3 impairs the effects of vemurafenib on inhibiting melanoma xenograft tumor growth in vivo. FIG. 3A, Quantification of tumor volume in nude mice bearing xenograft tumors of A375 cells that harbor a construct that allows doxycycline (DOX)-inducible expression of a STAG2-specific shRNA (shStag2 #60), after treatment with vehicle (−Dox), doxycycline (+Dox), vemurafenib (Vem), or both doxycycline and vemurafenib (+Dox+vem). (−Dox, n=5 mice: Vem, n=6 mice: +Dox, n=6mice; +Dox+Vem, n=7 mice). Data are mean±s.e.m. **P<0.01 by unpaired two-tailed Student's 1-test for comparison between the two groups of mice that were treated with vemurafenib. The data variance is similar between groups. FIG. 3B, Waterfall plots showing the percentage change in tumor volume at Day 7 for the individual tumors in each treatment group of the STAG2 knockdown experiment in FIG. 3A. FIG. 3C, Representative images of p-ERK (top) and STAG2 (bottom) expression as determined by immunohistochemical analysis in mouse tumor samples from the Stag2 knockdown experiment in FIG. 3A. Scale bars. 50 μm. FIG. 3D, Quantification of tumor volume in nude mice bearing xenograft tumors of A375 cells stably expressing either a scrambled control shRNA (shCtrl) or shSTAG3 #69 (shSTAG3) and that were fed a control or vemurafenib-containing (Vem) diet. n=5 mice in each group. Data are mean±s.e.m. The Student's t-test was performed to compare between two groups of mice that were treated with vemurafenib. The data variance is similar between groups. FIG. 3E, Waterfall plots showing the percentage change in tumor volume at Day 8 for the individual tumors in mice from each treatment group of the STAG3 knockdown experiment in FIG. 3D. FIG. 3F, Representative images of p-ERK (top) and STAG3 (bottom) expression, as determined by immunohistochemical analysis, in mouse tumor samples from the STAG3 knockdown experiment.

FIGS. 4A-4H. Loss of Stag2 does not affect Ras activity or expression of EGFR, MITF, or COT in melanoma cells. FIG. 4A, A375 and SKMEL28 cells were stably infected with lentivirus expression STAG2 shRNA #23 or scrambled control. RAS activation was assessed by pull-down assays with GST-RAF1-RAS binding domain (RBD), followed by Western blotting with indicated antibodies. Experiment was performed three times. FIG. 4B, A375 cells expressing Stag2 inducible shRNA pTRIPZ-shStag2 #60 were cultured in the presence or absence of doxycycline for 5 days, followed by treatment with 0.3 μM vemurafenib for 2 h. FIG. 4C, HEK293 cells were transfected with myc-tagged BRAF V600E together with FLAG-tagged wild-type Stag2 or D193N (DN) mutant. Cell lysates were immunoprecipitated with anti-myc antibodies, followed by western blotting. FIG. 4D, HEK293 cells were transfected with myc-tagged BRAF V600E together with FLAG-tagged wild-type Stag2 or D193N (DN) mutant. Cells were treated with 3 μM vemurafenib for 2 h. FIG. 4E, Braf-null MEFs stably expressing myc-BRAF V600E (VE) or V600E/R509H (VE/RH) were infected with lentivirus expressing Stag2 shRNA #09 or scramble control. Lysates were immunoprecipitated with anti-BRAF antibodies, followed by western blotting. FIG. 4F, Braf-null MEFs stably expressing various constructs were treated with 3 μM vemurafenib for 2 h. One representative from at least three independent experiments is shown. FIG. 4G, A375 or SKMEL28 cells expressing inducible STAG2 shRNA pTRIPZ-shSTAG2 #60 were cultured in the presence or absence of doxycycline for 5 d before lysates were used for Western blotting with indicated antibodies. Experiment was performed 3 times. FIG. 4H, A375 cells expressing STAG3 shRNA #96 or scrambled control were used for Western blotting with indicated antibodies. Experiment was performed 3 times.

FIGS. 5A-5B. Identification and characterization of Stag2 mutations in melanoma. FIG. 5A, HEK293 cells were transfected with FLAG-tagged wild-type Stag2 (WT) or Asp193Asn (DN) mutant. Cell lysates were immunoprecipitated with anti-FLAG antibodies, followed by Western blotting. Experiment was performed three times. FIG. 5B, A375. WM902 or M14 cells were incubated in the presence of 0.3 μM vemurafenib. Cell lysates were collected at indicated times and analyzed by Western blotting. Experiment was performed 3 times.

FIGS. 6A-6D. Identification and Characterization of Stag2 and Stag3 mutations in melanoma. FIG. 6A, Detection of a STAG2 mutation WM902BR cells by Sanger sequencing. FIG. 6A discloses SEQ ID NOs: 25 and 26, respectively, in order of appearance. FIG. 6B, Summary of immunohistochemical analyses of Stag2 and Stag3 in nine pairs of pre-treatment and post-relapse tumor samples from patients treated with BRAFi monotherapy or BRAFi/MEKi combination therapy. Each symbol represents one patient. FIG. 6C, HEK293 cells were transfected with FLAG-tagged wild-type STAG3 (WT). Pro272Ser (PS) or Arg508Gln (RQ) mutants. Cell lysates were immunoprecipitated with anti-FLAG antibodies, followed by Western blotting. Experiment was performed 3 times. FIG. 6D, Percentages of post-relapse samples from a total of nine patients treated with BRAFi monotherapy or BRAFi and MEKi combination therapy that showed changes of STAG2 or STAG3 expression, compared to their paired pre-treatment samples, in IHC analyses.

FIGS. 7A-7Q. Knockdown of Stag2 or Stag3 decreases BRAFi sensitivities in BRAF mutant melanoma cells. FIGS. 7A & 7B, Viability of SKMEL28 (FIG. 7A) or M14 (FIG. 7B) cells expressing after treatment with varying concentrations of dabrafenib for 3 d. Experiment was performed 3 times. Data are mean±s.e.m. FIGS. 7C & 7D, SKMEL28 (FIG. 7C) or M14 (FIG. 7D) cells expressing Stag2 inducible shRNA pTRIPZ-shStag2 #60 were treated with dabrafenib for 2 hr. Cell lysates were used for Western blotting with indicated antibodies. Experiment was performed three times. FIGS. 7E & 7F, Viability of A375 cells after treatment with varying concentrations of vemurafenib (FIG. 7E) or dabrafenib (FIG. 7F) for 3 days. FIGS. 7G & 7H, A375 cells expressing Stag2 inducible shRNA pTRIPZ-shStag2 #60 were treated with vemurafenib (FIG. 7G) or dabrafenib (FIG. 7H) for 2 hr. Cell lysates were used for Western blotting with indicated antibodies. Experiment was performed three times. FIG. 7I, Viability of M14 cells after treatment of varying concentrations of vemurafenib for 3 d. Experiment was performed 3 times. Data are mean±s.e.m. FIG. 7J, M14 cells expressing Stag3 shRNA #71 or scramble control were treated with vemurafenib for 2 hr. Cell lysates were used for Western blotting with indicated antibodies. Experiment was performed three times. FIGS. 7K & 7L, A375 (FIG. 7K) or SKMEL28 (FIG. 7L) cells expressing Stag3 shRNA #71 or scrambled control were treated with vemurafenib for 2 hr. Cell lysates were used for Western blotting with indicated antibodies. Experiment was performed three times. FIG. 7M, A375 cells expressing STAG3 inducible shRNA pTRIPZ-shSTAG3 #55 were infected with STAG2 shRNA #23 or scrambled control. Cells were cultured in the presence or absence of doxycycline for 5 d and treated with various concentrations of vemurafenib for 2 h before lysates were used for Western blotting with indicated antibodies. Experiment was performed 3 times. FIG. 7N, Viability of A375 cells after treatment with varying concentrations of trametinib for 3 d. Experiment was performed 3 times. Data are mean±s.e.m. FIG. 7O, A375 cells expressing STAG2 inducible shRNA pTRIPZ-shSTAG2 #60 were treated with trametinib for 2 h. Cell lysates were used for Western blotting with indicated antibodies. Experiment was performed 3 times. FIG. 7P, Viability of A375 cells after treatment of varying concentrations of dabrafenib and trametinib together at a ratio of 10:1 for 3 d. Experiment was performed 3 times. Data are mean±s.e.m. FIG. 7Q, A375 cells expressing STAG2 inducible shRNA pTRIPZ-shSTAG2 #60 were treated with dabrafenib and trametinib as indicated for 2 h. Cell lysates were used for Western blotting with indicated antibodies. Experiment was performed 3 times.

FIGS. 8A-8D. Loss of Stag2 or Stag3 activates MEK-ERK signaling through promoting BRAF-CRAF dimerization. FIG. 8A, Braf-null MEFs stably expressing myc-BRAF V600E and HA-KSR1 were infected with lentivirus expressing Stag2 shRNA #23 or scramble control and treated with 3 μM vemurafenib for 2 h. Cell lysates were immunoprecipitated with anti-BRAF antibodies, followed by western blotting. FIG. 8B, A375 cells expressing shStag3 #69 or scramble control were treated with 0.3 μM vemurafenib for 2 h. Cell lysates were immunoprecipitated with anti-BRAF antibodies, followed by western blotting. FIG. 8C, Braf-null MEFs expressing FLAG-BRAF wild-type (WT) or V600E (VE) were infected with lentivirus expressing Stag2 shRNA #09 or scramble control. Lysates were immunoprecipitated with anti-BRAF antibodies, followed by western blotting. FIG. 8D, LOX-IVMI cells stably expressing FLAG-tagged wild-type Stag2 or D193N mutant and treated with 1 1&M vemurafenib for 2 h. One representative from at least three independent experiments is shown.

FIGS. 9A-9B Regulation of PD-L1 protein levels by Stag2. FIG. 9A, Increased PD-L1 levels in BRAF inhibitor (BRAFi)-resistant melanoma cells with Stag2 mutations. Protein levels of PD-L1 were compared between WM902 parental cells (P) and WM902 BRAFi-resistant (BR) cells, and between WM983 parental cells (P) and WM983 BRAFi-resistant (BR) cells. WM902BR contains a K1083* loss-of-function mutation in STAG2. WM983BR cells have reduced Stag2 expression levels compared to WM983 cells. FIG. 9B, Knockdown of STAG2 by shRNA increases PD-L1 protein levels in A375 and M14 melanoma cells. Cell lysates were subjected to Western blotting with antibodies as indicated.

FIG. 10 Regulation of PD-L1 surface expression by Stag2. A375. M14 or SKMEL28 melanoma cells expressing pTRIPZ-shStag2 #60 were cultured in the presence or absence of doxycycline (Doxy) for 5 days to induce the expression of Stag2 shRNAs, followed by flow cytometry analysis for PD-L1 surface expression. Isotype antibody was used as a negative control. Knockdown of STAG2 increases PD-L1 surface levels in melanoma cells.

FIGS. 11A-11B Stag2 mutant WM902BR cells are sensitive to inhibition with SCH772984. Both WM902 (Stag2 WT) and WM902BR (Stag2 mutant) were treated with various concentrations of ERK inhibitor SCH772984 for 3 days, before MTS assays were performed. Data were from two independent experiments: Experiment A (FIG. 11A) and Experiment B (FIG. 11B).

FIGS. 12A-12B Knockdown of Stag2 in melanoma cells does not affect their sensitivities to the ERK inhibitor SCH772984. M14 (FIG. 12A) or SKMEL28 (FIG. 12B) melanoma cells expressing pTRIPZ-shStag2 #60 were cultured in the presence or absence of doxycycline (Doxy) for 5 days to induce the expression of Stag2 shRNAs and treated with various concentrations of ERK inhibitor SCH772984 for 3 days, before MTS assays were performed.

FIGS. 13A-13I Stag2 regulates ERK activity by controlling expression of DUSP6. FIG. 13A, Levels of DUSP4 and DUSP6, as assessed by qPCR analysis of total RNA that was isolated from A375 cells expressing scrambled shRNA or shRNAs specific for STAG2 or STAG3 (n=3 biological replicates). mRNA levels were calculated relative to those in cells expressing the scrambled control; levels of the housekeeping gene GAPDH were used as a reference. Data are mean±s.e.m. ****P<0.0001 by two-tailed Student's t-test. The data variance is similar between groups. FIG. 13B, Representative Western blot analysis of M14 or A375 cells expressing a doxycycline-inducible STAG2-specific shRNA that were cultured in the presence or absence of doxycycline for 5 d. GAPDH was used as a loading control. Experiment was performed three times. FIG. 13C, Representative Western blot analysis of M14 or A375 cells expressing shSTAG3 #71 (shSTAG3; +) or a scrambled control (−). Experiment was performed three times. FIG. 13D, Representative Western blot analysis of HEK293 cells that were transfected with indicated constructs. Experiment was performed three times. FIG. 13E, Genomic structure of the DUSP6 gene, showing the locations of regions amplified by ChIP-qPCR. R1, CTCF-binding region; R2, nonspecific region. FIG. 13F, ChIP-qPCR analysis, assessing the enrichment of the indicated DUSP6 regions with a CTCF-specific antibody relative to that with rabbit IgG, from A375 cells expressing a doxycycline-inducible STAG2-specific shRNA after 5 d of vehicle (−Dox) or doxycycline (+Dox) treatment. Chromatins were immunoprecipitated using CTCF antibody or rabbit IgG (n=3 biological replicates). Enrichment of H19 sequences after ChIP with anti-CTCF was used as a control. Results are expressed as fold enrichment relative to that of the nonspecific region (R2). Data are mean±s.e.m. *P<0.05 by two-tailed Student's t-test. The data variance is similar between groups. FIG. 13G, ChIP-qPCR analysis, as in FIG. 13F, from LOX-IVMI cells that stably expressed constructs encoding the indicated Flag-tagged STAG2 variants (n=3 biological replicates). Data are mean±s.e.m. **P<0.01 and ****P<0.0001 by two-tailed Student's t-test. The data variance is similar between groups. FIG. 13H, Representative Western blot analysis of A375 cells that stably express a doxycycline-inducible STAG2-specific shRNA, after infection with either a lentivirus encoding MYC-DUSP6 or a control vector. Cells were cultured in the presence or absence of doxycycline for 5 d and treated with 0.3 μM vemurafenib for 2 h before preparation of lysates. Experiment was performed three times. FIG. 13I, Representative images of clonogenic growth assays for inducible-shSTAG2-expressing A375 cells that also express a control vector (top) or MYC-tagged DUSP6 (bottom), after treatment with the indicated concentrations of vemurafenib. Experiment was performed three times. Scale bar. 5 mm.

FIGS. 14A-14F. Knockdown of STAG2 decreases MEKi sensitivities in NRAS mutant melanoma cells. FIG. 14A, SKMEL103 cells expressing STAG2 shRNA #23 or scrambled control were treated with trametinib for 2 h. Cell lysates were used for Western blotting with indicated antibodies. Experiment was performed 3 times. FIG. 14B, Viability of SKMEL103 cells after treatment of varying concentrations of trametinib for 3 d. Experiment was performed 3 times. Data are mean±s.e.m. FIG. 14C, 501MEL cells expressing STAG2 shRNA #23 or scrambled control were treated with trametinib for 2 h. Cell lysates were used for Western blotting with indicated antibodies. Experiment was performed 3 times. FIG. 14D, Viability of 501MEL cells after treatment of varying concentrations of trametinib for 3 d. Experiment was performed 3 times. Data are mean±s.e.m. FIG. 14E, Viability of 501MEL cells expressing STAG2 shRNA #23 or scrambled control were seeded at 3×10⁴ per well in 6-well plates and treated with trametinib as indicated in clonogenic growth assays. Experiment was performed 3 times. Scale bar: 5 mm. FIG. 14F, Conformation of NRAS mutation (SEQ ID NO: 27) in 501MEL cells by Sanger sequencing.

FIGS. 15A-15D Loss of STAG3 impairs the changes in cell cycle progression and reduced the percentages of annexin V-positive apoptotic cells in response to vemurafenib treatment. FIGS. 15A & 15B, A375 cells expressing STAG2 inducible shRNA pTRIPZ-shSTAG2 #60 were cultured in the presence or absence of doxycycline for 5 d. Cells were treated with or without 1 μM vemurafenib for 72 h before cell cycle (FIG. 15A) and apoptosis (FIG. 15B) analyses were performed. Experiment was performed 3 times. Data are mean±s.e.m. The P values were determined using two tailed Student's t-test. *P<0.05: **P<0.01; ****P<0.0001. The data variance is similar between groups. FIGS. 15C & 15D, A375 cells expressing STAG3 inducible shRNA pTRIPZ-shSTAG3 #55 were cultured in the presence or absence of doxycycline for 5 d. Cells were treated with or without 1 μM vemurafenib for 72 h before cell cycle (FIG. 15C) and apoptosis (FIG. 15D) analyses were performed. Experiment was performed 3 times. Data are mean±s.e.m. The P values were determined using two-tailed Student's t-test. *P<0.05: **P<0.01. The data variance is similar between groups.

FIGS. 16A-16E Ectopic expression of STAG2 or STAG3 increases sensitivities to BRAFi in melanoma cells. FIG. 16A, WM902-BR cells stably expressing FLAG-tagged wild-type STAG3 or control vector were treated with 3 μM vemurafenib for 2 h. Cell lysates were used for Western blotting with indicated antibodies. Experiment was performed 3 times. FIG. 16B, WM902-BR stable expressing of FLAG-tagged wild-type STAG3 or control vector were used in soft agar assays in the presence or absence of 3 μM vemurafenib. Experiment was performed 3 times. Scale bar: 5 mm FIG. 16C, WM983-BR cells stably expressing FLAG-tagged wild-type STAG2. STAG3 or vector control were treated with 1 μM vemurafenib for 2 h. Cell lysates were used for Western blotting with indicated antibodies. Experiment was performed 3 times. FIG. 16D, LOX-IVMI cells stably expressing FLAG-tagged wildtype STAG2 (WT). Lys1083* (K*) or Asp193Asn (DN) mutants were treated with 3 μM vemurafenib for 2 h. Cell lysates were used for western blotting with indicated antibodies. Experiment was performed 3 times. FIG. 16E, HEK293 cells were transfected with MYC-tagged BRAF Val600Glu together with FLAG-tagged wild-type STAG2 (WT). Lys1083* (K*) or Asp193Asn (DN) mutants. Cells were treated with 10 μM vemurafenib for 2 h. Cell lysates were used for Western blotting with indicated antibodies. Experiment was performed 3 times.

FIGS. 17A-17D, STAG2 regulates expression of DSP6 in melanoma cells. FIGS. 17A & 17B, Total RNA from A375 (FIG. 17A) and M14 (FIG. 17B) cells expressing STAG2 inducible shRNA pTRIPZ-shSTAG2 #60 were isolated, reverse transcribed, and expression levels of DUSP4 and DUSP6 were analyzed by qPCR. Levels of mRNA were calculated relative to the absence of doxycycline control, and housekeeping GAPDH gene was used as the reference. n=3 biological replicates. Data are mean±s.e.m. The P values were determined using two-tailed Student's 1-test. **P 0.01: ****P 0.0001. The data variance is similar between groups. FIG. 17C, Lysates from SKMEL103 or 501MEL cells expressing STAG2 shRNA #23 or scrambled control were used for Western blotting with indicated antibodies. Experiment was performed 3 times. FIG. 17D, Expression of DUSP6 protein in BRAFi-resistant cell lines (BR) and their parental BRAFi-sensitive counterparts (P). Lysates were used for Western blotting with indicated antibodies. Experiment was performed 3 times.

FIGS. 18A-18B, STAG2 regulates the binding of CTCF to the DUSP6 locus. FIG. 18A, M14 cells expressing inducible STAG2 shRNA pTRIPZ-shSTAG2 #60 were cultured in the presence or absence of doxycycline for 5 d before ChIP-qPCR assays were performed. Chromatins were immunoprecipitated using CTCF antibody or rabbit IgG. FIG. 18B, Chromatins of WM902 and WM902-BR were immunoprecipitated using CTCF antibody or rabbit IgG. IP-ed chromatins were examined using qPCR with primers for R1 and R2 regions of DUSP6 and H19. Results are expressed as fold enrichment relative to the non-specific region (R2). n 3 biological replicates. Data are mean±s.e.m. The P values were determined using two-tailed Student's t-test. *P 0.05: **P 0.01. The data variance is similar between groups.

FIGS. 19A-19E STAG2 regulates RK activity through controlling expression of DUSP6. FIG. 19A, M14 cells expressing STAG2 inducible shRNA pTRIPZshSTAG2 #60 were infected with lentivirus expressing MYC-DUSP6 or control vector. Cells were cultured in the presence or absence of doxycycline for 5 d and treated with 0.3 μM vemurafenib for 2 h before lysates were used for Western blotting with indicated antibodies. Experiment was performed 3 times. FIGS. 19B & 19C, WM902-BR cells (FIG. 19B) and WM983-BR (FIG. 19C) expressing MYC-tagged DUSP6 or vector control were treated with 10 μM vemurafenib for 2 h. Cell lysates were used for western blotting with indicated antibodies. Experiment was performed 3 times. FIG. 19D, WM902-BR cells expressing MYC-tagged DUSP6 or vector control were seeded at 3×10⁴ per well in 6-well plates and treated with in the presence or absence of 1 μM vemurafenib as indicated in clonogenic growth assays. Experiment was performed 3 times. Scale bar: 5 mm FIG. 19E, A375 cells expressing STAG2 shRNA #23 or scrambled control were infected with lentivirus expressing Flag-DUSP4 or control vector. Cells were treated with vemurafenib for 2 h before lysates were used for Western blotting with indicated antibodies. Experiment was performed 3 times.

FIG. 20 Schematic model for regulation of BRAF-MEK0ERK signaling pathway by STAG2.

### DETAILED DESCRIPTION

Provided herein are methods for treating cancer and for selecting a cancer treatment based on the expression of Stag2/3 proteins and/or the presence of mutations in the genes encoding the Stag2/3 proteins. In particular, these methods relate to cancer treatments using BRAF kinase inhibitors and to methods of predicting whether a subject will respond to anti-cancer treatment comprising a BRAF inhibitor, or if an anti-cancer treatment with a different mechanism of action (e.g., PD-1 inhibitor, PD-L1 inhibitor, or ERK inhibitor) should be administered instead.

### Definitions

The term “biological sample” as used herein refers to a cell or population of cells or a quantity of tissue or fluid from a subject. Most often, the sample has been removed from a subject, but the term “biological sample” can also refer to cells or tissue analyzed in vivo, i.e., without removal from the subject. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g., buccal scrapes), whole blood, plasma, serum, urine, saliva, cell culture, or cerebrospinal fluid. A biological sample or tissue sample can refer to a sample of tissue or fluid isolated from an individual including, but not limited to, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells (including, but not limited to, blood cells), tumors, organs, and also samples of in vitro cell culture constituent. In some embodiments, the sample is from a resection, bronchoscopic biopsy, or core needle biopsy of a primary or metastatic tumor. In addition, fine needle aspirate samples can be used. Samples can be either paraffin-embedded or frozen tissue. The term “sample” includes any material derived by processing such a sample. Derived samples may, for example, include nucleic acids or proteins extracted from the sample or obtained by subjecting the sample to techniques such as amplification or reverse transcription of mRNA, isolation and/or purification of certain components, etc.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, a subject having a cancer “known to be responsive to a BRAF inhibitor” refers to a subject whose cancer or tumor is responding to treatment with a therapeutically effective amount of a BRAF inhibitor as determined by a reduction in at least one symptom of the cancer, for example, a reduction in tumor or lesion size, inhibition of growth of the tumor or lesion, etc. In some embodiments, the subject having a cancer “known to be responsive to a BRAF inhibitor” does not have decreased expression and/or activity of Stag2 and/or Stag3 and/or does not have an inherent resistance to BRAF inhibitors.

As used herein, a subject having a cancer “in a phase of responsiveness to a BRAF inhibitor” refers to a subject whose tumor or cancer is currently responding to treatment with a therapeutically effective amount of a BRAF inhibitor as determined by a reduction in at least one symptom of the cancer, for example, a reduction in tumor or lesion size, inhibition of growth of the tumor or lesion, etc. However, the subject having a cancer “in a phase of responsiveness” can move into a phase of non-responsiveness to the BRAF inhibitor. In some embodiments, the subject having a cancer in a phase of responsiveness is a subject that has one or more mutations in Stag2 or Stag3. In other embodiments, a subject having a cancer in a phase of responsiveness is continually monitored for a decrease in Stag2 and/or Stag3 expression, which can indicate a shift of the cancer into a phase of non-responsiveness and in turn indicates the development of resistance to a BRAF inhibitor (e.g., acquired resistance).

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

As used herein, the term “specifically binds” refers to the ability of an antibody or antibody fragment thereof to bind to a target, e.g., Stag2 and/or Stag3, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. The person of ordinary skill in the art can determine appropriate conditions under which the polypeptide agents directed to Stag2 and/or Stag3 selectively bind their targets, e.g., using any suitable methods, such as titration of an antibody in a suitable cell binding assay.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

### Cancers

Some non-limiting examples of cancer that can be treated using the methods and compositions described herein include, but are not limited to, carcinoma, lymphoma, blastoma, sarcoma, and leukemia. Other exemplary cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments, the carcinoma or sarcoma includes, but is not limited to, carcinomas and sarcomas found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus. The types of carcinomas include, but are not limited to, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. The types of sarcomas include, but are not limited to, soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma.

In one embodiment of the methods, the subject having the tumor, cancer or malignant condition is undergoing, or has undergone, treatment with a conventional cancer therapy. In some embodiments, the cancer therapy is chemotherapy, radiation therapy, immunotherapy or a combination thereof.

Inhibitors of BRAF, PD-1, PD-L1, MEK and/or ERK can be used alone or in combination with other therapies, including chemotherapy, radiation, cancer immunotherapy, or combinations thereof. Such therapies can either directly target a tumor (e.g., by inhibition of a tumor cell protein or killing of highly mitotic cells) or act indirectly, e.g., to provoke or accentuate an anti-tumor immune response.

Exemplary anti-cancer agents that can be used in combination with a BRAF, PD-1, PD-L1, MEK and/or ERK inhibitor include alkylating agents such as thiotepa and CYTOXAN™; cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8): dolastatin: duocarmycin (including the synthetic analogues, KW-2189 and CB1-TM1): eleutherobin: pancratistatin: a sarcodictyin: spongistatin: nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard: nitrosoureas, such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitracrine; pentostatin; phenamet; pirarubicin; losoxantrone; podophyllinic acid; 2-ethylhydrazide; procarbazine; PSK; polysaccharide complex (JHS Natural Products™, Eugene. Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids. e.g., TAXOL™ paclitaxel (Bristol-Meyers Squibb Oncology. Princeton. N.J.). ABRAXANE™ Cremophor-free, albumin-engineered nanoparticle formulation of paclitaxel (American Pharmaceutical Partners, Schaumberg. Ill.), and TAXOTERE™ doxetaxel (Rhone-Poulenc Rorer, Antony, France); chloranbucil; GEMZAR™, gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™, vinorelbine; novantrone; teniposide; edatrexate; daunomycin; aminopterin; xeloda; ibandronate; irinotecan (CAMPTOSAR™, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX™); lapatinib (TYKERB™); inhibitors of PKC-alpha, Raf, H-Ras, EGFR (e.g., erlotinib (TARCEVA™)) and VEGF-A that reduce cell proliferation, and pharmaceutically acceptable salts, acids or derivatives of any of the above. In addition, the methods of treatment can further include the use of radiation.

Detecting a Mutation in Stag2 and/or Stag3

Essentially any method known in the art for determining genetic polymorphism can be used for detecting a mutation in Stag2 and/or Stag3. Examples include, but are not limited to, PCR or other amplification methods, hybridization methods using an allele-specific oligonucleotide matrix (e.g., TAQMAN™ PCR method, INVADER™ assay method), primer extension reaction methods, sequencing methods, MALDI-TOF/MS methods and DNA chip methods (e.g., microarrays). Non-limiting examples of detection methods that are applicable to analysis of such mutations are discussed herein. In one embodiment, the detection of a mutation or determination of reduced expression (see section “Measuring Expression of Stag2 and/or Stag3, below) requires physically contacting a sample with one or more reagents necessary to indicate the expression of polymorphic status of the sample. This excludes, for example, looking up the expression status in a database or other record.

Direct Sequencing Assays: In some embodiments, variant sequences are detected using a direct sequencing technique. In these assays, DNA samples are first isolated from a subject using any suitable method. In some embodiments, the region of interest is cloned into a suitable vector and amplified by growth in a host cell (e.g., bacteria). In other embodiments, DNA in the region of interest is amplified using PCR.

Following amplification. DNA in the region of interest (e.g., the region containing the mutation of interest) is sequenced using any suitable method, including, but not limited to, manual sequencing using radioactive marker nucleotides, or automated sequencing. The results of the sequencing are displayed using any suitable method. The sequence is examined and the presence or absence of a given mutation is determined.

In one embodiment, the DNA is amplified, for example, by PCR using one or more region-specific primers. In one embodiment, the binding of the PCR primer(s) to the isolated and and/or subcloned DNA induces a conformation change in the DNA sequence/template that is indicative of hybridization. In one embodiment, the DNA is amplified using the following pairs of primers:
(i) 5′-GATAGTGGAGATTATCCACTTAC-3′ (also referred to herein as primer 7F; SEQ ID NO. 5) and 5′-CTGCCAGGGTGCTTGTATGTCG-3′ (also referred to herein as primer 7R; SEQ ID NO. 6), or
(ii) 5′-ATGCCTATGCTCGCACAACT-3′ (also referred to herein as primer 29F; SEQ ID NO. 7) and 5-ATACTGAGTCCATTTCCCTATGC-3′ (also referred to herein as primer 29R; SEQ ID NO. 8), wherein the amplification comprises a step of binding the primer pair to the DNA template, which induces a conformational change in the DNA template that is indicative of hybridization.

PCR Assay: Variant sequences can also be detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

In some embodiments, the binding of the PCR primer set induces a conformational change in the DNA sequence/template that is indicative of hybridization. In one embodiment, the DNA is amplified using the following pairs of primers:
(i) 5′-GATAGTGGAGATTATCCACTTAC-3′ (also referred to herein as primer 7F; SEQ ID NO. 5) and 5′-CTGCCAGGGTGCTTGTATGTCG-3′ (also referred to herein as primer 7R; SEQ ID NO. 6), or
(ii) 5′-ATGCCTATGCTCGCACAACT-3′ (also referred to herein as primer 29F; SEQ ID NO. 7) and 5′-ATACTGAGTCCATTTCCCTATGC-3′ (also referred to herein as primer 29R; SEQ ID NO. 8), wherein the amplification comprises a step of binding the primer pair to the DNA template, which induces a conformational change in the DNA template that is indicative of hybridization.

Fragment Length Polymorphism Assays: In some embodiments, variant sequences are detected using a fragment length polymorphism assay. In a fragment length polymorphism assay, a unique DNA banding pattern based on cleaving the DNA at a series of positions is generated using an enzyme (e.g., a restriction enzyme or a CLEAVASE I™ enzyme (Third Wave Technologies, Madison, Wis.). DNA fragments from a sample containing a mutation will have a different banding pattern than wild type.

(a) RFLP Assay: In some embodiments, variant sequences are detected using a restriction fragment length polymorphism assay (RFLP). The region of interest is first isolated using PCR. The PCR products are then cleaved with restriction enzymes known to give a unique length fragment for a given polymorphism. The restriction-enzyme digested PCR products are generally separated by gel electrophoresis and may be visualized by ethidium bromide staining. The length of the fragments is compared to molecular weight markers and fragments generated from wild-type and mutant controls. In one embodiment, the DNA is amplified via PCR using the following pairs of primers:
(i) 5′-GATAGTGGAGATTATCCACTTAC-3′ (also referred to herein as primer 7F; SEQ ID NO. 5) and 5′-CTGCCAGGGTGCTTGTATGTCG-3′ (also referred to herein as primer 7R; SEQ ID NO. 6), or
(ii) 5′-ATGCCTATGCTCGCACAACT-3′ (also referred to herein as primer 29F; SEQ ID NO. 7) and 5′-ATACTGAGTCCATTTCCCTATGC-3′ (also referred to herein as primer 29R; SEQ ID NO. 8), wherein the amplification comprises a step of binding the primer pair to the DNA template, which induces a conformational change in the DNA template that is indicative of hybridization.

(b) CFLP Assay: In other embodiments, variant sequences are detected using a CLEAVASE™ fragment length polymorphism assay (CFLP; Third Wave Technologies, Madison, Wis.; See e.g., U.S. Pat. Nos. 5,843,654:5,843,669; 5,719,208; and 5,888,780; each of which is herein incorporated by reference). This assay is based on the observation that when single strands of DNA fold on themselves, they assume higher order structures that are highly individual to the precise sequence of the DNA molecule. These secondary structures involve partially duplexed regions of DNA such that single stranded regions are juxtaposed with double stranded DNA hairpins. The CLEAVASE I™ enzyme, is a structure-specific, thermostable nuclease that recognizes and cleaves the junctions between these single-stranded and double-stranded regions. In one embodiment, contacting the DNA template with a CLEAVASE™ endonuclease enzyme causes a conformational change in the DNA sequence by breaking the junctions between single-stranded and double-stranded regions in the DNA, thereby changing the secondary structure of the DNA.

The region of interest is first isolated, for example, using PCR. In one embodiment, the DNA is amplified using the following pairs of primers:
(i) 5′-GATAGTGGAGATTATCCACTTAC-3′ (also referred to herein as primer 7F; SEQ ID NO. 5) and 5′-CTGCCAGGGTGCTTGTATGTCG-3′ (also referred to herein as primer 7R; SEQ ID NO. 6), or

(ii) 5′-ATGCCTATGCTCGCACAACT-3′ (also referred to herein as primer 29F; SEQ ID NO. 7) and 5-ATACTGAGTCCATTTCCCTATGC-3′ (also referred to herein as primer 29R; SEQ ID NO. 8), wherein the amplification comprises a step of binding the primer pair to the DNA template, which induces a conformational change in the DNA template that is indicative of hybridization. In some embodiments, one or both strands are labeled. Then, DNA strands are separated by heating. Next, the reactions are cooled to allow intrastrand secondary structure to form. The PCR products are then treated with the CLEAVASE™ I enzyme to generate a series of fragments that are unique to a given mutation. The CLEAVASE™ enzyme treated PCR products are separated and detected (e.g., by denaturing gel electrophoresis) and visualized (e.g., by autoradiography, fluorescence imaging or staining). The length of the fragments is compared to molecular weight markers and fragments generated from wild-type and mutant controls.

Hybridization Assays: In certain embodiments, variant sequences are detected using a hybridization assay. In a hybridization assay, the presence of absence of a given mutation is determined based on the ability of the DNA from the sample to hybridize to a complementary DNA molecule (e.g., an oligonucleotide probe). In one embodiment, contacting a DNA template with a complementary DNA molecule, such as an oligonucleotide probe, causes a conformation change in the DNA template. Such conformational changes introduce non-natural changes to the DNA template secondary structure such that the DNA template is not the same as that isolated from a subject. A selection of hybridization assays are provided below:

(a) Direct Detection of Hybridization: In some embodiments, hybridization of a probe to the sequence of interest (e.g., a mutation) is detected directly by visualizing a bound probe (e.g., a Northern or Southern assay; See e.g., Ausabel et al. (eds.). Current Protocols in Molecular Biology. John Wiley & Sons. NY (1991)). In these assays, genomic DNA (Southern) or RNA (Northern) is isolated from a subject. The DNA or RNA is then cleaved with a series of restriction enzymes that cleave infrequently in the genome and not near any of the markers being assayed. The DNA or RNA is then separated (e.g., on an agarose gel) and transferred to a membrane. A labeled (e.g., by incorporating a radionucleotide) probe or probes specific for the mutation being detected is allowed to contact the membrane under a condition or low, medium, or high stringency conditions. Unbound probe is removed and the presence of binding is detected by visualizing the labeled probe. In one embodiment, binding of the labeled probe to the DNA or RNA sequence induces a conformational change in the DNA or RNA sequence.

(b) Detection of Hybridization Using “DNA Chip” Assays: In some embodiments, variant sequences are detected using a DNA chip hybridization assay. In this assay, a series of oligonucleotide probes are affixed to a solid support. The oligonucleotide probes are designed to be unique to a given mutation. The DNA sample of interest is contacted with the DNA “chip” and hybridization is detected. In one embodiment, contacting an isolated DNA template with a DNA chip causes a conformation change in the DNA template. Such conformational changes introduce non-natural changes to the DNA template secondary structure such that the DNA template is not the same as that isolated from a subject or that occurs naturally in a subject.

In some embodiments, the DNA chip assay is a GENECHIP™ (Affymetrix. Santa Clara. Calif.; See e.g., U.S. Pat. Nos. 6,045,996; 5,925,525; and 5,858,659; each of which is herein incorporated by reference) assay. The GENECHIP™ technology uses miniaturized, high-density arrays of oligonucleotide probes affixed to a “chip.” Probe arrays are manufactured by Affymetrix's light-directed chemical synthesis process, which combines solid-phase chemical synthesis with photolithographic fabrication techniques employed in the semiconductor industry. Using a series of photolithographic masks to define chip exposure sites, followed by specific chemical synthesis steps, the process constructs high-density arrays of oligonucleotides, with each probe in a predefined position in the array. Multiple probe arrays are synthesized simultaneously on a large glass wafer. The wafers are then diced, and individual probe arrays are packaged in injection-molded plastic cartridges, which protect them from the environment and serve as chambers for hybridization.

The nucleic acid to be analyzed is isolated, amplified by PCR, and labeled with a fluorescent reporter group. In one embodiment, the DNA is amplified using the following pairs of primers:
(i) 5′-GATAGTGGAGATTATCCACTTAC-3′ (also referred to herein as primer 7F; SEQ ID NO. 5) and 5′-CTGCCAGGGTGCTTGTATGTCG-3′ (also referred to herein as primer 7R; SEQ ID NO. 6), or
(ii) 5′-ATGCCTATGCTCGCACAACT-3′ (also referred to herein as primer 29F; SEQ ID NO. 7) and 5′-ATACTGAGTCCATTTCCCTATGC-3′ (also referred to herein as primer 29R; SEQ ID NO. 8), wherein the amplification comprises a step of binding the primer pair to the DNA template, which induces a conformational change in the DNA template that is indicative of hybridization. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, CA) is utilized. Through the use of microelectronics. Nanogen's technology enables the active movement and concentration of charged molecules to and from designated test sites on its semiconductor microchip. DNA capture probes unique to a given mutation are electronically placed at, or “addressed” to, specific sites on the microchip. Since DNA has a strong negative charge, it can be electronically moved to an area of positive charge.

First, a test site or a row of test sites on the microchip is electronically activated with a positive charge. Next, a solution containing the DNA probes is introduced onto the microchip. The negatively charged probes rapidly move to the positively charged sites, where they concentrate and are chemically bound to a site on the microchip. The microchip is then washed and another solution of distinct DNA probes is added until the array of specifically bound DNA probes is complete.

A test sample is then analyzed for the presence of target DNA molecules by determining which of the DNA capture probes hybridize, with complementary DNA in the test sample (e.g., a PCR amplified gene of interest). An electronic charge is also used to move and concentrate target molecules to one or more test sites on the microchip. The electronic concentration of sample DNA at each test site promotes rapid hybridization of sample DNA with complementary capture probes (hybridization may occur in minutes). To remove any unbound or nonspecifically bound DNA from each site, the polarity or charge of the site is reversed to negative, thereby forcing any unbound or nonspecifically bound DNA back into solution away from the capture probes. A laser-based fluorescence scanner is used to detect binding. In still further embodiments, an array technology based upon the segregation of fluids on a flat surface (chip) by differences in surface tension (Protogene, Palo Alto, Calif.) is utilized (See e.g., U.S. Pat. Nos. 6,001,311; 5,985,551; and 5,474,796; each of which is herein incorporated by reference).

Protogene's technology is based on the fact that fluids can be segregated on a flat surface by differences in surface tension that have been imparted by chemical coatings. Once so segregated, oligonucleotide probes are synthesized directly on the chip by ink-jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for the mutation of interest are affixed to the chip using Protogene's technology. The chip is then contacted with the PCR-amplified genes of interest. Following hybridization, unbound DNA is removed and hybridization is detected using any suitable method (e.g., by fluorescence de-quenching of an incorporated fluorescent group).

In yet other embodiments, a “bead array” is used for the detection of polymorphisms (Illumina, San Diego, Calif.). Illumina uses a bead array technology that combines fiber optic bundles and beads that self-assemble into an array. Each fiber optic bundle contains thousands to millions of individual fibers depending on the diameter of the bundle. The beads are coated with an oligonucleotide specific for the detection of a given mutation. Batches of beads are combined to form a pool specific to the array. To perform an assay, the bead array is contacted with a prepared subject sample (e.g., DNA). Hybridization is detected using any suitable method.

Enzymatic Detection of Hybridization: In some embodiments of the present invention, hybridization is detected by enzymatic cleavage of specific structures (INVADER™ assay, Third Wave Technologies; See e.g., U.S. Pat. Nos. 5,846,717, 6,090,543; 6,001,567; 5,985,557; and 5,994,069; each of which is herein incorporated by reference). Contacting an isolated DNA or RNA sequence with an enzyme to induce cleavage of specific structures induces a conformational change in the secondary structure of the DNA or RNA sequence, thereby altering an isolated DNA or RNA sequence that exists endogenously to a conformationally different sequence. The INVADER™ flap endonuclease assay detects specific DNA and RNA sequences by using structure-specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′-end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System. Applied Biosystems. Foster City. Calif.).

The INVADER™ flap endonuclease assay detects specific mutations in unamplified genomic DNA. In an embodiment of the INVADER™ flap endonuclease assay used for detecting SNPs in genomic DNA, two oligonucleotides (a primary probe specific either for a SNP/mutation or wild type sequence, and an INVADER™ oligonucleotide) hybridize in tandem to the genomic DNA to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls.

In some embodiments, hybridization of a bound probe is detected using a TAQMAN™ assay (PE Biosystems. Foster City, Calif.; See e.g., U.S. Pat. Nos. 5,962,233 and 5,538,848, each of which is herein incorporated by reference). The assay is performed during a PCR reaction. The TAQMAN™ gene expression assay exploits the 5′-3′ exonuclease activity of DNA polymerases such as AMPLITAQ™ DNA polymerase. A probe, specific for a given allele or mutation, is included in the PCR reaction. The probe consists of an oligonucleotide with a 5′-reporter dye (e.g., a fluorescent dye) and a 3′-quencher dye. During PCR, if the probe is bound to its target, the 5′-3′ nucleolytic activity of the AMPLITAQ polymerase cleaves the probe between the reporter and the quencher dye. The separation of the reporter dye from the quencher dye results in an increase of fluorescence. The signal accumulates with each cycle of PCR and can be monitored with a fluorimeter.

In still further embodiments, polymorphisms are detected using the SNP-IT™ primer extension assay (Orchid Biosciences, Princeton, N.J.; See e.g., U.S. Pat. Nos. 5,952,174 and 5,919,626, each of which is herein incorporated by reference). In this assay, SNPs are identified by using a specially synthesized DNA primer and a DNA polymerase to selectively extend the DNA chain by one base at the suspected SNP location. DNA in the region of interest is amplified and denatured. Polymerase reactions are then performed using miniaturized systems called microfluidics. Detection is accomplished by adding a label to the nucleotide suspected of being at the SNP or mutation location. Incorporation of the label into the DNA can be detected by any suitable method (e.g., if the nucleotide contains a biotin label, detection is via a fluorescently labeled antibody specific for biotin).

Other Detection Assays: Additional detection assays that are produced and utilized using the systems and methods described herein include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); mass spectrometry assays (e.g., MASSARRAY™ system (Sequenom, San Diego, Calif.)) rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci USA 88, 189-93 (1991)); and sandwich hybridization methods (e.g., U.S. Pat. No. 5,288,609, herein incorporated by reference in its entirety).

Probes for detecting a mutation in Stag2 and/or Stag3: In some embodiments, a DNA sample is contacted with an oligonucleotide probe or oligonucleotide primer created so the 5′ terminus, 3′ terminus or central base contains the genetic polymorphism site. In certain embodiments, the oligonucleotide probe or oligonucleotide primer is created such that it selectively binds to one of the mutations (e.g., loss-of-function mutations) of Stag2 and/or Stag3 as listed in Table 1 and Table 2.

In some embodiments, a DNA sample is contacted with an oligonucleotide that flanks or is adjacent to a polymorphic site (e.g., those listed in Table 1 or Table 2), such that the presence of the polymorphism can be detected by modification of the oligonucleotide in a manner dependent on the presence or absence of the polymorphism. Also contemplated herein are kits comprising, at a minimum, at least one primer (e.g., at least 2, 3, 4, 5, 6, 7, 8, 9, 10, 15, 20 or more primers) for detecting one or more polymorphic sites as described herein.

TABLE 1
Mutations of Stag2 and/or Stag3 known
to be loss-of-function mutations

| Gene | Mutation |
|---|---|
| Stag2 | D193N |
| Stag2 | K1083* |

TABLE 2
Mutations in Stag2 and/or Stag3

| Gene | Mutation |
|---|---|
| STAG2 | E6D |
| STAG2 | G46Afs*25 |
| STAG2 | K48Gfs*33 |
| STAG2 | G54C |
| STAG2 | G61E |
| STAG2 | P63H |
| STAG2 | P67L |
| STAG2 | R69Q |
| STAG2 | H73Y |
| STAG2 | K89* |
| STAG2 | K92N |
| STAG2 | D101Lfs*8 |
| STAG2 | W102* |
| STAG2 | S105L |
| STAG2 | H108Afs*2 |
| STAG2 | R110* |
| STAG2 | F121Lfs*24 |
| STAG2 | Q123* |
| STAG2 | X129_splice |
| STAG2 | E134D |
| STAG2 | R137I |
| STAG2 | R137_Q140delinsK |
| STAG2 | S142* |
| STAG2 | R146* |
| STAG2 | M148Nfs*3 |
| STAG2 | M148Dfs*3 |
| STAG2 | M148Sfs*3 |
| STAG2 | M148Dfs*3 |
| STAG2 | M148I |
| STAG2 | T149Kfs*33 |
| STAG2 | T149Mfs*34 |
| STAG2 | D153N |
| STAG2 | X154_splice |
| STAG2 | S156G |
| STAG2 | P160L |
| STAG2 | L161P |
| STAG2 | Q167* |
| STAG2 | K169* |
| STAG2 | C176Vfs*7 |
| STAG2 | V181L |
| STAG2 | V181A |
| STAG2 | L182Yfs*8 |
| STAG2 | R184W |
| STAG2 | Q185H |
| STAG2 | D193N |
| STAG2 | E194D |
| STAG2 | M197Wfs*28 |
| STAG2 | I201M |
| STAG2 | D209Vfs*29 |
| STAG2 | Q211E |
| STAG2 | R216* |
| STAG2 | R216Q |
| STAG2 | H217R |
| STAG2 | S219Kfs*20 |
| STAG2 | L221M |
| STAG2 | M224I |
| STAG2 | L235Q |
| STAG2 | L237I |
| STAG2 | E251* |
| STAG2 | E251A |
| STAG2 | R252Q |
| STAG2 | R259* |
| STAG2 | E262* |
| STAG2 | X274_splice |
| STAG2 | E276D |
| STAG2 | Q278E |
| STAG2 | R298C |
| STAG2 | A300V |
| STAG2 | E303Mfs*16 |
| STAG2 | E303K |
| STAG2 | R305L |
| STAG2 | C308R |
| STAG2 | C308Rfs*10 |
| STAG2 | I312L |
| STAG2 | N325S |
| STAG2 | N325Tfs*4 |
| STAG2 | S327Vfs*3 |
| STAG2 | Y328* |
| STAG2 | K330Ifs*8 |
| STAG2 | V332Sfs*7 |
| STAG2 | X340_splice |
| STAG2 | Q352* |
| STAG2 | K358E |
| STAG2 | E365Nfs*14 |
| STAG2 | R370W |
| STAG2 | R370Q |
| STAG2 | R370G |
| STAG2 | E383* |
| STAG2 | D385Y |
| STAG2 | V386Cfs*15 |
| STAG2 | L405I |
| STAG2 | L405Sfs*20 |
| STAG2 | E408D |
| STAG2 | N412H |
| STAG2 | Y414F |
| STAG2 | S419* |
| STAG2 | R422Q |
| STAG2 | E430Dfs*18 |
| STAG2 | L432V |
| STAG2 | R439H |
| STAG2 | R440G |
| STAG2 | P442Qfs*6 |
| STAG2 | P456A |
| STAG2 | N457Kfs*13 |
| STAG2 | V465F |
| STAG2 | F467L |
| STAG2 | F468Lfs*5 |
| STAG2 | L469* |
| STAG2 | X472_splice |
| STAG2 | M484I |
| STAG2 | D486N |
| STAG2 | N499_L501dup |
| STAG2 | D515* |
| STAG2 | A532V |
| STAG2 | G540* |
| STAG2 | R541K |
| STAG2 | G544E |
| STAG2 | V547Gfs*13 |
| STAG2 | X547_splice |
| STAG2 | T549Yfs*11 |
| STAG2 | R560W |
| STAG2 | A568Rfs*19 |
| STAG2 | L571P |
| STAG2 | P572S |
| STAG2 | Q573R |
| STAG2 | Y578F |
| STAG2 | T586S |
| STAG2 | L588* |
| STAG2 | Q590* |
| STAG2 | Q593* |
| STAG2 | R604* |
| STAG2 | E606A |
| STAG2 | D610G |
| STAG2 | R614* |
| STAG2 | R614Q |
| STAG2 | V620L |
| STAG2 | T635I |
| STAG2 | Y636* |
| STAG2 | Y636Lfs*6 |
| STAG2 | E642K |
| STAG2 | S653* |
| STAG2 | R654I |
| STAG2 | K664Nfs*28 |
| STAG2 | F665Lfs*27 |
| STAG2 | R667W |
| STAG2 | E675D |
| STAG2 | H698R |
| STAG2 | X699_splice |
| STAG2 | S704L |
| STAG2 | W706L |
| STAG2 | W706G |
| STAG2 | L716V |
| STAG2 | E721* |
| STAG2 | E721V |
| STAG2 | X727_splice |
| STAG2 | E727D |
| STAG2 | Q728* |
| STAG2 | V730Tfs*14 |
| STAG2 | V730Ffs*10 |
| STAG2 | V740Efs*9 |
| STAG2 | L742I |
| STAG2 | X755_splice |
| STAG2 | X756_splice |
| STAG2 | K762Rfs*4 |
| STAG2 | Q770* |
| STAG2 | N778K |
| STAG2 | T782I |
| STAG2 | V783F |
| STAG2 | X786_splice |
| STAG2 | Q801* |
| STAG2 | M803Vfs*5 |
| STAG2 | M809I |
| STAG2 | Y815* |
| STAG2 | I829N |
| STAG2 | D831N |
| STAG2 | H832R |
| STAG2 | I855T |
| STAG2 | L858P |
| STAG2 | R861K |
| STAG2 | R862I |
| STAG2 | L865R |
| STAG2 | A866Cfs*5 |
| STAG2 | A866E |
| STAG2 | C869R |
| STAG2 | K887E |
| STAG2 | X891_splice |
| STAG2 | K901E |
| STAG2 | I910R |
| STAG2 | I910Tfs*20 |
| STAG2 | K917* |
| STAG2 | G935Rfs*3 |
| STAG2 | T944Ifs*4 |
| STAG2 | G947A |
| STAG2 | K949N |
| STAG2 | E950* |
| STAG2 | R953* |
| STAG2 | R954H |
| STAG2 | L961R |
| STAG2 | T966K |
| STAG2 | A971V |
| STAG2 | P987Q |
| STAG2 | P987T |
| STAG2 | Q988* |
| STAG2 | L995* |
| STAG2 | L997F |
| STAG2 | A998T |
| STAG2 | A998V |
| STAG2 | R1012* |
| STAG2 | R1016K |
| STAG2 | E1023* |
| STAG2 | T1027A |
| STAG2 | Q1029* |
| STAG2 | R1033* |
| STAG2 | R1034* |
| STAG2 | R1045* |
| STAG2 | R1045Q |
| STAG2 | D1055N |
| STAG2 | S1058* |
| STAG2 | R1071Q |
| STAG2 | T1079A |
| STAG2 | K1083* |
| STAG2 | E1086D |
| STAG2 | Q1089* |
| STAG2 | X1093_splice |
| STAG2 | E1107* |
| STAG2 | M1126I |
| STAG2 | E1128D |
| STAG2 | P1129S |
| STAG2 | H1149D |
| STAG2 | X1156_splice |
| STAG2 | R1195C |
| STAG2 | S1215L |
| STAG2 | E1224_D1229del |
| STAG2 | D1234N |
| STAG2 | X1235_splice |
| STAG2 | F1251L |
| STAG2 | A1255T |
| STAG2 | S1266* |
| STAG2 | M1267K |
| STAG3 | P4Rfs*42 |
| STAG3 | P4T |
| STAG3 | K13N |
| STAG3 | D30N |
| STAG3 | S33P |
| STAG3 | S55R |
| STAG3 | R58Afs*3 |
| STAG3 | R67* |
| STAG3 | P69Q |
| STAG3 | P73T |
| STAG3 | P73L |
| STAG3 | V74M |
| STAG3 | K79N |
| STAG3 | K80R |
| STAG3 | R83I |
| STAG3 | R90Q |
| STAG3 | E94Q |
| STAG3 | A107fs |
| STAG3 | A107T |
| STAG3 | Q112K |
| STAG3 | E117Q |
| STAG3 | D125G |
| STAG3 | D125Y |
| STAG3 | G129E |
| STAG3 | Q139K |
| STAG3 | C143Y |
| STAG3 | G145D |
| STAG3 | T148I |
| STAG3 | E170K |
| STAG3 | S183F |
| STAG3 | Y208C |
| STAG3 | G210D |
| STAG3 | S226L |
| STAG3 | S226* |
| STAG3 | R232S |
| STAG3 | P272S |
| STAG3 | R279W |
| STAG3 | R287L |
| STAG3 | R287C |
| STAG3 | D342N |
| STAG3 | T351I |
| STAG3 | H353Y |
| STAG3 | E358G |
| STAG3 | E358V |
| STAG3 | G369E |
| STAG3 | R374W |
| STAG3 | R386H |
| STAG3 | D389N |
| STAG3 | X389_splice |
| STAG3 | M394I |
| STAG3 | R408T |
| STAG3 | T422M |
| STAG3 | A444T |
| STAG3 | A444V |
| STAG3 | P455T |
| STAG3 | E456G |
| STAG3 | R460* |
| STAG3 | Q467Pfs*24 |
| STAG3 | R468C |
| STAG3 | R468C |
| STAG3 | R468C |
| STAG3 | R468C |
| STAG3 | A473T |
| STAG3 | D499E |
| STAG3 | A505V |
| STAG3 | R508Q |
| STAG3 | D511N |
| STAG3 | E513K |
| STAG3 | E536* |
| STAG3 | E536D |
| STAG3 | R543W |
| STAG3 | S546L |
| STAG3 | R554Q |
| STAG3 | G557R |
| STAG3 | H579L |
| STAG3 | L580I |
| STAG3 | S592L |
| STAG3 | P600S |
| STAG3 | Q603* |
| STAG3 | E619K |
| STAG3 | X621_splice |
| STAG3 | V632L |
| STAG3 | L642I |
| STAG3 | A644V |
| STAG3 | E656K |
| STAG3 | F660L |
| STAG3 | R662Q |
| STAG3 | V671L |
| STAG3 | L682P |
| STAG3 | E683K |
| STAG3 | L691V |
| STAG3 | E695D |
| STAG3 | X711_splice |
| STAG3 | R717C |
| STAG3 | Y721H |
| STAG3 | L727P |
| STAG3 | X741_splice |
| STAG3 | I753V |
| STAG3 | S761C |
| STAG3 | S771* |
| STAG3 | R774K |
| STAG3 | R776I |
| STAG3 | M777T |
| STAG3 | D790N |
| STAG3 | G817R |
| STAG3 | V826A |
| STAG3 | E830V |
| STAG3 | H844N |
| STAG3 | V845I |
| STAG3 | P849S |
| STAG3 | R866L |
| STAG3 | R866W |
| STAG3 | L867I |
| STAG3 | A875V |
| STAG3 | G876R |
| STAG3 | S892* |
| STAG3 | R921Q |
| STAG3 | K933T |
| STAG3 | P952T |
| STAG3 | E956K |
| STAG3 | R958K |
| STAG3 | R962W |
| STAG3 | S967T |
| STAG3 | Q971* |
| STAG3 | F1015I |
| STAG3 | S1016fs |
| STAG3 | S1016F |
| STAG3 | P1017H |
| STAG3 | R1018Q |
| STAG3 | H1021Y |
| STAG3 | C1034R |
| STAG3 | P1045T |
| STAG3 | W1046C |
| STAG3 | E1060D |
| STAG3 | E1064K |
| STAG3 | R1077H |
| STAG3 | G1080E |
| STAG3 | A1082V |
| STAG3 | P1084L |
| STAG3 | E1095G |
| STAG3 | I1102N |
| STAG3 | T1105K |
| STAG3 | R1116W |
| STAG3 | M1125I |
| STAG3 | E1126V |
| STAG3 | D1129Y |
| STAG3 | E1145K |
| STAG3 | R1148M |
| STAG3 | P1152T |
| STAG3 | M1176Rfs*9 |
| STAG3 | E1180G |
| STAG3 | E1181K |
| STAG3 | S1190A |
| STAG3 | D1198E |
| STAG3 | Q1200_splice |
| STAG3 | G1211E |
| STAG3 | L1215S |
| STAG3 | E1219K |
| STAG3 | D1221N |
| STAG3 | I1222T |
| STAG3 | D1224Y |

*STOP codon

Measuring Expression of Stag2 and/or Stag3

Methods to measure Stag2 and/or Stag3 gene expression products (e.g., protein and/or mRNA) are known to a skilled artisan. Such methods to measure gene expression products, e.g., protein level, include ELISA (enzyme linked immunosorbent assay), Western blot, immunoprecipitation, and immunofluorescence using detection reagents such as an antibody or protein binding agents.

For example, antibodies for Stag2 and/or Stag3 are commercially available and can be used to measure protein expression levels. Alternatively, since the amino acid sequences for Stag2 and/or Stag3 are known and publically available at NCBI website, one of skill in the art can raise their own antibodies against these proteins. As one of skill in the art can appreciate, binding of antibodies to a Stag2 and/or Stag3 protein can induce a conformational change in the structure and/or the function of the Stag2 and/or Stag3 protein. In one embodiment, antibodies that induce a conformational change or functional change in the Stag2 and/or Stag3 protein(s) are used in the methods and assays described herein.

In some embodiments, immunohistochemistry (“IHC”) and immunocytochemistry (“ICC”) techniques can be used to assay expression levels of Stag2 and/or Stag3. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that undergoes a biochemical reaction, and thereby experiences a change in color upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and is not described in detail herein. In other embodiments, protein samples are analyzed by mass spectroscopy.

Immunological tests can be used with the methods and assays described herein and include, for example, competitive and non-competitive assay systems using techniques such as Western blots, dot blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art. In some embodiment, the immunoassay can be a quantitative or a semi-quantitative immunoassay.

In certain embodiments, the gene expression products as described herein can be instead determined by detecting or measuring the level of messenger RNA (mRNA) expression of genes associated with the marker genes described herein. Such molecules can be isolated, derived, or amplified from a biological sample, such as a tumor biopsy. Detection of mRNA expression is known by persons skilled in the art, and includes, but is not limited to PCR procedures, RT-PCR, Northern blot analysis, differential gene expression, RNase protection assay, microarray analysis, hybridization methods, next-generation sequencing etc. Non-limiting examples of next-generation sequencing technologies can include Ion Torrent, Illumina, SOLID, 454; Massively Parallel Signature Sequencing; solid-phase, reversible dye-terminator sequencing; and DNA NANOBALL™ sequencing. In some embodiments, mRNA level of gene expression products described herein can be determined by reverse-transcription (RT) PCR and by quantitative RT-PCR (QRT-PCR) or real-time PCR methods. Methods of RT-PCR and QRT-PCR are known in the art. In one embodiment, an mRNA sequence is reverse-transcribed to a cDNA sequence, which alters the structure of the endogenous mRNA.

Other methods of detection for assessing expression of Stag2 and/or Stag3 include, but are not limited to, optical methods, electrochemical methods (e.g., voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

### Reference Value

The terms “reference value,” “reference level,” “reference sample,” and “reference” are used interchangeably herein and refer to the level of expression of a control marker (e.g., Stag2 and/or Stag3) in a known sample against which another sample (i.e., one obtained from a subject having, or suspected of having, cancer) is compared. A reference value is useful for determining the amount of expression of Stag2 and/or Stag3 or the relative increase/decrease of such expressional levels in a biological sample. A reference value serves as a reference level for comparison, such that samples can be normalized to an appropriate standard in order to infer the presence, absence or extent of Stag2/3 mutation and/or Stag2/3 expression levels (e.g., protein and/or mRNA levels).

In one embodiment, a biological standard is obtained at an earlier time point (e.g., prior to the onset of a cancer or prior to the onset of an anti-cancer treatment) from the same individual that is to be tested or treated as described herein. Alternatively, a standard can be from the same individual having been taken at a time after the onset or diagnosis of a cancer. In such instances, the reference value can provide a measure of the efficacy of treatment. It can be useful to use as a reference for a given patient a level from a sample taken after a cancer diagnosis but before the administration of any therapy to that patient. In one embodiment, the tissue type obtained for the reference sample is the same as the tissue type from which the tumor has originated.

Alternatively, a reference value can be obtained, for example, from a known biological sample from a different individual (e.g., not the individual being tested) that is e.g., substantially free of detectable cancer and/or known to be responsive to a BRAF inhibitor and/or in a phase of responsiveness to a BRAF inhibitor. A known sample can also be obtained by pooling samples from a plurality of individuals to produce a reference value or range of values over an averaged population, wherein a reference value represents an average level of expression of Stag2 and/or Stag3 as described herein among a population of individuals (e.g., a population of individuals substantially free of detectable cancer). Thus, the expression level of Stag2 and/or Stag3 in a reference value obtained in this manner is representative of an average level of the individual markers or combination of markers in a general population of individuals lacking cancer. An individual sample is compared to this population reference value by comparing expression of the Stag2 and/or Stag3 from a sample relative to the population reference value. Generally, an increase in the amount of expression over the reference value (e.g., a reference obtained from subjects lacking cancer) indicates that the cancer is/will be responsive to treatment with a BRAF inhibitor, while a decrease in the amount of expression indicates that the cancer is not sensitive or will not remain responsive to a BRAF inhibitor and a treatment comprising a PD-1, PD-L1, and/or ERK inhibitor should be employed instead. The converse is contemplated in cases where a reference value is obtained from a population of subjects having cancer. It should be noted that there is often variability among individuals in a population, such that some individuals will have higher levels of expression, while other individuals have lower levels of expression. However, one skilled in the art can make logical inferences on an individual basis regarding the detection and treatment of cancer as described herein.

In one embodiment, a reference sample can be non-tumor tissue derived from the individual having a cancer to be assessed using the methods described herein.

In one embodiment, a range of expression levels of Stag2 and/or Stag3 can be defined for a plurality of cancer-free subjects and/or for a plurality of subjects having cancer. Provided that the number of individuals in each group is sufficient, one can define a range of expression values for each population. These values can be used to define cut-off points for selecting a therapy or for monitoring progression of disease. Thus, one of skill in the art can determine an expression value and compare the value to the ranges in each particular sub-population to aid in determining the status of disease and the recommended course of treatment. Such value ranges are analogous to e.g., HDL and LDL cholesterol levels detected clinically. For example, LDL levels below 100 mg/dL are considered optimal and do not require therapeutic intervention, while LDL levels above 190 mg/dL are considered ‘very high’ and will likely require some intervention. One of skill in the art can readily define similar parameters for expression values in a cancer. These value ranges can be provided to clinicians, for example, on a chart, programmed into a PDA etc.

A standard comprising a reference value or range of values can also be synthesized. A known amount of Stag2 and/or Stag3 (or a series of known amounts) can be prepared within the typical expression range that is observed in a general cancer or cancer-free population. This method has an advantage of being able to compare the extent of disease in one or more individuals in a mixed population. This method can also be useful for subjects who lack a prior sample to act as a reference value or for routine follow-up post-diagnosis. This type of method can also allow standardized tests to be performed among several clinics, institutions, or countries etc.

In certain embodiments, the methods described herein further comprise a step to relate the communication of assay results or diagnoses or both to e.g., technicians, physicians or patients. In certain embodiments, computers can be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In one embodiment, a diagnosis based on the differential presence/absence of a mutation in Stag2/3, or expression levels of Stag2/3 a test subject is communicated to the subject or subject's clinician as soon as possible after the diagnosis is obtained. The diagnosis can be communicated to the subject by the subject's treating physician. Alternatively, the diagnosis can be sent to a test subject by email or communicated to the subject by phone. A computer can be used to communicate the diagnosis by email or phone. In certain embodiments, the message containing results of a diagnostic test can be generated and delivered automatically to the subject using a combination of computer hardware and software which are known in the art and not described herein.

### BRAF Inhibitors

The RAF protein family contains three members: BRAF, ARAF, and CRAF (also known as RAF-1). Each of the RAF proteins contains an amino-terminal regulatory domain, an activation loop, and a C-terminal kinase domain. The regulation of RAF involves phosphorylation of the regulatory and catalytic domains. Once activated, RAF molecules function as serine/threonine kinases capable of activating downstream signaling molecules by phosphorylation.

RAF is implicated in promoting cell proliferation by association with the mitogen-activated protein kinase (MAPK) signaling pathway. In particular, RAF proteins are the principle effectors of Ras-mediated signaling. Activated Ras interacts directly with RAF and recruits RAF to the cell membrane from the cytoplasm. Upon translocation to the cell membrane, Ras-bound RAF undergoes a series of phosphorylation events and conformational changes which serve to activate RAF serine/threonine kinase activity. RAF may also be activated through Ras-independent pathways involving interferon beta, protein kinase C (PKC) alpha, anti-apoptotic proteins such as Bcl-2, various scaffolding proteins, ultraviolet light, ionizing radiation, retinoids, erythropoietin, and dimerization between RAF isoforms.

Once activated, RAF mediates downstream signaling by phosphorylating the kinases MEK1 and MEK2, which contain a proline-rich sequence that enables recognition by RAF. BRAF is a far more potent activator of MEK1 and MEK2 than either ARAF or RAF-1. MEK1 and MEK2, in turn, phosphorylate and activate ERK1 and ERK2, which then translocate to the nucleus. Nuclear ERK1 and ERK2 activate transcription factors such as Elk-1, Fos, Jun, AP-1 and Myc, ultimately inducing transcription of genes involved in cell proliferation, dedifferentiation and survival, including, for example, cyclin D1, cyclin E, and cdc activator 25 phosphatase.

There are several BRAF inhibitors known currently including, but not limited to vemurafenib, dabrafenib, LGX818, sorafenib, PLX-4720, PDC-4032, GSK2118436, and PLX-3603 (also known as R05212054).

### Immune Checkpoint Inhibitors

The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals. In some embodiments, a subject or patient is treated with at least one inhibitor of an immune checkpoint protein.

Cytotoxic T-lymphocyte associated antigen 4 (CTLA-4) is an immune checkpoint protein that down-regulates pathways of T-cell activation (Fong et al., Cancer Res. 69 (2): 609-615, 2009; Weber Cancer Immunol. Immunother, 58:823-830, 2009). Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells, thereby blocking the negative down regulation of the immune responses elicited by the interaction of these molecules. Examples of anti-CTLA-4 antibodies are described in U.S. Pat. Nos. 5,811,097; 5, 811,097; 5,855,887; 6,051,227; 6,207,157; 6,682,736; 6,984,720; and 7,605,238. One anti-CDLA-4 antibody is tremelimumab, (ticilimumab, CP-675,206). In one embodiment, the anti-CTLA-4 antibody is ipilimumab (also known as 10 D1, MDX-D010) a fully human monoclonal IgG antibody that binds to CTLA-4. Ipilimumab is marketed under the name YERVOY™ and has been approved for the treatment of unresectable or metastatic melanoma.

Further examples of checkpoint molecules that can be targeted for blocking or inhibition include, but are not limited to, PDL2, B7-H3, B7-H4, BTLA, HVEM, GAL9, VISTA, KIR, 2B4 (belongs to the CD2 family of molecules and is expressed on all NK, γδ, and memory CD8+ (αβ) T cells), CD160 (also referred to as BY55), A2aR, TIGIT, DD1-α. TIM-3. Lag-3, and various B-7 family ligands. B7 family ligands include, but are not limited to, B7-1, B7-2, B7-DC, B7-H1, B7-H2, B7-H3, B7-H4, B7-H5, B7-H6 and B7-H7.

Another immune checkpoint protein is programmed cell death 1 (PD-1). PD-1 limits the activity of T cells in peripheral tissues at the time of an inflammatory response to infection and limits autoimmunity. PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll. Nature Reviews Cancer. 12:252-264, 2012). PD1 blockade can be accomplished by a variety of mechanisms including antibodies that bind PD1 or its ligand. PD-L1. Examples of PD-1 and PD-L1 blockers are described in U.S. Pat. Nos. 7,488,802; 7, 943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In certain embodiments the PD-1 blockers include anti-PD-L1 antibodies. In certain other embodiments the PD-1 blockers include anti-PD-1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-224, a fusion protein of B7-DC; an antibody Fc portion; BMS-936559 (MDX-1105-01) for PD-L1 (B7-H1) blockade. Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207: 2175-86 and Sakuishi et al., 2010, J. Exp. Med. 207: 2187-94).

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

Also specifically contemplated herein are agents that disrupt or block the interaction between PD-1 and PD-L1, such as a high affinity PD-L1 antagonist.

MEK and/or ERK Inhibitors

In some embodiments of the methods described herein, an ERK inhibitor is used for treating a subject having cancer. ERK is the only known substrate for MEK1 and MEK2. Phosphorylation of ERK results in translocation to the nucleus where it phosphorylates nuclear targets and regulates various cellular processes such as proliferation, differentiation, and cell cycle progression (J. L. Yap et al., Chem. Med. Chem. 2011 6:38).

The term “ERK inhibitors” as used herein relates to compounds capable of fully or partially preventing, or reducing or inhibiting ERK1/2 signaling activity. Inhibition may be effective at the transcriptional level, for example by preventing or reducing or inhibiting mRNA synthesis of ERK1 or ERK2 mRNA, for example, human ERK1 (NCBI reference NP-002737) or human ERK2 (NCBI reference NP-620407). Exemplary small molecule ERK inhibitors include, but are not limited to SCH772984, 3-(2-aminoethyl)-5-)) 4-ethoxyphenyl)methylene)-2,4-thiazolidinedione (PKI-ERK-005), CAY10561 (CAS 933786-58-4: CAYMAN CHEMICAL), and VTXX11e.

As used herein, the term “MEK inhibitors” refers to compounds capable of fully or partially preventing or reducing or inhibiting MEK signaling activity. Inhibition may be effective at the transcriptional level, for example, by preventing or reducing or inhibiting mRNA synthesis of mRNA encoding human MEK1 (NCBI reference NP-002746), or human MEK2 (NCBI reference NP109587). Exemplary small molecule inhibitors of MEK include, but are not limited to PD 98059, a highly selective inhibitor of MEK1 and MEK2 with IC50 values of 4 μM and 50 μM respectively (Runden E et al., J Neurosci 1998, 18 (18) 7296-305), trametinib (GSK 120212), cobimetinib (XL518), MEK 162, RO5126766, GDC-0623, PD0325901 (Pfizer), Selumetinib, a selective MEK inhibitor (Astrazeneca/Array Biopharma, also known as AZD6244), ARRY-438162 (Array Biopharma), PD198306 (Pfizer), AZD8330 (Astrazeneca/Array Biopharma, also called ARRY-424704), PD184352 (Pfizer, also called CI-1040), PD 184161 (Pfizer), α-[Amino[(4-aminophenyl)thio]methylene]-2-(trifluoromethyl)benzeneacetonitrile (SL327), 1,4-Diamino-2,3-dicyano-1,4-bis(2-aminophenylthio) butadiene (U0126), Ro 09-2210 (Roche), RDEA1 19 (Ardea Biosciences), and ARRY-704 (Astrazeneca).

Also contemplated are combination treatments for use in the methods described herein, the treatments comprising an ERK inhibitor and a MEK inhibitor. Without wishing to be bound by theory, this combination treatment has been shown to be effective in experiments with naïve K-ras mutant cells where MEK and ERK inhibitors inhibited the out-growth of resistant cells, whereas ERK inhibitor treatment of cells with acquired MEK inhibitor resistance effectively blocked proliferation (G Hatzivassiliou et al., Mol. Cancer Ther. 2012 11:1143-1154). Such combination treatments are described in e.g., U.S. patent application No. 20150111869.

It is important to note that cancers can be sensitive to ERK inhibitors while being resistant to MEK inhibitors. For example, Stag2 mutant melanomas are resistant to MEK inhibitors, but are sensitive to ERK inhibitors. Although ERK is downstream of MEK, experiments as shown in the Examples section indicate that melanoma patients can be resistant to either monotherapy using a BRAF inhibitor of MEK inhibitor, as well as to the combination of BRAF/MEK inhibitors, which is now standard.

### Other Inhibitors of the BRAF-MEK-ERK Pathway

Also specifically contemplated herein are inhibitors that inhibit or reduce the function of signaling pathway members upstream of ERK. Any of these upstream elements, if targeted, can also cause resistance that can be compensated by providing an ERK inhibitor. Exemplary pathway members include, but are not limited to, Ras, NF1, RASGAP1, RASGAP2, SPRY, GRB2, SOS, PAK1, KSR1, and KSR2.

Exemplary Ras kinase inhibitors include, for example, BMS-214662 (Bristol-Meyers Squibb), SCH 66336 (also known as Ionafarnib: Schering-Plough), L-778,123 (Merck), R115777 (also known as ZARNESTRA™ or Tipifarnib: Johnson & Johnson), and 6-[(4-chloro-phenyl)-hydroxy-(3-methyl-3H-imidazol-4-yl)-methyl]-4-(3-ethynyl-phenyl)-1-methyl-1H-quinolin-2-one (Osi Pharmaceuticals, Inc.). Additional Ras inhibitors are known to those of skill in the art and are not described in detail herein. Similarly, inhibitors of NF1, RASGAP1, RASGAP2, SPRY, GRB2, SOS, PAK1, KSR1, and KSR2 are known to those of skill in the art and are not described herein.

### Dosage and Administration

In some aspects, the methods described herein provide a method for selecting an anti-cancer agent for treating cancer in a subject. In one embodiment, the subject can be a mammal. In another embodiment, the mammal can be a human, although the approach is effective with respect to all mammals. The methods comprise administering to the subject an effective amount of a pharmaceutical composition comprising an inhibitor of BRAF, PD-1, PD-L1, ERK or a combination thereof in a pharmaceutically acceptable carrier.

The dosage range for the agent depends upon the potency, and includes amounts large enough to produce the desired effect, e.g., treatment of cancer. The dosage should not be so large as to cause unacceptable adverse side effects. Generally, the dosage will vary with the type of inhibitor (e.g., an antibody or fragment, small molecule, siRNA, etc.), and with the age, condition, and sex of the patient. The dosage can be determined by one of skill in the art and can also be adjusted by the individual physician in the event of any complication.

Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in expression of a cancer biomarker (see “Efficacy Measurement” below). Such effective amounts can be gauged in clinical trials as well as animal studies for a given agent.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., a tumor). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Pharmaceutical Compositions

The present disclosure includes, but is not limited to, therapeutic compositions, such as inhibitors of BRAF, PD-1, PD-L1, MEK, and/or ERK, that are useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

### Efficacy Measurement

The efficacy of a given treatment for a cancer (including, but not limited to, breast cancer, melanoma etc.) can be determined by the skilled clinician. However, a treatment is considered “effective treatment,” as the term is used herein, if any one or all of the signs or symptoms of the cancer is/are altered in a beneficial manner, or other clinically accepted symptoms or markers of disease are improved, or ameliorated, e.g., by at least 10% following treatment with an agent that comprises an inhibitor of BRAF, PD-1, PD-L1, MEK and/or ERK. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the cancer; or (2) relieving the disease, e.g., causing regression of symptoms; and (3) preventing or reducing the likelihood of the development of the disease, or preventing secondary diseases/disorders associated with the cancer (e.g., cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., pain, tumor size, tumor growth rate, blood cell count, etc.

In some embodiments, the subject is further evaluated using one or more additional diagnostic procedures, for example, by medical imaging, physical exam, laboratory test(s), clinical history, family history, gene test, BRCA test, and the like. Medical imaging is well known in the art. As such, the medical imaging can be selected from any known method of imaging, including, but not limited to, ultrasound, computed tomography scan, positron emission tomography, photon emission computerized tomography, and magnetic resonance imaging.

The present invention may be as defined in any one of the following numbered paragraphs:
1. A method of selecting a treatment for cancer, the method comprising: measuring the activity and/or expression levels of STAG2 and/or STAG3 in a biological sample obtained from a subject having or suspected of having cancer, wherein if the activity and/or expression levels are substantially similar or increased compared to a reference, administration of a treatment comprising a BRAF inhibitor is selected, and wherein if the activity and/or expression levels are decreased compared to a reference, administration of a treatment is selected from the group consisting of: a PD-1 inhibitor, a PD-L1 inhibitor, and an ERK inhibitor.
2. The method of paragraph 1, further comprising a step of selecting a subject having a cancer comprising a mutation in BRAF, STAG2 and/or STAG3.
3. The method of paragraph 1 or 2, wherein the mutation in BRAF is V600E or V600K.
4. The method of paragraph 1, 2, or 3, wherein the mutation in STAG2 is a loss-of-function mutation.
5 The method of any one of paragraphs 1-4, wherein the loss-of-function mutation is D193N or K1083*.
6. The method of any one of paragraphs 1-5, wherein the BRAF inhibitor is vemurafenib, dabrafenib, LGX818, sorafenib or PLX-4720.
7. The method of any one of paragraphs 1-6, wherein the BRAF inhibitor is administered in combination with a MEK inhibitor.
8. The method of any one of paragraphs 1-7, wherein the MEK inhibitor is trametinib, cobimetinib, MEK162, AZD6244, RO5126766, or GDC-0623.
9. The method of any one of paragraphs 1-8, wherein the ERK inhibitor is SCH772982 or VTX11e.
10. The method of any one of paragraphs 1-9, wherein the PD-1or PD-L1 inhibitor is nivolumab, pembrolizumab, pidilizumab, BMS-936559, or MPDL-3280A.
11. The method of any one of paragraphs 1-10, wherein the reference is the activity and/or expression of STAG2 and/or STAG3 in a population of subjects having a cancer known to be responsive to a BRAF inhibitor or having a cancer in a phase of responsiveness to a BRAF inhibitor.
12. The method of any one of paragraphs 1-11, wherein the cancer is selected from the group consisting of: non-Hodgkin lymphoma, colorectal cancer, melanoma, papillary thyroid carcinoma, non-small-cell lung carcinoma, and adenocarcinoma of the lung.
13. The method of any one of paragraphs 1-12, further comprising a step of administering the selected treatment to a subject.
14. The method of any one of paragraphs 1-13, wherein the biological sample comprises a blood sample, a serum sample, a circulating tumor cell sample, a tumor biopsy, or a tissue sample.
15. The method of any one of paragraphs 1-14, wherein the measuring step comprises contacting the biological sample with an antibody that specifically binds to STAG2 and/or STAG3.
16. The method of any one of paragraphs 1-15, wherein the mutation in BRAF, STAG2 and/or STAG3 is identified by a DNA sequencing method.
17. The method of any one of paragraphs 1-16, wherein the DNA sequencing method comprises real-time PCR, Sanger sequencing, pyrosequencing, a TH×ID BRAF mutation test, a COBAS® BRAF mutation test, and bidirectional direct sequencing.
18. A method of monitoring a subject for the development of cancer resistance to a BRAF inhibitor, the method comprising: measuring the activity and/or expression levels of STAG2 and/or STAG3 in a sample obtained from a subject being treated with a BRAF inhibitor, wherein if the activity and/or expression levels are substantially similar or increased compared to the activity and/or expression levels prior to the onset of treatment with a BRAF inhibitor, the subject is determined to have a cancer that is sensitive to the BRAF inhibitor, and wherein if the activity and/or expression levels are decreased compared to the activity and/or expression levels prior to the onset of treatment with a BRAF inhibitor, the subject is determined to have a cancer that is resistant to or is developing resistance to a BRAF inhibitor.
19. The method of paragraph 18, wherein the subject having a cancer determined to be resistant to or developing resistance to a BRAF inhibitor is treated with an ERK inhibitor, a PD-1 inhibitor or a PD-L1 inhibitor.
20. The method of paragraph 18 or 19, wherein the ERK inhibitor is SCH772982 or VTX11e.
21. The method of paragraph 18, 19, or 20, wherein the PD-1or PD-L1 inhibitor is nivolumab, pembrolizumab, pidilizumab, BMS-936559, or MPDL-3280A.
22. The method of any one of paragraphs 18-21, wherein the BRAF inhibitor is vemurafenib, dabrafenib, LGX818, sorafenib or PLX-4720.
23. The method of any one of paragraphs 18-22, wherein the BRAF inhibitor is administered in combination with a MEK inhibitor.
24. The method of any one of paragraphs 18-23, wherein the MEK inhibitor is trametinib, cobimetinib, MEK162, AZD6244, RO5126766, and GDC-0623.
25. The method of any one of paragraphs 18-24, further comprising a step of selecting a subject having a cancer comprising a mutation in BRAF, STAG2 and/or STAG3.
26. The method of any one of paragraphs 18-25, wherein the mutation in BRAF is V600E or V600K.
27. The method of any one of paragraphs 18-26, wherein the mutation in STAG2 is a loss-of-function mutation.
28. The method of any one of paragraphs 18-27, wherein the loss-of-function mutation is D193N or K1083*.
29. The method of any one of paragraphs 18-28, wherein the subject was diagnosed with a cancer selected from the group consisting of: non-Hodgkin lymphoma, colorectal cancer, melanoma, papillary thyroid carcinoma, non-small-cell lung carcinoma, and adenocarcinoma of the lung.
30. The method of any one of paragraphs 18-29, wherein the biological sample comprises a blood sample, a serum sample, a circulating tumor cell sample, a tumor biopsy, or a tissue sample.
31. The of any one of paragraphs 18-30, wherein the measuring step comprises contacting the biological sample with an antibody that specifically binds to STAG2 and/or STAG3.
32. The method of any one of paragraphs 18-31, wherein the mutation in BRAF, STAG2 and/or STAG3 is identified by a DNA sequencing method.
33. The method of any one of paragraphs 18-32, wherein the DNA sequencing method comprises real-time PCR, Sanger sequencing, pyrosequencing, a TH×ID BRAF mutation test, a COBAS® BRAF mutation test, and bidirectional direct sequencing.

### EXAMPLES

### Example 1: Loss of Stag2/Stag3 Confers Resistance to BRAF Inhibition in Melanoma

BRAF is a major oncogenic driver and therapeutic target in melanoma. Inhibitors of BRAF, such as vemurafenib and dabrafenib, have shown high response rates and improved survival in melanoma patients with BRAF Val600 mutations, but a vast majority of these patients develop drug resistance^{1,2}. Several genetic mechanisms have been described in melanomas resistant to BRAF inhibitors (BRAFi), including those altering the MAPK pathway (NRAS, BRAF, MAP2K1/2, MITF and NF1) and the PI3K/Akt pathway (PIK3CA, PIK3R1 PTEN and Akt)³⁻⁸. However, there is still a significant portion (18%-26%) of BRAFi resistant melanoma tumors that are not driven by any of these known resistance mechanisms^{4,5,9}. The data provided herein show that loss of Stromal antigen 2 or 3 (Stag2 or Stag3), which encode subunits of the cohesin complex^{10,11}, resulted in resistance to BRAF inhibitors in melanoma. Loss-of-function mutations in Stag2 and decreased expression of Stag2/3 proteins were observed in tumor samples from patients developed resistance to BRAFi and in BRAFi-resistant melanoma cell lines. Knockdown of Stag2 or Stag3 decreased sensitivity to BRAFi^{V600EGLU}-mutant melanoma cells and xenograft tumors to BRAFi. Loss of Stag2 inhibited CCCTC-binding-factor-mediated expression of dual specificity phosphatase 6 (DUSP6), leading to reactivation of mitogen-activated protein kinase (MAPK) signaling (via the MAPKs ERK1 and ERK2; hereafter referred to as ERK). These studies unveil a previously unknown genetic mechanism of BRAFi resistance and provide new insights into the tumor suppressor function of STAG2 and STAG3.

Several genetic mechanisms mediating resistance to BRAFi have been described, including mutations in genes encoding components of the MAPK pathway (NRAS, MAP2K1, MAP2K2 and NF1) and the phosphatidylinositol-4,5-bisphosphate 3-kinase (PI3K)-protein kinase B (PKB, also known as AKT) pathway (PIK3CA, PIK3R1, PTEN and AKT)^{3,8}. However, a portion (18-26%) of BRAFi-resistant melanomas are not driven by any of these known resistance mechanisms^{4,5,9}.

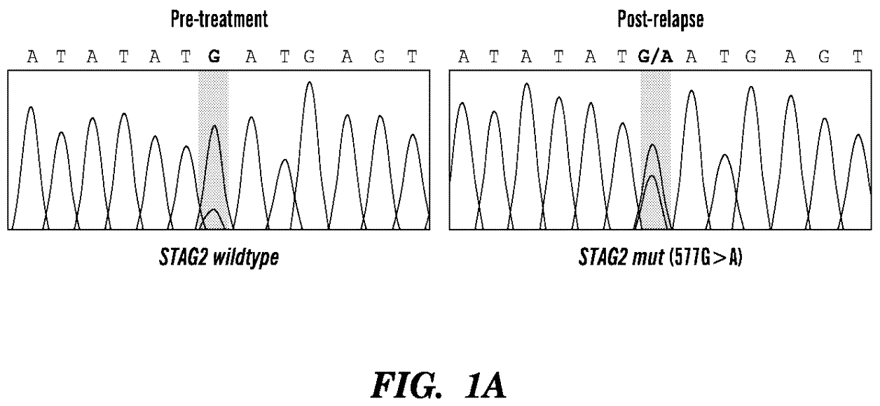

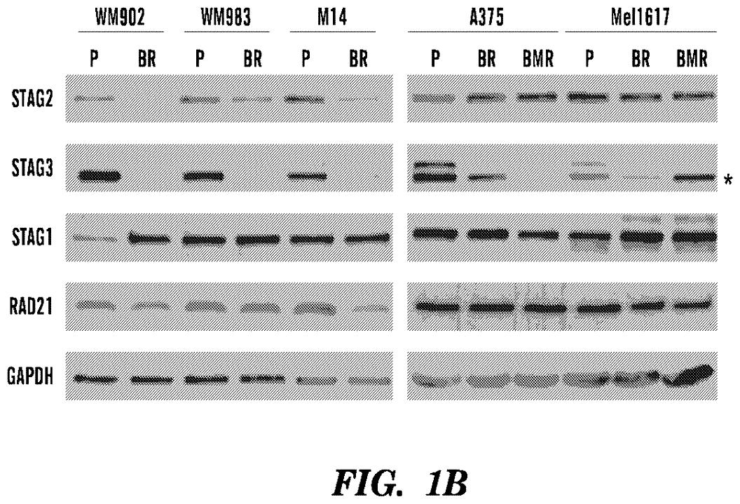

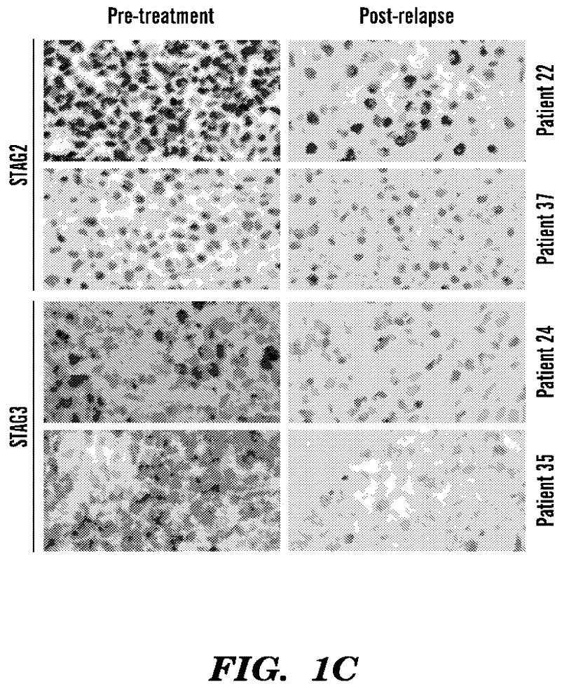

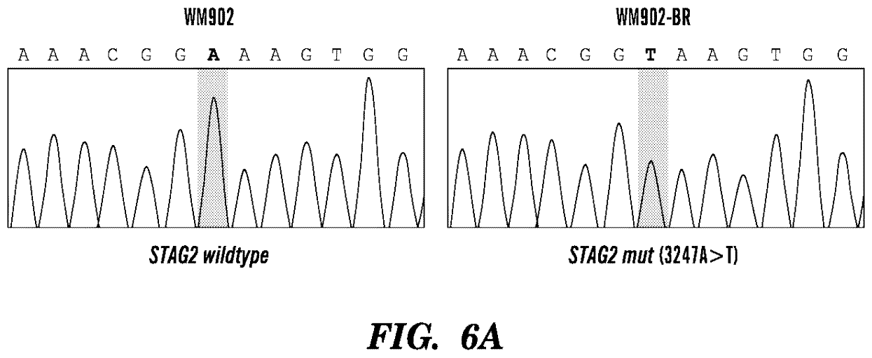

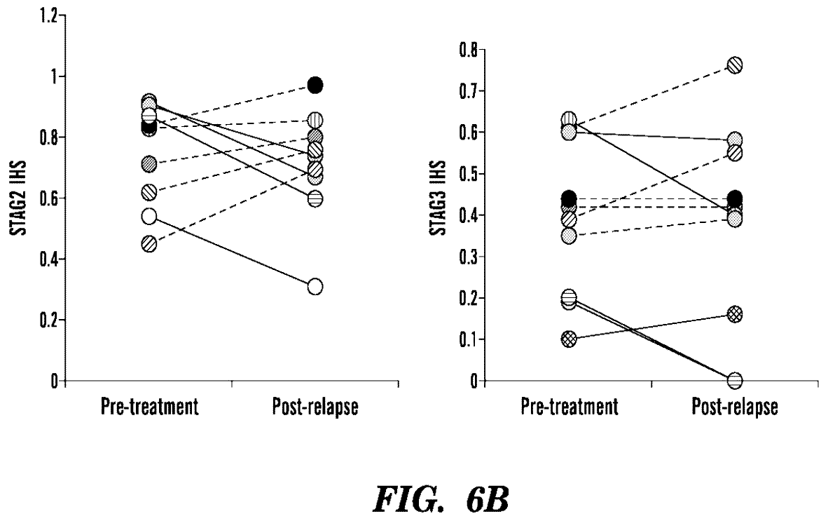

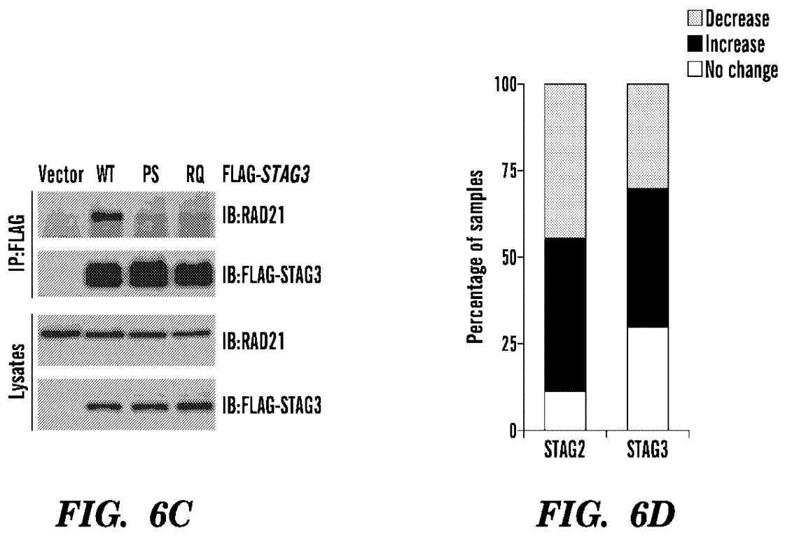

To identify additional mechanisms of acquired resistance to BRAF inhibition, whole-exome sequencing was performed on a pair of pre-treatment and post-relapse melanoma tumor samples from a patient who was treated with the BRAFi vemurafenib and who had a time-to-disease-progression of 5 months. The list of mutations identified exclusively in the post-relapse sample from this patient were compared with the 127 significantly mutated genes (SMG) identified from The Cancer Genome Atlas (TCGA) Pan-cancer analysis¹⁰), and it was found that there was only one SMG (STAG2) that was mutated in the post-relapse sample. This mutation in STAG2 gene (c.577G>A, resulting in Asp193Asn) was subsequently confirmed by Sanger sequencing. Although the pretreatment sample contains trace amounts of the mutated allele, it is greatly enriched in the post-relapse sample (FIG. 1A). STAG2 (also known as SA2) encodes a core subunit in the cohesin complex that regulates cohesion and segregation of sister chromatids^{11,12}. Mutations in STAG3 and other cohesin complex subunits (such as SMC1A, SMC3 and RAD21) have been shown to occur frequently in various cancers, such as urothelial bladder carcinomas, Ewing sarcoma, acute myeloid leukemia, myelodysplastic syndrome and acute megakaryoblastic leukemia¹³⁻²³. It was determined that the Stag 2^{Asp193Asn} mutant decreases the binding affinity of the STAG2 to Rad21 and SMC1A, indicating that c.577G>A is a loss-of-function mutation. Stag2 has two other paralogs in mammals, Stag1 and Stag3. Data from the melanoma TCGA project²⁴ indicated that mutation frequencies of these three proteins are ˜4% (STAG2), 3% (STAG3) and 5% (STAG3), with a total non-redundant mutation rate of ˜10%. The expression of all three STAG proteins was examined in a panel of melanoma cell lines that acquired resistance after chronic exposure to BRAFi^{25 26} and it was found that both STAG2 and STAG3, but not Stag1, protein levels were reduced in several BRAFi-resistant (BR) cell lines and in BRAFi/MEKi double-resistant (BMR) lines as compared to their drug-sensitive counterparts (FIG. 1B). Sanger sequencing was subsequently performed for all coding exons of STAG2 and STAG3 genes in these cell line pairs and identified a STAG2 nonsense mutation (c.3247A>T, resulting in a change of Lys1083 to a stop codon (Lys1083*)) in WM902BR cells, which was not present in the parental WM902 cells (FIG. 6A). No mutations in STAG3 were identified in this cell line panel. However, when data were analyzed from a published whole-exome sequencing study of 45 patients with BRAF^{Val600}-mutant metastatic melanoma who received vemurafenib or dabrafenib monotherapy⁴, it was found that three STAG3 mutations in pre-treatment samples from 14 patients who developed early resistance to therapy (<12 weeks: Table 2). STAG3 mutations were detected in post-relapse, but not pre-treatment, samples from an additional six patients from this study (Table 2).

TABLE 2B
List of mutations identified exclusively in the post-relapse sample,
but not the pre-treatment sample, from a melanoma patient who was
treated with vemurafenib and relapsed with a time to disease progression
of 5 months.

| | | | | | Function | |
|---|---|---|---|---|---|---|
| Chr | Start | End | Ref | Alt | Ref. | Gene Ref. |
| chrX | 1.23E+08 | 1.23E+08 | G | A | exonic | STAG2 |
| chr15 | 42439924 | 42439924 | T | G | exonic | PLA2G4F |
| chr5 | 1.42E+08 | 1.42E+08 | T | G | exonic | ARHGAP26 |
| chr5 | 1.54E+08 | 1.54E+08 | T | G | exonic | GALNT10 |
| chr16 | 720991 | 720991 | A | C | exonic | RHOT2 |
| chr4 | 96124082 | 96124082 | T | G | exonic | UNC5C |
| chr17 | 32962000 | 32962000 | T | G | exonic | TMEM1323 |
| chr22 | 20819608 | 20819608 | C | G | exonic | KLHL22 |
| chr15 | 81229165 | 81229165 | A | C | exonic | CEMIP |
| chr21 | 33074197 | 33074197 | T | G | exonic | SCAF4 |
| chr2 | 2.16E+08 | 2.16E+08 | A | G | exonic | ABCA12 |
| chr6 | 2685596 | 2685596 | T | A | exonic | MYLK4 |
| chr16 | 27460543 | 27460543 | A | C | exonic | IL21R |
| chr1 | 22199894 | 22199894 | T | G | exonic | HSPG2 |
| chr15 | 41029893 | 41029893 | G | T | exonic | RMDN3 |
| chr10 | 73767576 | 73767576 | G | C | exonic | CHST3 |
| chr3 | 58145336 | 58145336 | C | A | exonic | FLNB |
| chr10 | 72061204 | 72061204 | T | G | exonic | LRRC20 |
| chrX | 1.53E+08 | 1.53E+08 | G | T | exonic | AVPR2 |
| chr11 | 1.19E+08 | 1.19E+08 | A | C | exonic | DPAGT1 |
| chr5 | 1.49E+08 | 1.49E+08 | T | G | exonic | IL17B |
| chr13 | 37012872 | 37012872 | T | G | exonic | CCNA1 |
| chr21 | 27372378 | 27372378 | T | C | exonic | APP |
| chr9 | 22006176 | 22006176 | T | G | exonic | CDKN2B |
| chr14 | 23313599 | 23313599 | T | G | exonic | MMP14 |
| chr1 | 1.1E+08 | 1.1E+08 | A | C | exonic | AMPD2 |
| chr1 | 1.51E+08 | 1.51E+08 | C | G | exonic | RFX5 |
| chr9 | 1.04E+08 | 1.04E+08 | C | T | exonic | GRIN3A |
| chr5 | 1.73E+08 | 1.73E+08 | T | G | exonic | NKX2-5 |
| chr9 | 96060194 | 96060194 | A | C | exonic | WNK2 |
| chr1 | 47283669 | 47283669 | C | T | exonic | CYP4B1 |
| chr11 | 66033214 | 66033214 | G | C | exonic | KLC2 |
| chr17 | 31618750 | 31618750 | T | G | exonic | ASIC2 |
| chr11 | 76796018 | 76796018 | A | C | exonic | CAPN5 |
| chr16 | 20651865 | 20651865 | T | C | exonic | ACSM1 |
| chr2 | 1.56E+08 | 1.56E+08 | A | C | exonic | KCNJ3 |
| chr12 | 58124709 | 58124709 | C | T | exonic | AGAP2 |
| chr11 | 1.26E+08 | 1.26E+08 | A | C | exonic | DCPS |
| chr16 | 2522806 | 2522806 | A | C | exonic | NTN3 |
| chr19 | 18119220 | 18119220 | T | G | exonic | ARRDC2 |
| chr2 | 856929027 | 856929027 | A | C | exonic | CAPG |
| chr1 | 2.27E+08 | 2.27E+08 | A | C | exonic | PARP1 |
| chr1 | 26515317 | 26515317 | T | G | exonic | CNKSR1 |
| chr9 | 15422971 | 15422971 | T | G | exonic | SNAPC3 |
| chr20 | 43726317 | 43726317 | C | T | exonic | LCM51 |
| chr13 | 1.11E+08 | 1.11E+08 | G | A | exonic | COL4A2 |
| chr12 | 1.33E+08 | 1.33E+08 | A | C | exonic | EP400 |
| chr12 | 51868963 | 51868963 | T | A | exonic | SLC4A8 |
| chr1 | 1.87E+08 | 1.87E+08 | A | C | exonic | PLA2G4A |
| chr19 | 13136208 | 13136208 | T | G | exonic | NFIX |
| chr11 | 1.2E+08 | 1.2E+08 | A | G | exonic | OAF |
| chr2 | 27707975 | 27707975 | A | C | exonic | IFT172 |
| chr13 | 52518307 | 52518307 | C | T | exonic | ATP7B |
| chr22 | 19213138 | 19213138 | C | A | exonic | CLTCL1 |
| chr6 | 38816525 | 38816525 | T | G | exonic | DNAH8 |
| chrX | 1.06E+08 | 1.06E+08 | A | T | exonic | TBC1D88 |
| chr8 | 85686850 | 85686850 | C | A | exonic | RALYL |

TABLE 3
Stag3 mutations

| PATIENT | Early | |
|---|---|---|
| # | Resistance | Mutation |
| 4 | yes | P272S (pre-treatment) |
| 26 | yes | R508Q (pre-treatment) |
| 40 | no | A107fs (post-relapse) |
| 41 | no | A644V, A1082V (post-relapse) |
| 45 | no | p.G129E (post-relapse) |
| 46 | yes | E1064K (pre-treatment) |
| 51 | no | E683K (post-relapse) |
| 60 | yes | S1016fs (post-relapse) |
| 63 | no | D30N, D1221N (post-relapse) |

Although the significance of STAG3 mutations was not reported in the original study⁴, it was found that two of these mutations reduced the binding affinity of STAG3 to RAD21 (FIG. 6C). Finally, expression of Stag2 and Stag3 proteins was compared, using immunohistochemical analysis, in pairs of pre-treatment and post-relapse tumor samples from patients who had been treated with BRAFi monotherapy or BRAFi/MEKi combination therapy. Four and three post-relapse samples, respectively, of a total of nine pairs of samples, showed decreased levels of STAG2 and STAG3 proteins, relative to their paired pretreatment samples (FIG. 1C & FIG. 6D). Two of these samples showed reductions in both STAG2 and STAG3 expression, and without wishing to be bound by theory indicates that their down-regulation was mediated through epigenetic mechanisms. Taken together, these results indicate that mutations in STAG2 and STAG3 that decrease expression of their proteins are involved in clinical development of BRAFi resistance in patients with melanoma.

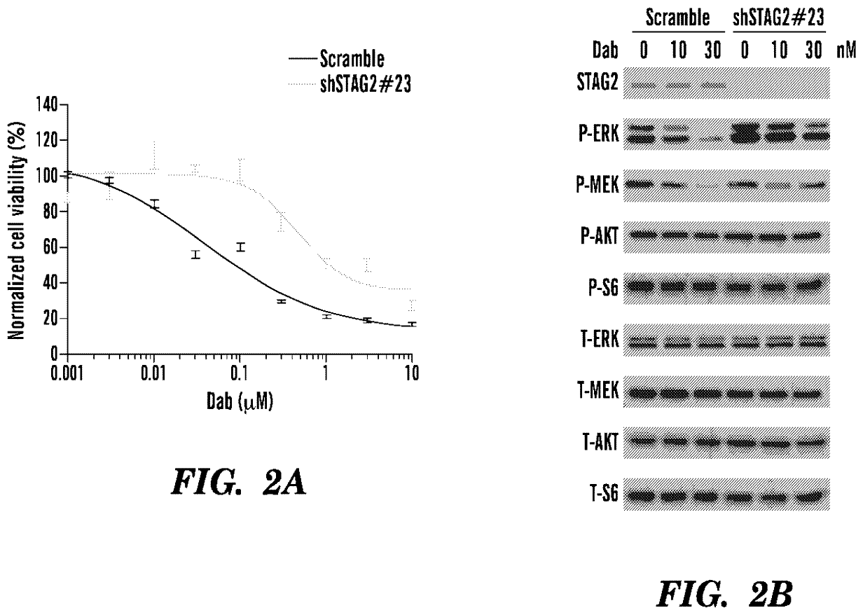

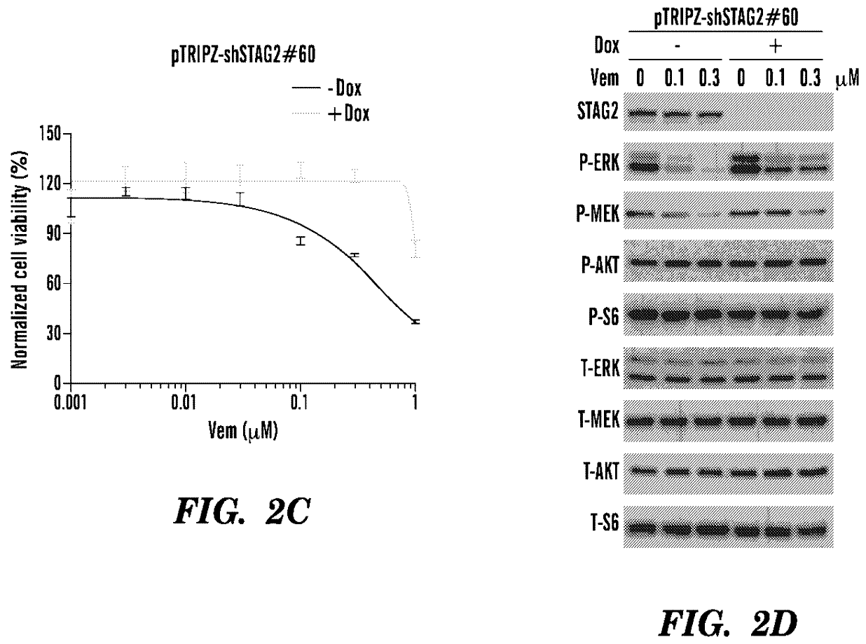

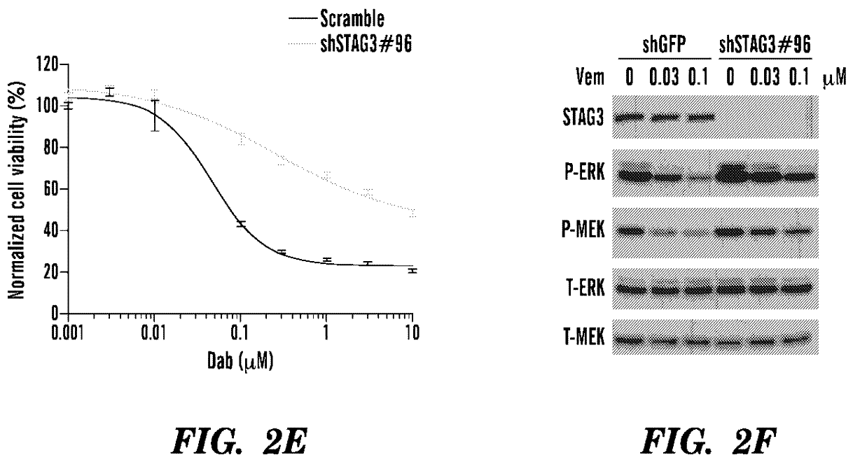

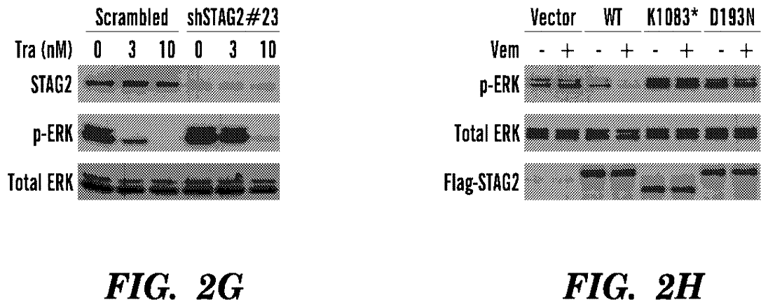

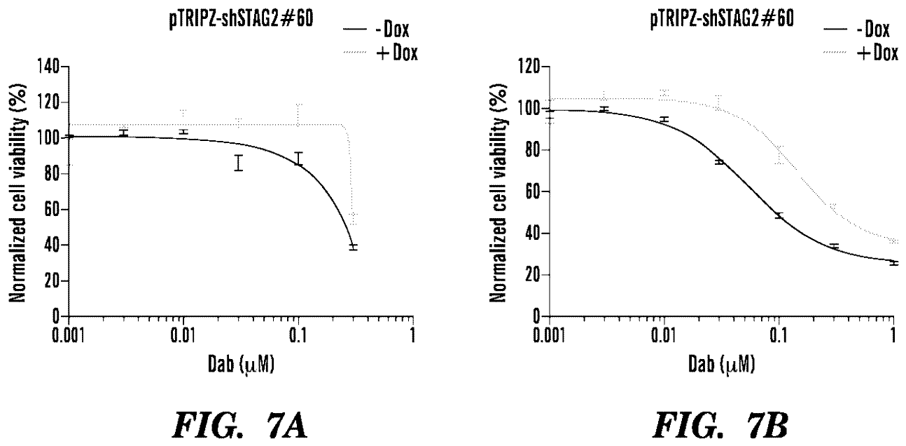

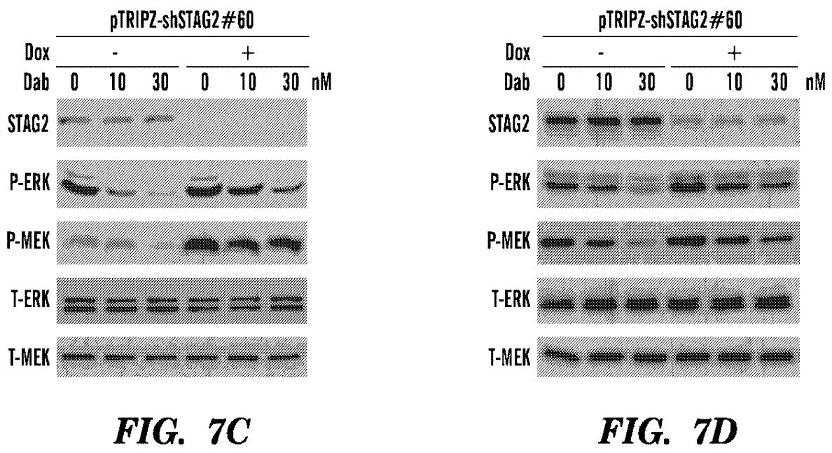

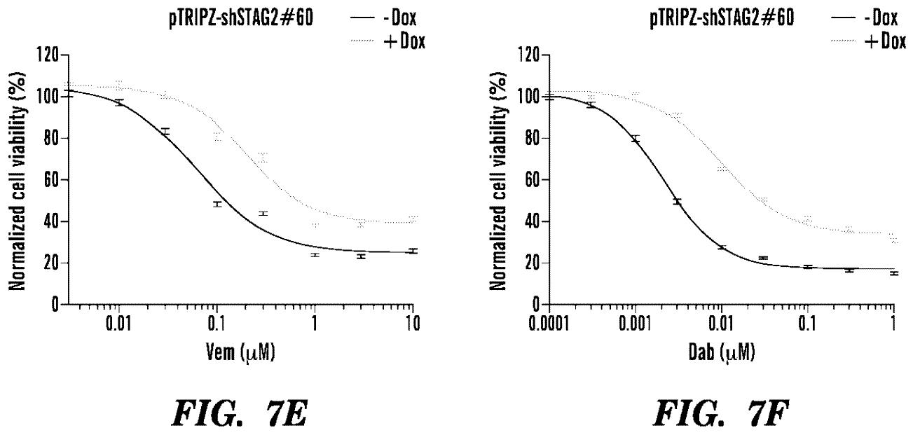

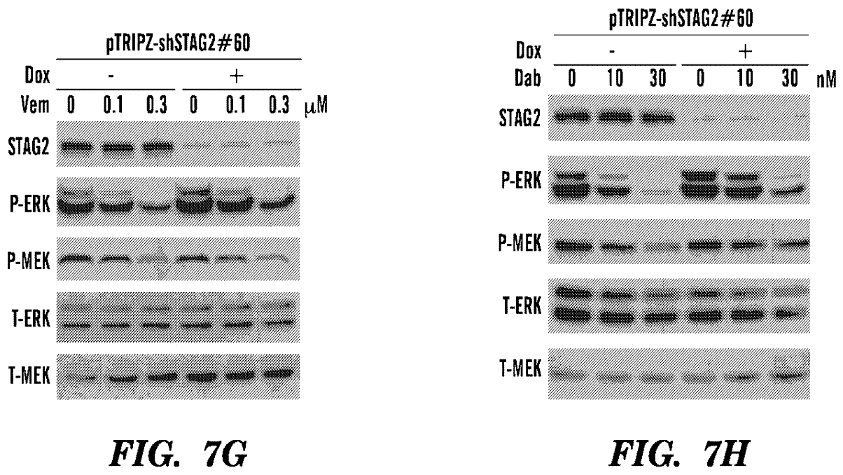

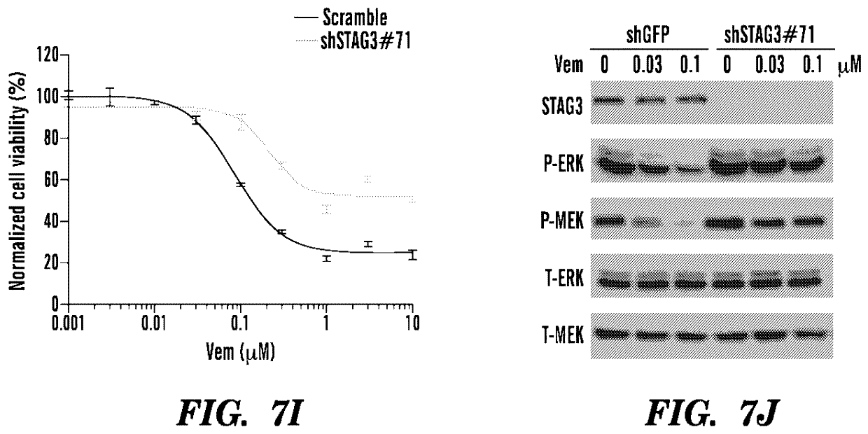

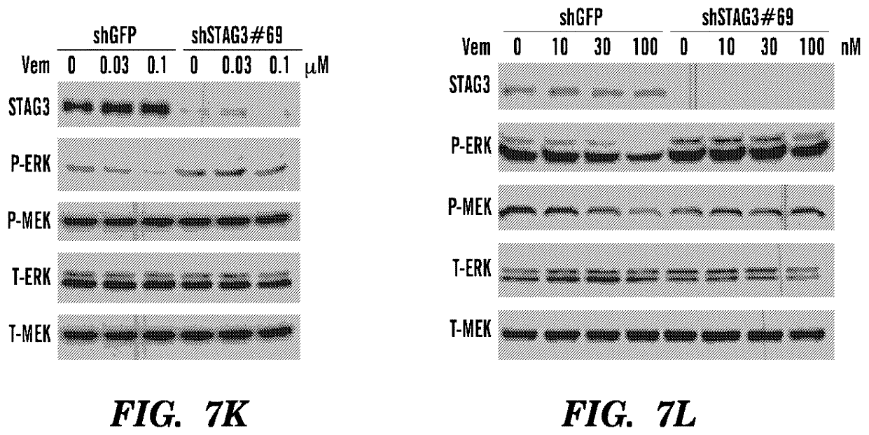

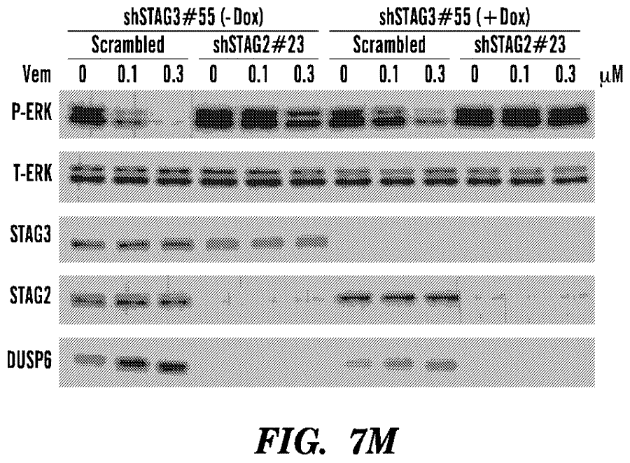

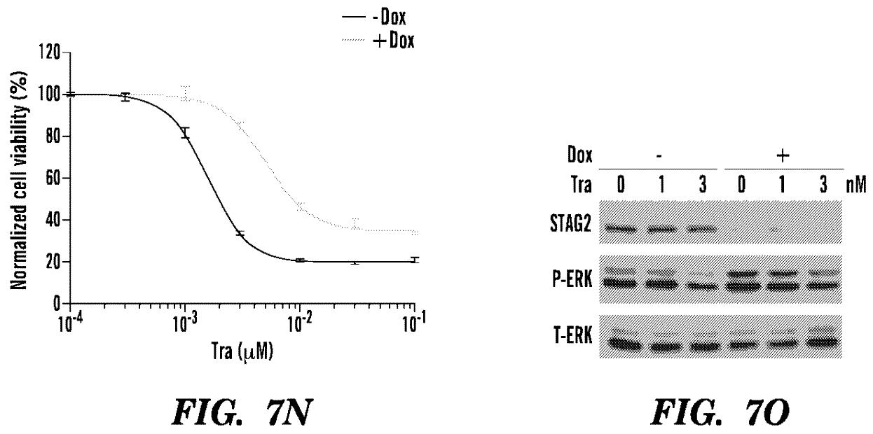

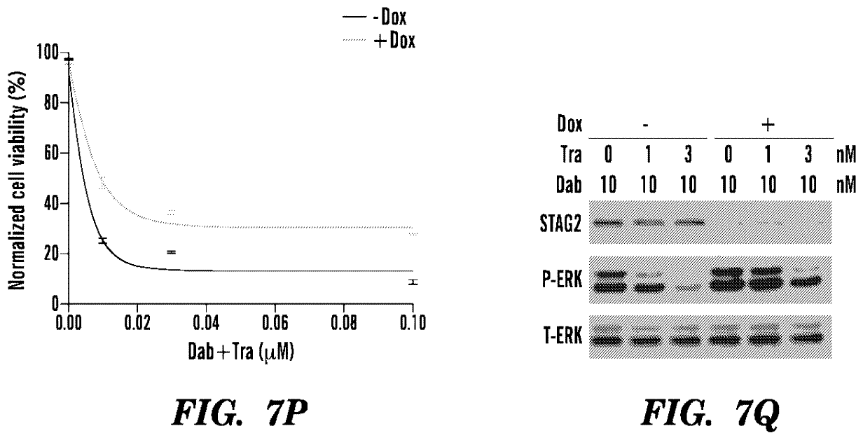

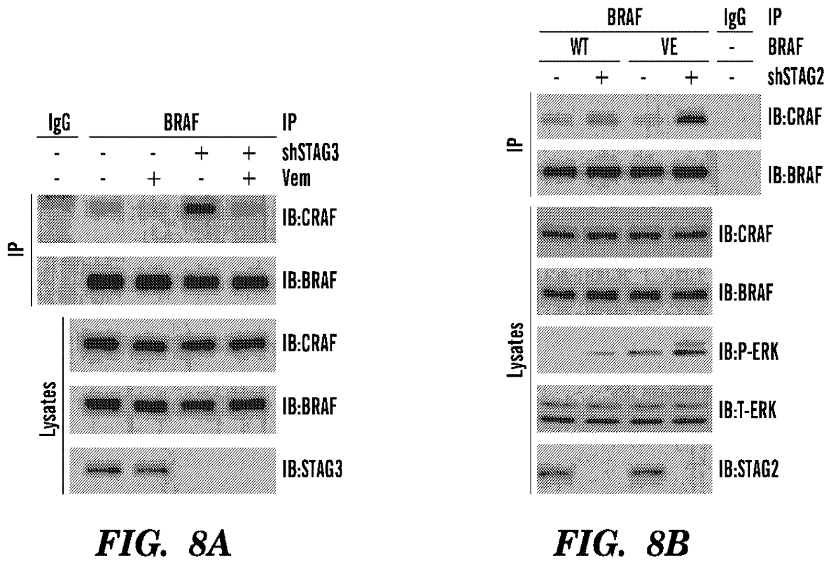

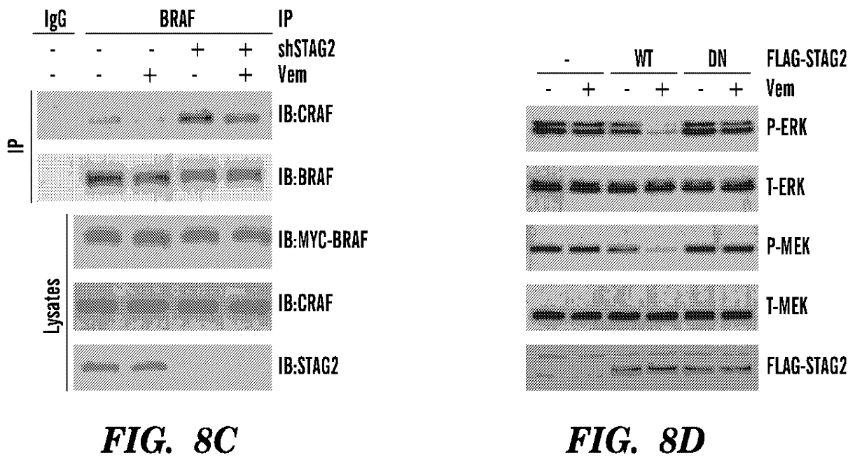

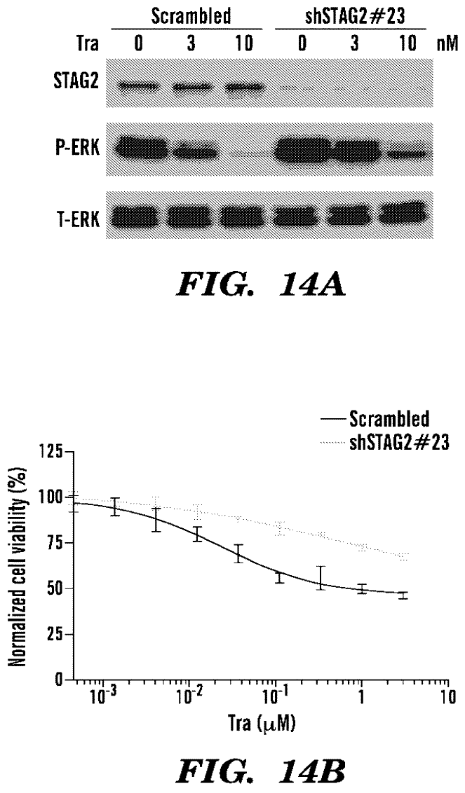

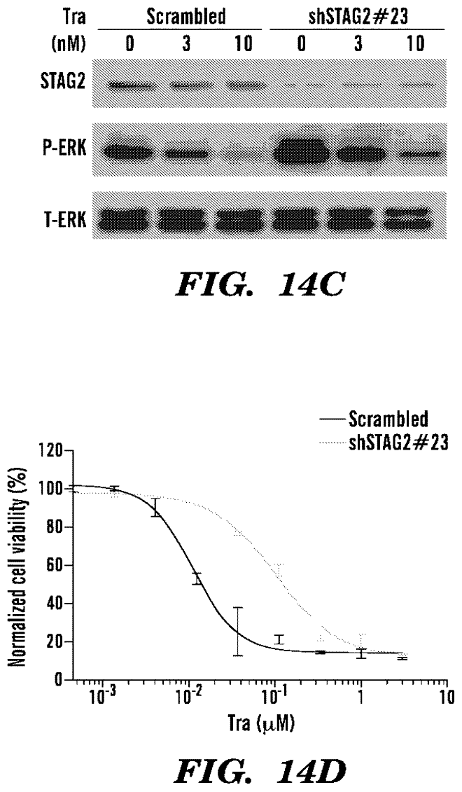

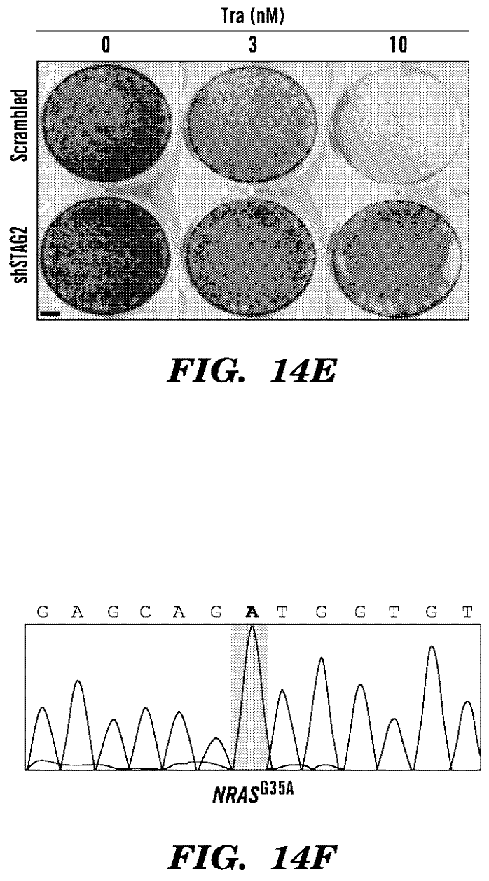

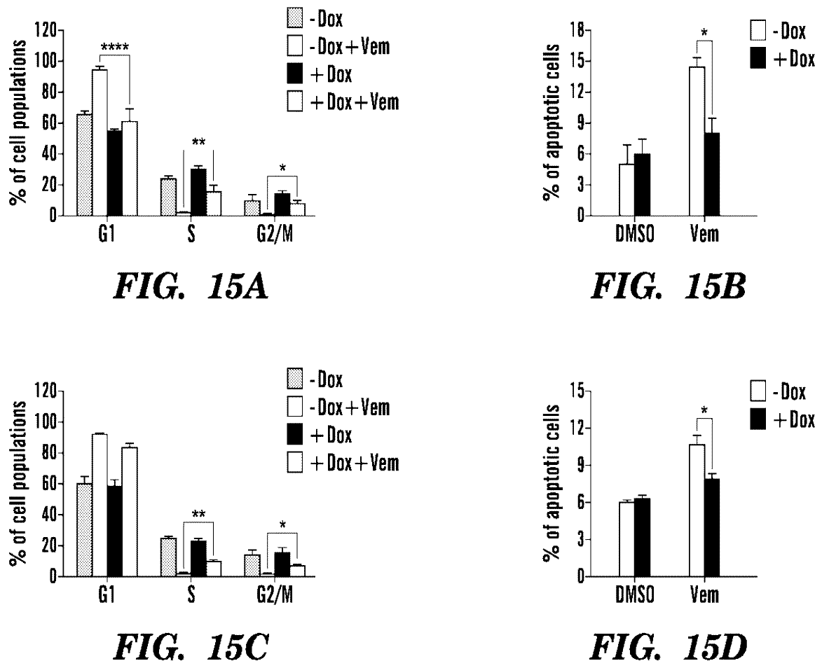

To examine whether loss of Stag2 or Stag3 is sufficient to confer resistance to BRAF inhibition, at least two independent shRNAs were used to knock down expression of either STAG2 or STAG3 in various BRAF-mutant melanoma cell lines and examined whether this altered their sensitivities to pharmacological inhibition of BRAF. In cell viability assays using the tetrazolium dye MTS, A375 cells expressing STAG2-specific shRNA showed lower sensitivity to the BRAFi dabrafenib, as compared to cells expressing a scrambled control shRNA (FIG. 2A). Knockdown of STAG2 also resulted in increases of basal levels of phosphorylated (p)-ERK and in a reduction in the ability of dabrafenib to inhibit ERK phosphorylation in these cells (FIG. 2B). However, levels of phosphorylated AKT (p-AKT) and ribosomal protein S6 (p-S6) were not affected by knockdown of STAG2 (FIG. 2B). Similarly, inducible expression of an independent shRNA targeting STAG2 expression also decreased the sensitivity of SKMEL28, A375 and M14 cells to either dabrafenib or vemurafenib (FIGS. 2C, 2D, 7A-7H). Knockdown of STAG2 also decreased sensitivity of A375 cells to the MEKi trametinib either alone or in combination with dabrafenib (FIGS. 7N-7Q). In addition to BRAF mutant melanoma cells, it was found that in NRAS-mutant SKMEL30, SKMEL103 and 501MEL melanoma cells, depletion of STAG2 by shRNA treatment also induced resistance to trametinib, as indicated by its inability to inhibit ERK phosphorylation and reduce cell viability in these cells (FIGS. 2E & 2F and FIGS. 14A-14E). Similar to STAG2, knockdown of STAG3 in BRAF-mutant melanoma cells also resulted in decreased sensitivity to dabrafenib or vemurafenib with regard to cell viability and ERK inhibition (FIGS. 2G, 2H, and 7I-7L). Co-depletion of both STAG2 and STAG3 further reduced the ability of vemurafenib to inhibit p-ERK signaling in A375 cells, as compared to that observed with knockdown of STAG2 or STAG3 alone (FIG. 7.). Furthermore, it was found that either STAG2 or STAG3 knockdown in A375 cells markedly impaired the changes in cell cycle progression and reduced the percentages of annexin V+apoptotic cells in response to vemurafenib treatment (FIGS. 15A-15D). These data indicate that loss of STAG2 or STAG3 decreases sensitivity to BRAF pathway inhibition through reactivation of ERK signaling.

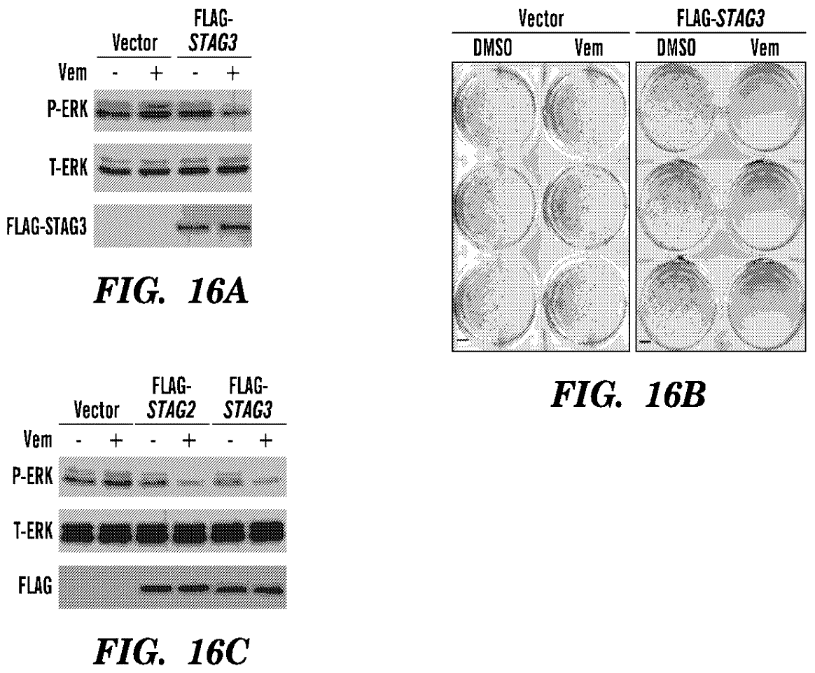

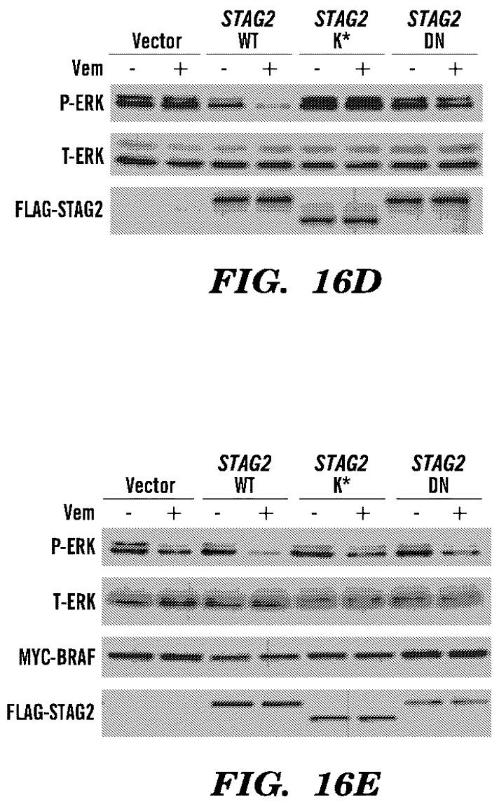

Next, the effects of ectopic expression of STAG2 and STAG3 were examined with respect to BRAFi sensitivity in BRAF-mutant melanoma cells. Expression of Flag-tagged wild-type STAG2 and STAG3, but not STAG2^{Lys1083*} or STAG2^{Asp193Asn}, in WM902-BR cells increased the ability of vemurafenib to inhibit ERK activity and to reduce colony formation in soft agar assays (FIGS. 7N-7O). Similar effects of ectopic expression of STAG2 or STAG3 on vemurafenib-induced ERK inhibition were also observed in HEK293 cells that co-express BRAF^{Val600Glu} and in WM983-BR. M14, and LOX-IVMI cells (FIGS. 16A-16E). These results further indicate that STAG2 and STAG3 regulate the sensitivity of melanoma cells to BRAFi.

HEK293 cells were transfected with MYC-tagged BRAF Val600Glu together with FLAG-tagged wild-type STAG3 (WT). Pro272Ser (PS) or Arg508Gln (RQ) mutants. Cells were treated with 10 μM vemurafenib for 2 h (data not shown). Cell lysates were used for Western blotting with indicated antibodies. Experiment was performed 3 times. In addition. M14 cells stably expressing FLAG-tagged wild-type STAG2. STAG3 or control vector were treated with 30 nM vemurafenib for 2 h. Cell lysates were used for Western blotting with indicated antibodies. Experiment was performed 3 times (data not shown).

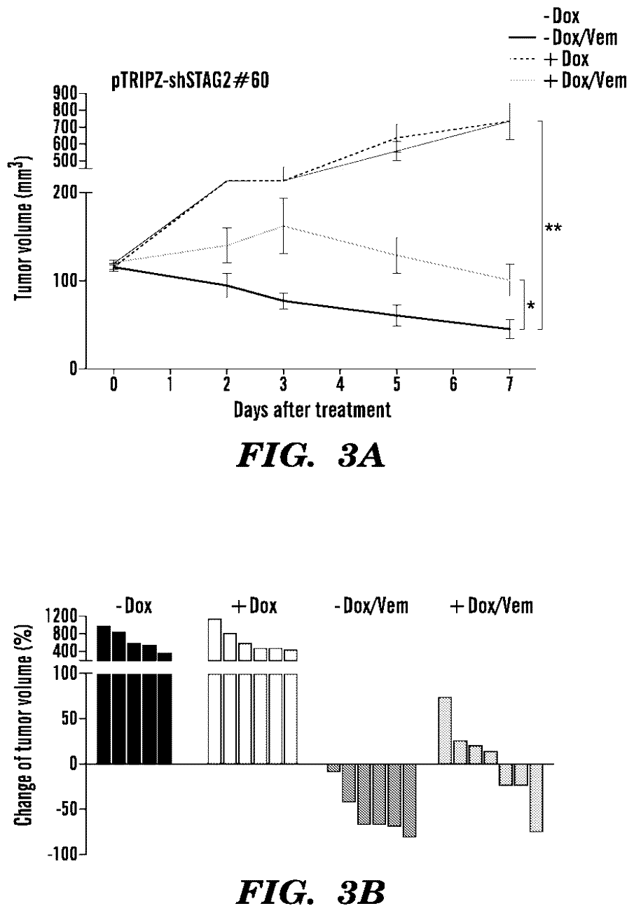

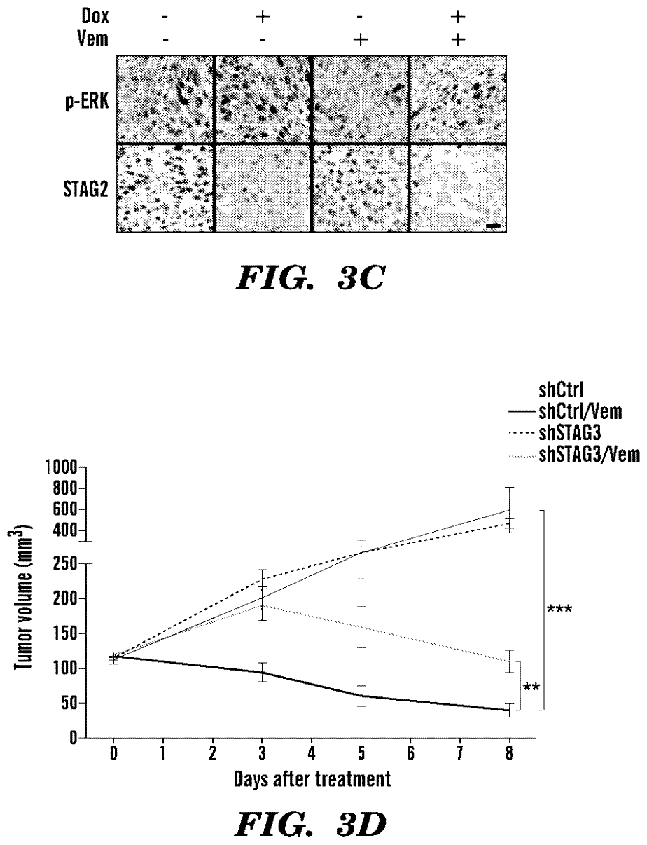

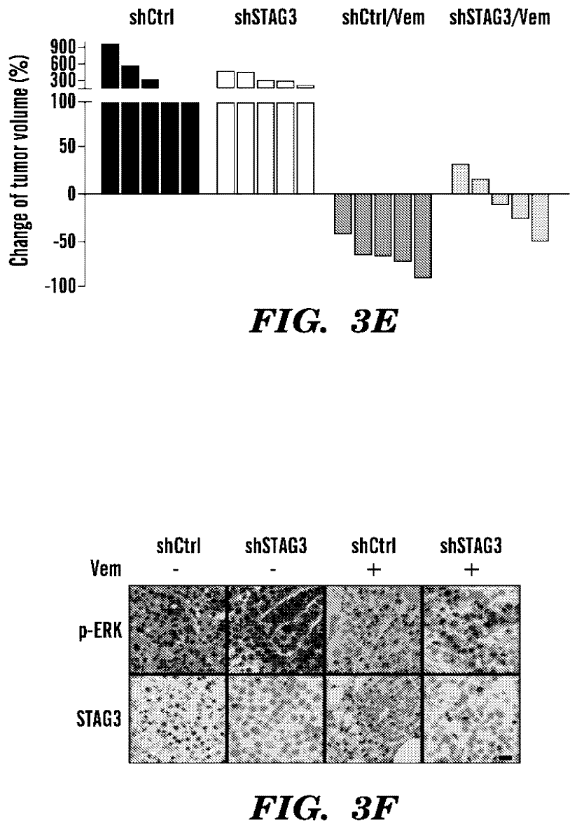

It was then sought to determine whether Stag2 and Stag3 regulate responses to BRAF inhibition in melanoma in vivo. A375 cells that inducibly expressed theSTAG2-specific shRNA were grown as xenograft tumors in nude mice to assess their sensitivities to vemurafenib. Silencing of Stag2 did not significantly affect A375 xenograft tumor growth in nude mice (FIG. 3A). However, tumors with STAG2 knockdown showed significantly decreased sensitivity to vemurafenib-induced tumor shrinkage as compared to that in control mice (FIGS. 3A, 3B). Immunohistochemical analysis revealed that pERK levels in STAG2-knockdown tumors treated with vemurafenib were higher than those in the tumors of the control group (FIG. 3C). Similar effects to the responses of A375 xenograft tumors to vemurafenib were observed for knockdown of STAG3 (FIGS. 3D-3F). Together these data indicate that loss of STAG2 or STAG3 decreases the sensitivity of melanoma tumors to BRAF inhibition in vivo.

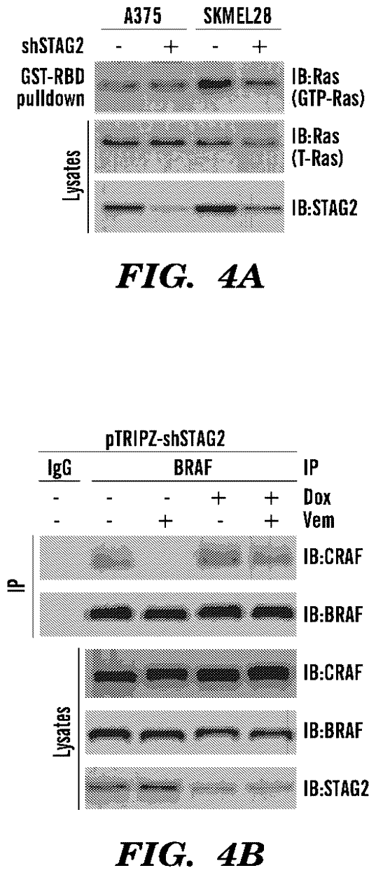

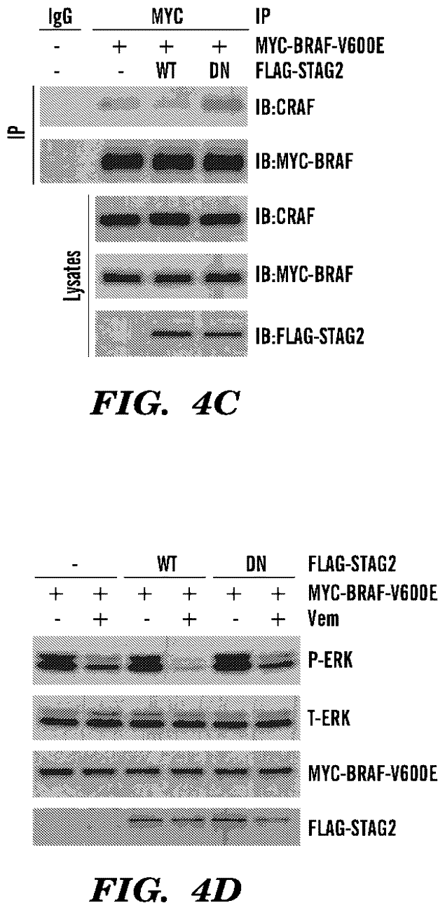

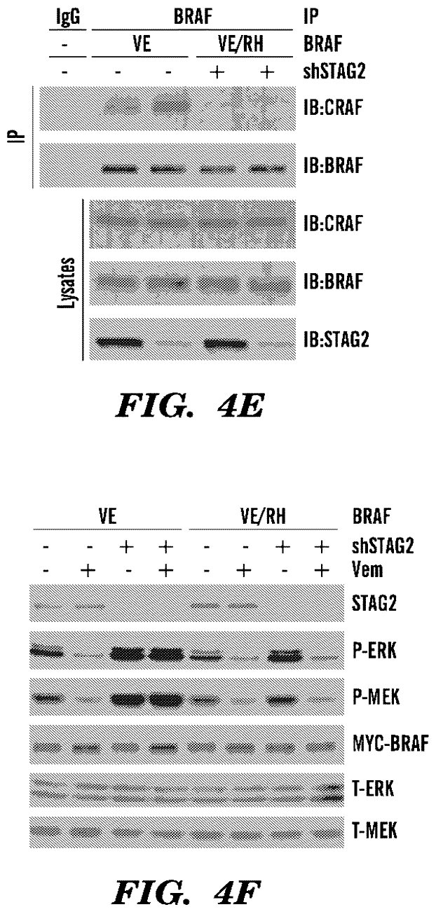

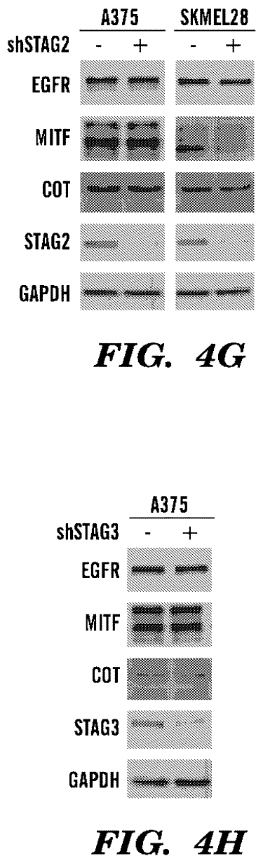

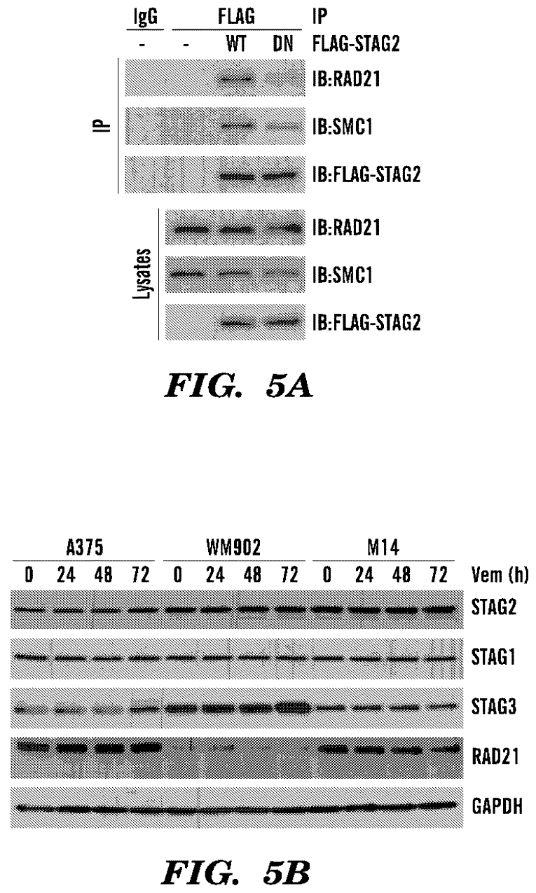

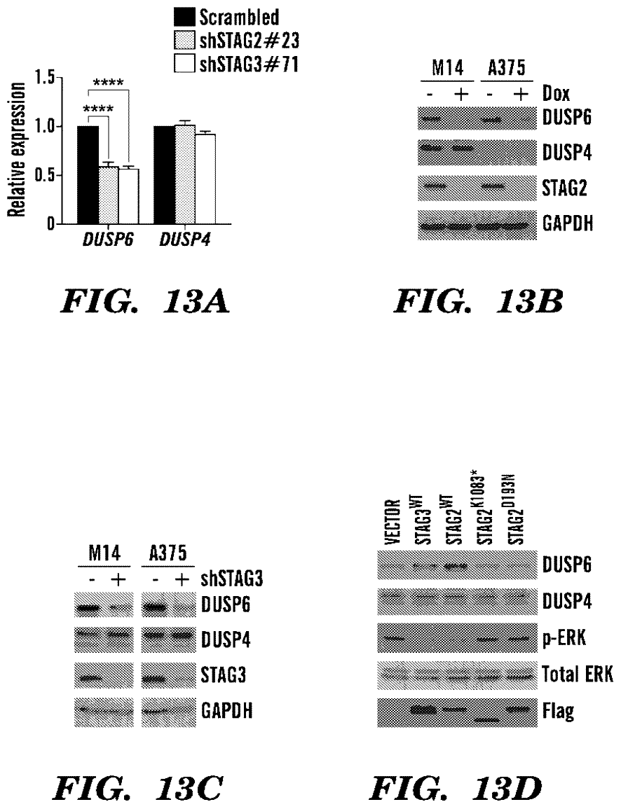

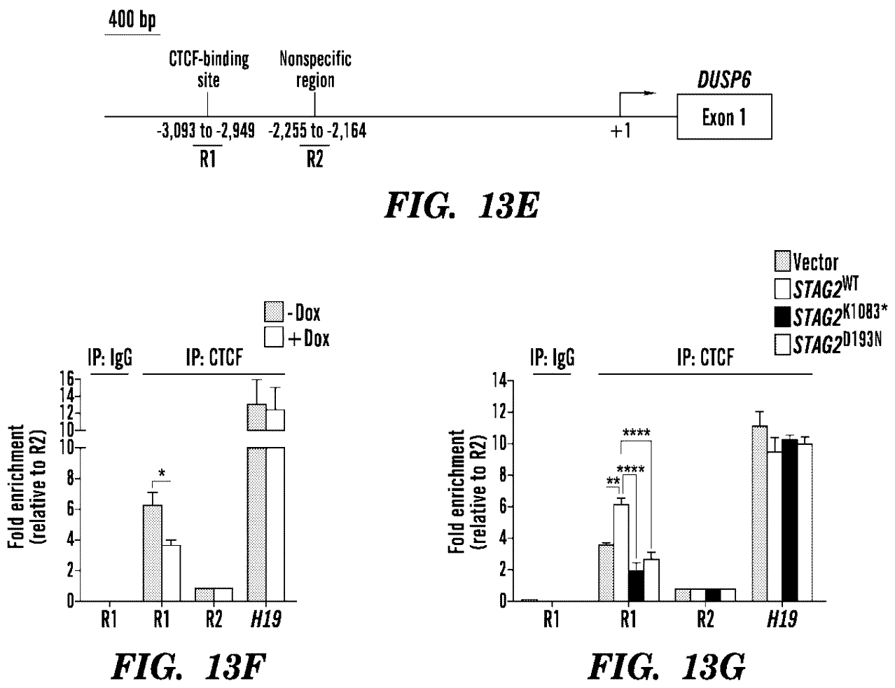

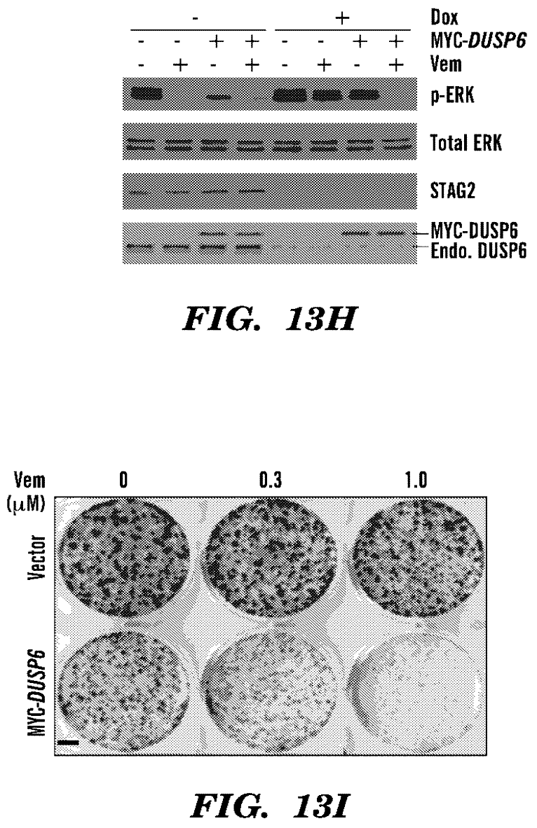

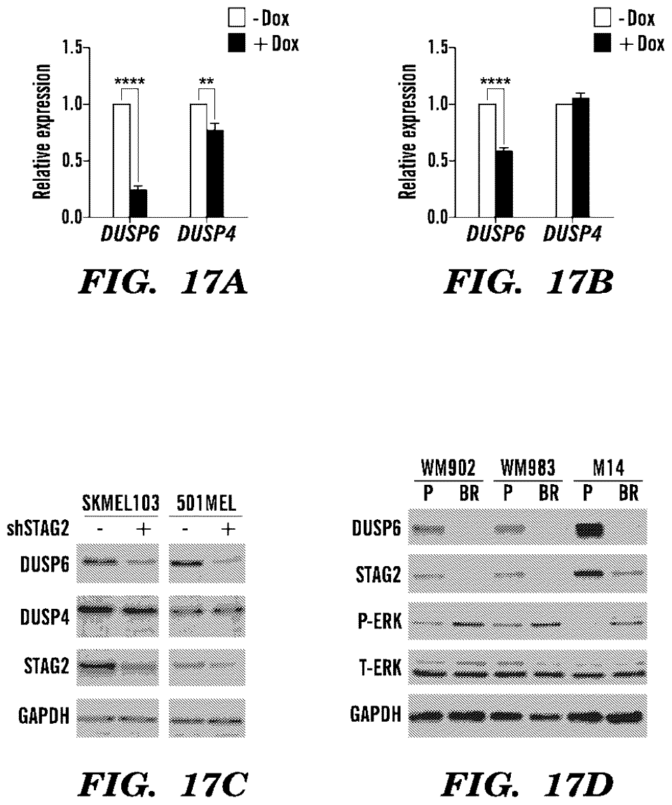

The molecular mechanism underlying the regulation of protein kinase signaling cascade RAF-MEK-ERK by STAG2 was investigated. The effect of STAG2 knockdown on RAS GTPase activation in melanoma cells was examined. Knockdown of STAG2 did not affect the levels of GTP-bound Ras in A375 or SKMEL28 cells, as demonstrated in RAF1 RAS-binding domain (RBD) pull-down assays using glutathione-S-transferase (FIG. 4A). Because silencing of STAG2 caused significant increases in the basal levels of p-ERK (FIG. 2), it was next assessed whether STAG2 regulates ERK activities through ERK phosphatases, such as DUSP4 and DUSP6, which are key players in the BRAF-MEK-ERK pathway⁶. shRNA-mediated knockdown of STAG2 or STAG3 expression led to significant decreases of DUSP6 but not DUSP4 mRNA levels in A375 and M14 melanoma cells (FIGS. 13A & FIGS. 17A, 17B). Similar effects for DUSP6 protein levels were observed in melanoma cells with STAG2 or STAG3 knockdown (FIGS. 13A, 13C, & 17C). In addition, the effect of ectopic STAG2 expression on DUSP6 protein abundance was assessed. Expression of wild-type STAG2 but not STAG2^{Lys1083}* or STAG2^{Asp193Asn}, increased DUSP6 protein expression in HEK293 cells (FIG. 13D). DUSP6 protein expression was also reduced in BRAFi-resistant melanoma cell lines, as compared to their parental BRAFi-sensitive counterparts (FIG. 17D). These findings indicate that STAG2 controls the expression of DUSP6 in melanoma cells.

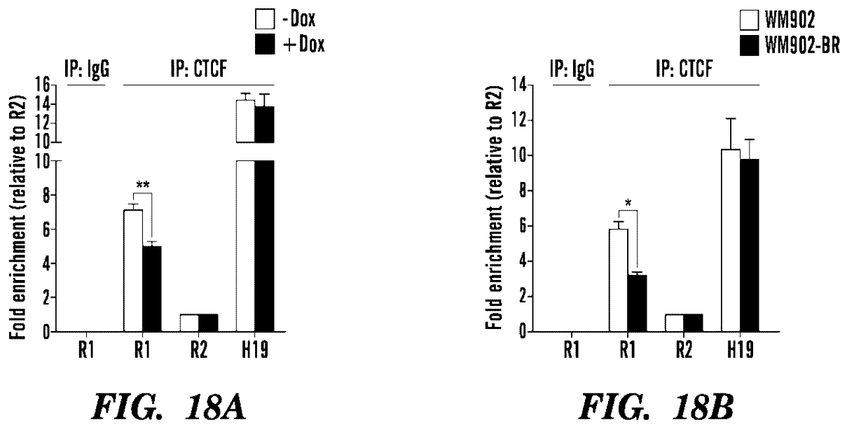

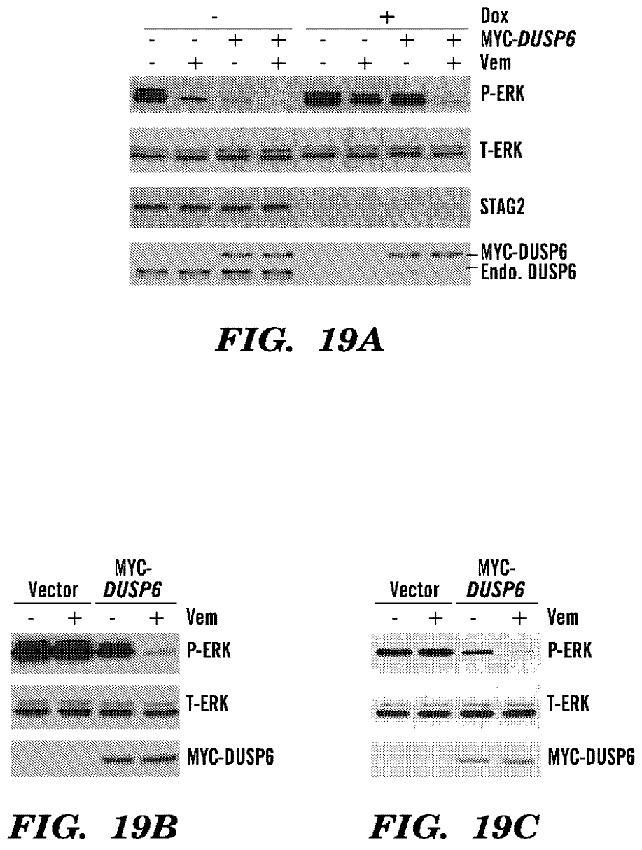

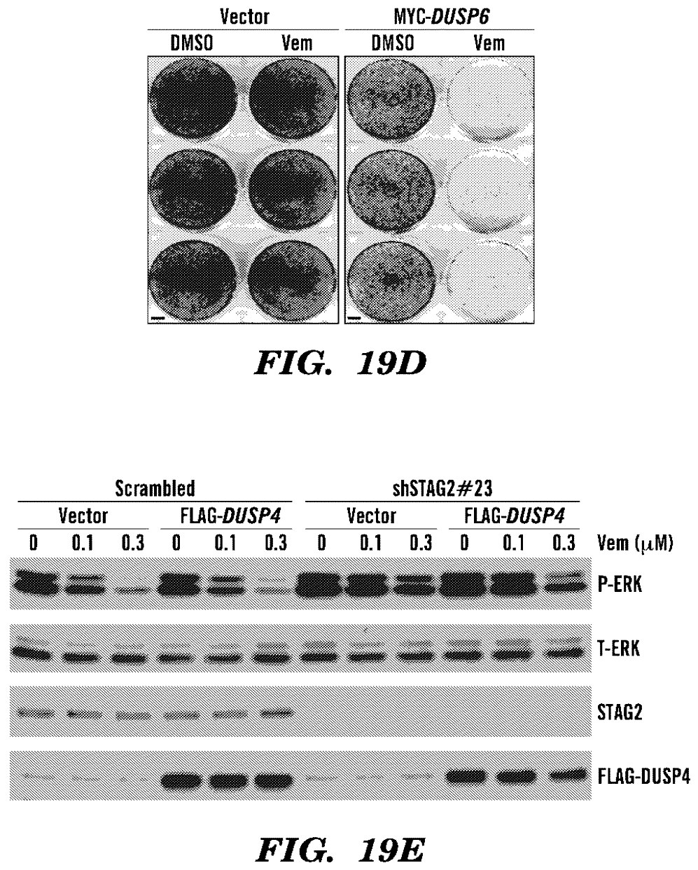

The cohesin complex, of which STAG2 is a major component, can interact with CCCTC-binding factor (CTCF) and participate in DNA-looping interactions between promoters and distal regulatory DNA elements, thereby controlling gene expression^{12,27,28}. The promoter region of DUSP6 contains a CTCF-binding site (FIG. 13E), as identified in previous whole-genome chromatin immunoprecipitation followed by sequencing (ChIP-seq) analyses of CTCF-binding sites²⁹. ChIP analyses were performed in A375 and M14 melanoma cells with a CTCF-specific antibody, thus confirming that CTCF binds to the DUSP6 locus in these cells (FIG. 13F, 18A). shRNA-mediated knockdown of STAG2 expression significantly reduced the binding of CTCF-binding site in the H19 locus (FIG. 13F, 18A). Expression of STAG2^{Lys1083}* or STAG2^{Asp193Asn} abolished the binding of CTCF to the DUSP6 locus in LOX-IVMI cells, as compared to cells expressing Flag-tagged wild-type STAG2 (FIG. 13G). Similarly, it was found that binding of CTCF to the DUSP6 locus is much stronger than WM902 cells than in WM902 BR cells that carry the STAG2^{Lys1083}* mutation (FIG. 18B). Finally, to determine whether DUSP6 mediates the effect of STAG2 on the BRAFi response. Myc-tagged DUSP6 and the STAG2-specific shRNA were overexpressed in A375 cells; restoration of DUSP6 expression attenuated the induction of basal p-ERK levels by STAG2 silencing and enhanced the ability of vemurafenib to inhibit ERK activities and to reduce clonogenic growth in cells after STAG2 knockdown (FIGS. 13H, 13I). Similar effects of ectopic DUSP6 expression were also observed in M14, WM902-BR and WM983-BR melanoma cells (FIG. 19E). Taken together, these results indicate that loss of STAG2 inhibits CTCF-mediated expression of DUSP6, leading to reactivation of MEK-ERK signaling in BRAFi-treated melanoma cells.

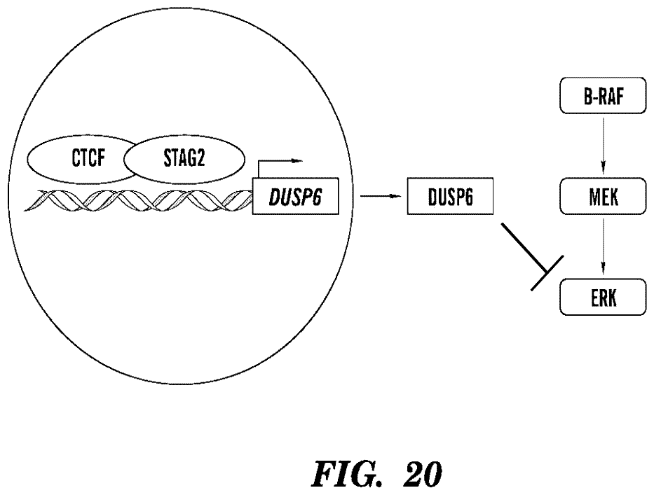

These findings not only reinforce the concept that reactivation of ERK signaling represents a major resistance mechanism of BRAF pathway inhibition^{3,6,9}, but they also reveal a previously unappreciated connection between STAG proteins and ERK signaling. With the recent advances in the field of cancer genomics, the genes encoding components of the cohesin complex have emerged as frequent targets of somatic alterations in a wide variety of cancers of different origins^{11,12}. In addition to a canonical function in sister chromatid cohesin and segregation, the cohesin complex has a notable role in chromatin organization and transcription^{11,12}. Whereas SMC1, SMC3 and RAD21 form the cohesin ring structure that entraps sister chromatids, STAG2 interacts with RAD21 at the base of the ring and has a regulatory rather than a structural role in the cohesin complex. How STAG2 exerts its tumor suppressor functions remains an open question. Sister chromatid cohesin, instead of regulation of the global transcription program, was proposed as the major tumor suppressor function of STAG2¹³. Inactivation of STAG2 causes cohesin defects and aneuploidy in glioblastoma and colorectal carcinoma cell lines¹³. However, cytogenetic abnormalities do not appear to be associated with STAG2 mutations in leukemia, bladder cancer, and Ewing sarcoma according to several recent cancer genomics studies^{14,18,19}, suggesting that aneuploidy may not underlie the tumor suppression role of STAG2 in these cancers. Notably, CTCF- and cohesin-binding sites have been recently reported to be frequently mutated in various types of cancers³⁰. The discovery of the regulation of the ERK signaling pathway by STAG2 or STAG3 not only supports a critical role of STAG2 in regulating DUSP6 gene expression through CTCF (FIG. 20), but it also reveals a new dimension of their tumor suppressive capacity.

### Methods

Patient samples and immunohistochemistry (IHC): Patients with metastatic melanoma containing BRAF^{Val600} mutations (confirmed by genotyping) were enrolled in clinical trials testing treatment with a BRAF inhibitor alone or in combination with a MEK inhibitor. Patients gave consent for tissue acquisition as per the Institutional Review Board (IRB)-approved protocol. The clinical trial numbers include: NCT01006980, NCT01107418, NCT01264380, NCT01248936, NCT00949702, and NCT01072175. Tumor biopsies were performed before treatment and at the time of progression. Formalin-fixed tissue was analyzed to confirm that viable tumor was present, using hematoxylin and eosin (H&E) staining. No statistical method was used to predetermine sample size for the IHC analysis. No samples were excluded from the IHC analysis. The investigators were blinded to group allocation and outcome assessment.

Tumor biopsies were sectioned at 4 μm and stained manually with primary antibodies for STAG2 (1:100, Cell Signaling, SC-81852) and STAG3 (1:200, Abcam, Ab185109) followed by a secondary horseradish-peroxidase-conjugated antibody (DAKO K4003 for STAG3 or DAKO K4001 for STAG2) and BAJORAN™ Purple chromogen kit (Biocare Medical™ BJP811). All slides were counterstained with hematoxylin (Vector H-3401). Stained slides were interpreted by a dedicated dermatopathologist.

Sequencing: Genomic DNA samples extracted from pre-treatment and post-relapse fresh-frozen paraffin-embedded (FFPE) tissues of a patient who relapsed from vemurafenib treatment were subjected for whole-exome sequencing analysis using Agilent SURESELECT™ Human All Exon 51M kit at BGI (Beijing, China). Reads were mapped to hg19 using bwa³¹. PCR duplicates and non-uniquely mapped reads were discarded using samtools³². VarScan2³³ was further used to call somatic mutations and results were annotated by Annovar³⁴. Mutations that mapped to segmental duplications or were annotated in 1000 Genome Project and dbsnp138 were filtered afterwards. Only non-synonymous, stop-gain, stop-loss mutations were selected for later analysis. High confident mutations were further picked based on the total coverage, coverage for reference allele, coverage for altered allele and functional prediction from Polyphen2³⁵. For high throughput Sanger sequencing, all coding exons and intron-exon junctions in the STAG2 and STAG3 genes were amplified by PCR, followed by DNA sequencing and single nucleotide polymorphism (SNP) discovery data analysis at Polymorphic DNA Technologies™ (Alameda, CA). To confirm the STAG2 mutations found in patient samples and cell lines, PCR reactions were performed for Exon 7 and Exon 29 with the following pairs of primers: 7F, 5′-GATAGTGGAGATTATCCACTTAC-3′ (SEQ ID NO. 5), 7R, 5′-CTGCCAGGGTGCTTGTATGTCG-3′ (SEQ ID NO. 6): 29F, 5′-ATGCCTATGCTCGCACAACT-3′ (SEQ ID NO. 7), 29R, 5′-ATACTGAGTCCATTTCCCTATGC-3′ (SEQ ID NO. 8). NRAS^{Gly12Asp} mutation in 501MEL cells³⁶ was confirmed by PCR amplification of exon 2 with primers: 2F, GAACCAAATGGAAGGTCACA (SEQ ID NO. 9) and 2R, TGGGTAAAGATGATCCGACA (SEQ ID NO. 10), followed by Sanger sequencing.

Materials: Information on the antibodies used in this study are listed in the following Table:

| | | | | Technique |
|---|---|---|---|---|
| Antigen | Manufacturer | Clone | Catalog # | and dilution |
| phospho-ERK | Cell Signaling | N/A | 9101 | IHC (1:400); |
| (Thr202/ | Technology | | | WB (1:3000) |
| Tyr204) | | | | |
| ERK | Cell Signaling | 137F5 | 4695 | WB (1:3000) |
| | Technology | | | |
| MYC | Cell Signaling | 9B11 | 2276 | WB (1:1000) |
| | Technology | | | |
| phospho-AKT | Cell Signaling | C31E5E | 2965 | WB (1:1000) |
| (Thr308) | Technology | | | |
| AKT | Cell Signaling | C67E7 | 4691 | WB (1:1000) |
| | Technology | | | |
| phospho-S6 | Cell Signaling | N/A | 2215 | WB (1:3000) |
| (Ser240/ | Technology | | | |
| Ser244) | | | | |
| S6 | Cell Signaling | 5G10 | 2217 | WB (1:3000) |
| | Technology | | | |
| SMC1 | Cell Signaling | 8E6 | 6892 | WB (1:1000) |
| | Technology | | | |
| EGFR | Cell Signaling | N/A | 2232 | WB (1:1000) |
| | Technology | | | |
| GAPDH | Cell Signaling | 14C10 | 2118 | WB (1:5000) |
| | Technology | | | |
| STAG2 | Santa Cruz | N/A | SC-81852 | IHC (1:100); |
| | | | | WB (1:1000) |
| STAG3 | Abcam | N/A | ab185109 | IHC (1:200); |
| | | | | WB (1:1000) |
| DUSP4 | Abcam | N/A | ab72593 | WB (1:1000) |
| DUSP6 | Abcam | N/A | ab76310 | WB (1:1000) |
| RAD21 | Abcam | N/A | ab992 | WB (1:1000) |
| FLAG | Sigma | M2 | F3165 | IHC (1:500); |
| | | | | WB (1:1000) |
| pan-Ras | ThermoScientific | N/A | 16117 | WB (1:1000) |
| MITF | ThermoScientific | N/A | MS-771-P1 | WB (1:200) |
| STAG1 | Novus Biologics | N/A | NB100-298 | WB (1:1000) |
| COT | Biorbyt | N/A | orb127540 | WB (1:250) |
| CTCF | Diagenode | N/A | C1541210 | WB (1:500) |

Vemurafenib, dabrafenib, and trametinib were purchased from Selleck Chemicals. Doxycycline, crystal violet, and iodonitrotetrazolium chloride were purchased from Sigma. pLEX-HA-DUSP6-MYC was provided by Dr. Igor Astsaturov and pLJM1-STAG2 was provided by Dr. Todd Waldman through Addgene. pLX304-DUSP4-V5 was purchased from the DNASU Plasmid Repository. The Flag-tag-encoding sequence was added to the N-terminus-encoding sequence of DUSP4 to generate pLX304-FLAG-DUSP4-V5, using PCR-based methods. pBabe-FLAG-STAG2 was generated by PCR-based subcloning from pLJM1-STAG2. pBabe-MYC-BRAF construct was generated by PCR-based subcloning from pLHCX-FLAG-BRAF³⁷. pBabe-FLAG-STAG3 was generated by PCR-based subcloning using STAG3 cDNA purchased from GE Dharmacon as a template. Various mutated STAG2. STAG3 and BRAF alleles were generated using PCR mutagenesis and verified by sequencing. pLKO constructs containing shRNAs against human STAG2 (shSTAG2 #23:TRCN0000152523) and STAG3 (shSTAG3 #96:TRCN0000137596; shSTAG3 #71:TRCN0000138271:shSTAG3 #69:TRCN0000138869) were purchased from Sigma. pTRIPZ inducible lentiviral human STAG2 shRNA (shSTAG2 #60 CloneID:V2THS_12573) and STAG3 shRNA (shSTAG3 #55 CloneID:V3THS 301555) were purchased from GE Dharmacon.

Cell culture: All melanoma cell lines used in this study contain BRAF^{Val600Glu} mutations, except as otherwise indicated. A375 and SKMEL28 cells were purchased from the American Type Culture Collection (ATCC). LOX-IVMI cells were obtained from the Division of Cancer Treatment and Diagnosis. National Cancer Institute. (NCI-DCTD) repository. WM902, WM902-BR, WM983, WM983-BR and MEL1617 cell lines were obtained from Dr. Meenhard Herlyn (Wistar Institute). Immortalized Braf-null mouse embryonic fibroblasts (MEFs) were a gift from Dr. Catrin Pritchard (University of Leicester)³⁸. 501MEL and SKMEL103 cells, harboring NRAS mutations, were gifts from Dr. Lynda Chin (MD Anderson Cancer Center) and Dr. Jonathon Zippin (Weill Cornell Medical College), respectively. These cell lines were not authenticated by the inventors. WM902, WM983, M14, MEL1617, SKMEL28, A375, LOX-IVMI, 501MEL and SKMEL103 cells were cultured in RPMI containing 10% FBS (FBS) and penicillin-streptomycin-glutamine (PSG). HEK293 and MEF cells were maintained in Dulbecco's modified Eagle's medium (DMEM) containing 10% FBS and PSG. WM902-BR, WM983-BR, M14-BR, A375-BR and Mel1617-BR cells were maintained in complete medium supplemented with vemurafenib or dabrafenib. A375-BMR and MEL1617-BMR cells were maintained in complete medium supplemented with dabrafenib and trametinib. For the cell-viability analysis, cells were seeded in 96-well plates, and drug treatment was started the following day. After a 72-h incubation, the MTS assay was performed according to the manufacturer's instructions (Promega). All cell lines tested negative for mycoplasma, using the MYCOSENSOR™ PCR Assay Kit (Agilent Technologies). Transfection, retroviral infection and lentiviral infection were performed as previously described²⁵. When indicated, stable populations were obtained and maintained by selection with puromycin (Sigma). Clonogenic growth²⁵ and anchorage-independent growth soft-agar assays³⁹ were performed as previously described.

Cell cycle and apoptosis analyses. For cell cycle analysis, cells were fixed drop wise with 70% ice-cold ethanol for 30 min on ice and suspended in PBS containing 10 μg/ml of propidium iodide (PI) and 10 μg/ml of RNase A. PI-stained samples were analyzed for cell cycle progression by flow cytometry, using a FACSCALIBUR (Becton and Dickenson) apparatus, followed by data analysis using the FlowJo software (TreeStar). For apoptosis analysis, apoptotic cells were detected using BD FITC Annexin V Apoptosis Detection Kit followed by flow cytometry analysis.

Western blotting and immunoprecipitation: Western blotting and immunoprecipitation were performed as previously described³⁹. Ras activity assay was performed using active Ras pull-down and detection kit according to manufacturer's instructions (Thermo Scientific). Briefly, 500 μg of cell lysates were incubated with GST-Raf1-RBD and glutathione resin at 4° C. for 1 hr. After washing, the active Ras was eluted by 2× reducing sample buffer and subjected for SDS-PAGE and immunoblotting.

Animal studies: All studies and procedures involving animal subjects were performed following institutional IACUC guidelines. For xenograft models, 6-week-old female athymic mice (NCr^{nu/nu}) were purchased from Taconic farms. Animals were allowed a 1-week adaptation period after arrival. A375 cells (1×10⁶ in 0.2 ml of basal culture medium) were injected subcutaneously in the right lateral flank. To induce silencing of STAG2 in vivo, 2 mg/mL doxycycline and 5% sucrose was added to the drinking water 13 days post inoculation. Doxycycline-containing water was changed every three days. Vemurafenib diet (1.42 g/kg to achieve a 25 mg/kg daily dose) and control diet were prepared at Harlan Laboratories (Madison, WI). Animals were randomly assigned to 4 groups that were administered vehicle (5% sucrose in water), doxycycline, vehicle and vemurafenib, or both doxycycline and vemurafenib, by the Research Randomizer. The investigators were not blinded to group allocation or outcome assessment. No statistical method was used to predetermine sample size. Treatment began when the tumor volume reached between 80 to 120 mm³. Tumor dimensions were measured with calipers and volumes calculated using the following formula: (D×d²)/2, in which D represents the large diameter of the tumor, and d represents the small diameter of the tumor. Animals were euthanized at the end of the experiments or when the tumor size reached 1.5 cm in any dimension.

Mouse tissue sections were prepared for immunohistochemistry as previously described²⁵. Briefly, harvested mouse tissues were fixed in 10% neutral buffered formalin and embedded in paraffin. Slides were deparaffinized using HISTOCHOICE™ clearing reagent (Amresco) and rehydrated with water. Antigen retrieval for formalin fixed tissue sections was performed by heating slides in a pressure cooker for 10 min in citrate antigen retrieval solution. After wash with PBS, endogenous peroxidase activity was quenched with 3% hydrogen peroxide in PBS for 10 min at room temperature. For p-ERK. STAG2 and STAG3 staining, slides were blocked with 5% goat serum in PBS for 30 min and incubated with the primary antibodies for STAG2 (1:100, Santa Cruz, SC-81852), STAG3 (1:200, Abcam, Ab185109), p-ERK (1:400, Cell Signaling) at 4° C. overnight, followed by incubation with biotinylated anti-rabbit IgG for p-ERK and STAG3, and anti-mouse IgG for STAG2 for 30 min (Vector Laboratories). All slides were then incubated with avidin-biotin peroxidase (ABC) complex for 30 min and the signals visualized using DAB Substrate Kit (Vector Laboratories). The tissue sections were counterstained with Gill's hematoxylin QS and mounted with VECTAMOUNT™ after dehydration.

Reverse-transcription and real-time qPCR. RNA samples were isolated using the RNEASY™ Mini kit (Qiagen) and reverse-transcribed (˜2 μg) using the REVERTAID™ Reverse Transcription Kit (Thermo Fisher Scientific). qPCRs were performed using the SYBR GREEN I MASTER™ (Roche) on the LIGHT CYCLER 480™ Real-Time PCR instrument (Roche). Each sample was tested in triplicate, and the results were normalized to the expression of the housekeeping GAPDH gene. Specific primer sequences used in this study were as follows: DUSP4 forward, 5′-GGCTACATCCTAGGTTCGGT-3′ (SEQ ID NO. 11), DUSP4 reverse, 5′-CAGGATCTGCTCCAGGCT-3′ (SEQ ID NO. 12): DUSP6 forward, 5′-CTGCATTGCGAGACCAATCTA-3′ (SEQ ID NO. 13), DUSP6 reverse, 5′-CATCCGAGTCTGTT GCACTATT-3′ (SEQ ID NO. 14): GAPDH forward, 5′-ATCACTGCCACCCAGAAGAC-3′ (SEQ ID NO. 15), GAPDH reverse, 5′-CAGTGAGCTTCCCGTTCAG-3′ (SEQ ID NO. 16).

Chromatin immunoprecipitation. ChIP experiments were performed using the IDEAL™ ChIP-seq Kit for Transcription Factors (Diagenode) according to the manufacturer's instructions. Briefly, cells were grown to 80-90% confluency and then fixed with 1% formaldehyde solution (Sigma). Eight million cells were used per IP. Chromatin was sonicated into 200- to 800-bp fragments, and 1% of the chromatin was used to purify the input DNA fragments. Chromatin was immunoprecipitated with a CTCF-specific antibody or nonspecific rabbit IgG. qRT-PCR, using SYBR GREEN™, was performed to detect enriched DNA. Primers used for qPCR were as follows: CTCF R1 forward, 5′-CTGAAGACTGTCCGAAATTATGC-3′ (SEQ ID NO. 17): CTCF R1 reverse, 5′-CTGATTTCTCCCTACTGGTCAC-3′ (SEQ ID NO. 18): CTCF R2 forward, 5′-CTCCAACAGGTTTGCTCTTCT-3′ (SEQ ID NO. 19): CTCF R2 reverse, 5′-CCCGAGACGTTTCAGTCATT-3′ (SEQ ID NO. 20): H19 forward, 5′-CTGGTCTGTGCTGGCCACGG-3′ (SEQ ID NO. 21): H19 reverse, 5′-GCACCTTGGCTGGGGCTCTG-3′ (SEQ ID NO. 22).

### REFERENCES

1. Ribas, A. & Flaherty, K. T. BRAF targeted therapy changes the treatment paradigm in melanoma. Nat Rev Clin Oncol 8, 426-433 (2011).
2. Holderfield, M., Deuker, M. M., McCormick, F. & McMahon, M. Targeting RAF kinases for cancer therapy: BRAF-mutated melanoma and beyond. Nat Rev Cancer 14, 455-467 (2014).
3. Moriceau, G. et al. Tunable-combinatorial mechanisms of acquired resistance limit the efficacy of BRAF/MEK cotargeting but result in melanoma drug addiction. Cancer Cell 27, 240-256 (2015).
4. Van Allen, E. M. et al. The Genetic Landscape of Clinical Resistance to RAF Inhibition in Metastatic Melanoma. Cancer Discovery 4, 94-109 (2014).
5. Shi, H. et al. Acquired Resistance and Clonal Evolution in Melanoma during BRAF Inhibitor Therapy. Cancer Discovery 4, 80-93 (2014).
6. Lito, P., Rosen, N. & Solit, D. B. Tumor adaptation and resistance to RAF inhibitors. Nature Publishing Group 19, 1401-1409 (2013).
7. Kwong, L. N. et al. Co-clinical assessment identifies patterns of BRAF inhibitor resistance in melanoma. J Clin Invest 125, 1459-1470 (2015).
8. Wagle, N. et al. MAP kinase pathway alterations in BRAF-mutant melanoma patients with acquired resistance to combined RAF/MEK inhibition. Cancer Discovery 4, 61-68 (2014).
9. Long, G. V. et al. Increased MAPK reactivation in early resistance to dabrafenib/trametinib combination therapy of BRAF-mutant metastatic melanoma. Nature Communications 5, 5694 (2014).
10. Kandoth, C. et al. Mutational landscape and significance across 12 major cancer types. Nature 502, 333-339 (2013).
11. Solomon, D. A., Kim, J.-S. & Waldman, T. Cohesin gene mutations in tumorigenesis: from discovery to clinical significance. BMB Reports 47, 299-310 (2014).
12. Losada, A. Cohesin in cancer: chromosome segregation and beyond. Nat Rev Cancer 14, 389-393 (2014).
13. Solomon, D. A. et al. Mutational Inactivation of STAG2 Causes Aneuploidy in Human Cancer. Science 333, 1039-1043 (2011).
14. Balbás-Martinez, C. et al. Recurrent inactivation of STAG2 in bladder cancer is not associated with aneuploidy. Nat Genet 45, 1464-1469 (2013).
15. Guo, G. et al. Whole-genome and whole-exome sequencing of bladder cancer identifies frequent alterations in genes involved in sister chromatid cohesion and segregation. Nat Genet 45, 1459-1463 (2013).
16. Weinstein, J. N. et al. Comprehensive molecular characterization of urothelial bladder carcinoma. Nature 507, 315-322 (2014).
17. Solomon, D. A. et al. Frequent truncating mutations of STAG2 in bladder cancer. Nat Genet 45, 1428-1430 (2013).
18. Crompton, B. D. et al. The Genomic Landscape of Pediatric Ewing Sarcoma. Cancer Discovery 4, 1326-1341 (2014).
19. Tirode, F. et al. Genomic Landscape of Ewing Sarcoma Defines an Aggressive Subtype with Co-Association of STAG2 and TP53 Mutations. Cancer Discovery 4, 1342-1353 (2014).
20. Thol, F. et al. Mutations in the cohesin complex in acute myeloid leukemia: clinical and prognostic implications. Blood 123, 914-920 (2014).
21. Thota, S. et al. Genetic alterations of the cohesin complex genes in myeloid malignancies. Blood 124, 1790-1798 (2014).
22. Yoshida, K. et al. The landscape of somatic mutations in Down syndrome-related myeloid disorders. Nat Genet 45, 1293-1299 (2013).
23. Cancer Genome Atlas Research Network. Genomic and Epigenomic Landscapes of Adult De Novo Acute Myeloid Leukemia. New England Journal of Medicine 368, 2059-2074 (2013).
24. Hodis, E. et al. A Landscape of Driver Mutations in Melanoma. CELL 150, 251-263 (2012).
25. Yuan, P. et al. Phenformin enhances the therapeutic benefit of BRAFV600E inhibition in melanoma. Proc Natl Acad Sci USA 110, 18226-18231 (2013).
26. Villanueva, J. et al. Acquired Resistance to BRAF Inhibitors Mediated by a RAF Kinase Switch in Melanoma Can Be Overcome by Cotargeting MEK and IGF-1R/PI3K. Cancer Cell 18, 683-695 (2010).
27. Ong, C.-T. & Corces, V. G. CTCF: an architectural protein bridging genome topology and function. Nat. Rev. Genet. 15, 234-246 (2014).
28. Xiao, T., Wallace, J. & Felsenfeld, G. Specific sites in the C terminus of CTCF interact with the SA2 subunit of the cohesin complex and are required for cohesin-dependent insulation activity. Mol. Cell. Biol. 31, 2174-2183 (2011).
29. Ziebarth, J. D., Bhattacharya, A. & Cui, Y. CTCFBSDB 2.0: a database for CTCF-binding sites and genome organization. Nucleic Acids Res. 41, D188-D194 (2013).
30. Katainen, R. et al. CTCF-cohesin-binding sites are frequently mutated in cancer. Nat. Genet. 47, 818-821 (2015).
31. Li, H. & Durbin, R. Fast and accurate short-read alignment with Burrows-Wheeler transform. Bioinformatics 25, 1754-1760 (2009).
32. Li, H. et al. The sequence alignment-map format and SAMtools. Bioinformatics 25, 2078-2079 (2009).
33. Koboldt, D. C. et al. VarScan 2: somatic mutation and copy-number alteration discovery in cancer by exome sequencing. Genome Res. 22, 568-576 (2012).
34. Wang, K., Li. M. & Hakonarson, H. ANNOVAR: functional annotation of genetic variants from high-throughput sequencing data. Nucleic Acids Res. 38, e164 (2010).
35. Adzhubei, I. A. et al. A method and server for predicting damaging missense mutations. Nat. Methods 7, 248-249 (2010).
36. Lin, W. M. et al. Modeling genomic diversity and tumor dependency in malignant melanoma. Cancer Res. 68, 664-673 (2008).
37. Shen, C.-H. et al. Phosphorylation of BRAF by AMPK impairs BRAF-KSR1 association and cell proliferation. Mol. Cell 52, 161-172 (2013).
38. Kamata, T. et al. A critical function for BRAF at multiple stages of myelopoiesis. Blood 106, 833-840 (2005).
39. Zheng, B. et al. Oncogenic BRAF negatively regulates the tumor suppressor LKB1 to promote melanoma cell proliferation. Mol. Cell 33, 237-247 (2009).

### Example 2: Prediction of Responsiveness to Immune Checkpoint Blockade

Immune checkpoint inhibitors, such as anti-PD-1 and anti-PD-L1 antibodies, have recently shown significant clinical benefits in melanoma and other cancers. However, one of the main challenges of immune checkpoint blockade therapy is that the response rate is usually low. Thus, there is an unmet need to identify biomarkers that can predict which patients will benefit from PD-1/PD-L1 blockade therapy.

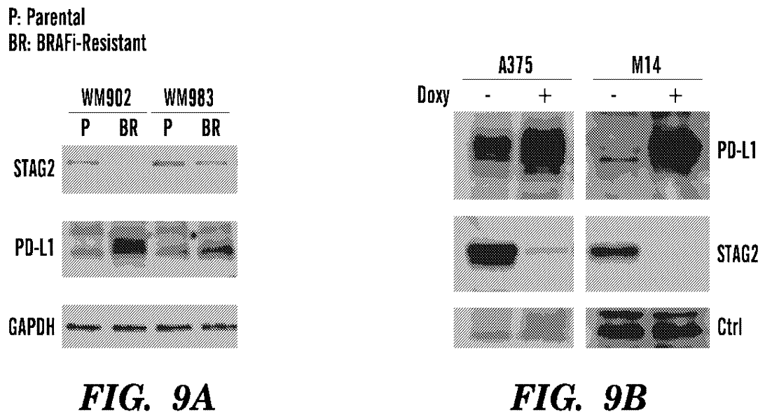

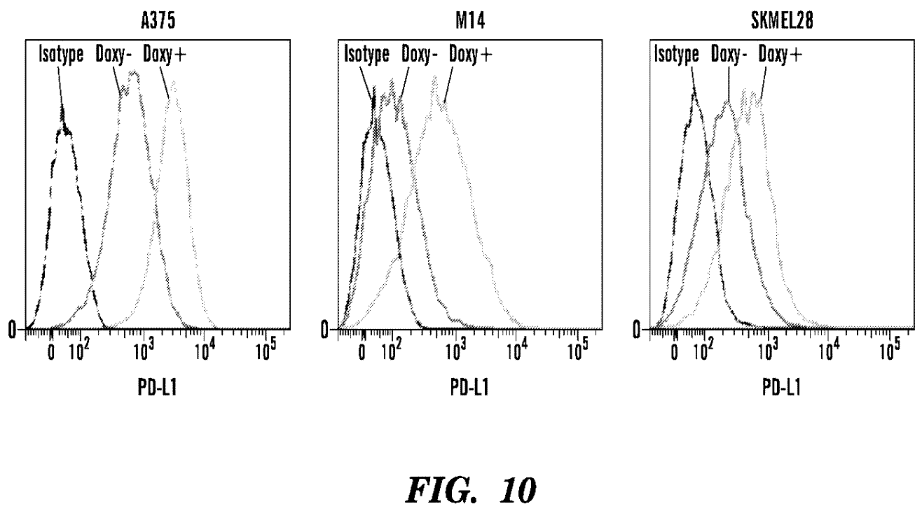

Provided herein are data showing that knockdown of Stag2 (stromal antigen 2) proteins in multiple melanoma cells led to increased total PD-L1 protein levels (FIGS. 9A-9B) and surface expression (FIG. 10).

Melanoma cell lines with Stag2 mutations also have higher levels of PD-L1 protein. Taken together, these results indicate that loss-of-function mutations in Stag2, as well as decreased expression of Stag2/3 proteins in cancer can serve as a predictive biomarker for sensitivity to PD-1 or PD-L1 checkpoint blockade therapy.

In addition to melanoma, it is also contemplated herein that the Stag2 mutations and/or Stag2/3 protein levels can be applied to subjects with other types of cancer, for example, cancers that have high frequencies of Stag2 mutation, such as urothelial bladder carcinomas, Ewing sarcoma, neuter myeloid leukemia, myelodysplastic syndrome, and acute megakaryoblastic leukemia. It is also contemplated herein that this technology can be used as a monitoring tool or can be used by oncologists at the point of care to determine if a particular patient should be assigned PD-1/PD-L1 therapy.

### Example 3: Prediction of Responsiveness to ERK Inhibition

Melanoma is the most dangerous form of skin cancer, with over 160,000 new diagnoses a year, causing over 75% of skin cancer deaths. Recent breakthroughs in skin cancer therapy have identified that a mutation in the BRAF gene occurs in over 50% of melanoma cases. New therapies have been developed that specifically inhibit the BRAF oncogene as a way of treating melanoma. However, some patients have an inherent resistance to BRAF inhibitors or may develop resistance to treatment with BRAF inhibitors. Such resistance to BRAF inhibitors is associated with a form of melanoma that is more aggressive and difficult to treat.

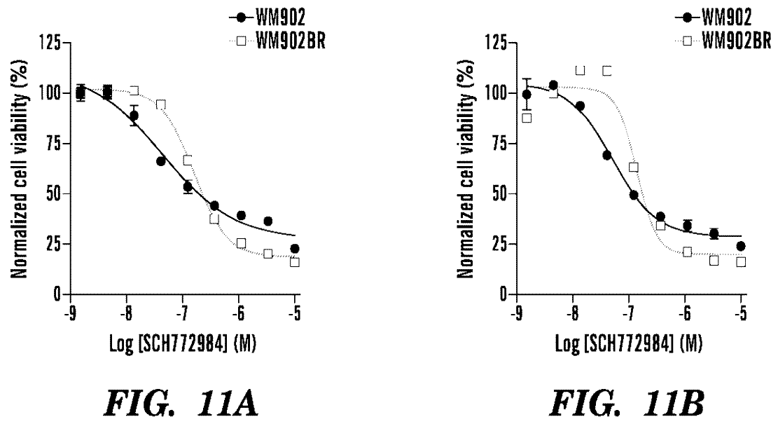

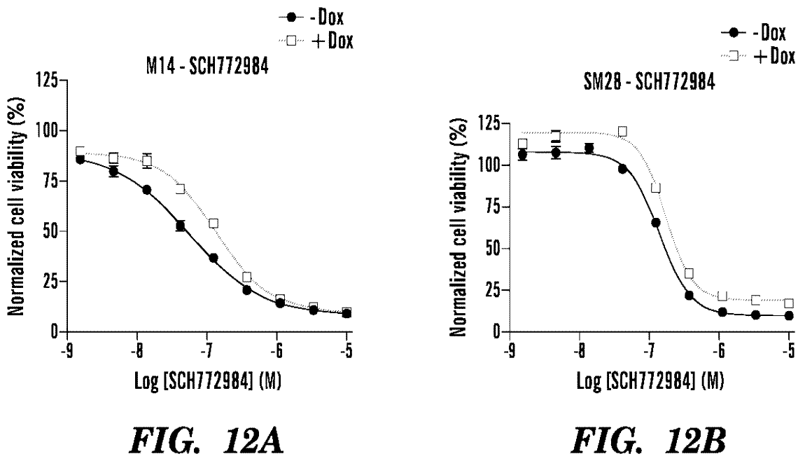

The inventors have identified loss-of-function mutations in Stag2 as well as decreased expression of Stag2/3 proteins in tumor samples from patients with acquired resistance to BRAFi and in BRAFi resistant melanoma cell lines. Knockdown of Stag2/3 decreased sensitivity to BRAF inhibitors in Val600Glu (V600E) BRAF-mutant melanoma cells and xenograft tumors. In addition, loss of Stag2/3 promotes the dimerization of BRAF and CRAF to reactivate MEK-ERK signaling and without wishing to be bound by theory, restore sensitivity to ERK inhibitors or mixed MEK/ERK inhibitors. Melanoma cells having a loss of Stag2 expression are sensitive to ERK inhibition (FIGS. 11A-11B, & 12A-12B), indicating that patients with Stag2 or Stag3 mutations should be treated with ERK inhibitors or mixed MEK/ERK inhibitors.

This technology is also contemplated herein to be applicable to subjects with other cancer types where mutations in the BRAF oncogene have been shown to play an important role. Such cancers include, but are not limited to papillary thyroid carcinoma, colorectal cancer, melanoma, and non-small-cell lung cancer. In addition, the methods described herein are useful to determine if a patient should discontinue BRAF inhibition therapy.

1. A method of treating cancer, the method comprising:
(i) receiving results of an assay measuring the activity and/or expression levels of STAG2 and/or STAG3 in a biological sample obtained from a subject having or suspected of having cancer, the results showing a decrease in the measured activity and/or expression levels of STAG2 and/or STAG3 compared to a reference; and
(ii) administering a PD-1 inhibitor, or a PD-L1 inhibitor, thereby treating the cancer in the subject.

2. The method of claim 1, wherein the results of the assay are the activity and/or expression levels of Stag2.

3. The method of claim 1, further comprising a step of selecting the subject having a cancer prior to receiving results of the assay, wherein the subject has a cancer comprising a mutation in BRAF, STAG2 and/or STAG3.

4. The method of claim 3, wherein the mutation in STAG2 is a loss-of-function mutation.

5. The method of claim 1, wherein the PD-1or PD-L1 inhibitor is nivolumab, pembrolizumab, pidilizumab, BMS-936559, or MPDL-3280A.

6. The method of claim 1, wherein the reference is the activity and/or expression of STAG2 and/or STAG3 in a population of subjects having a cancer known to be responsive to a BRAF inhibitor or having a cancer in a phase of responsiveness to a BRAF inhibitor.

7. The method of claim 1, wherein the cancer is selected from the group consisting of: bladder cancer, non-Hodgkin lymphoma, colorectal cancer, melanoma, papillary thyroid carcinoma, non-small-cell lung carcinoma, and adenocarcinoma of the lung.

8. The method of claim 1, wherein the received results of the assay are determined from an assay that comprises a step of contacting the biological sample with an antibody that specifically binds to STAG2 and/or STAG3.

9. The method of claim 3, wherein the mutation in BRAF, STAG2 and/or STAG3 is identified by a DNA sequencing method.

10. A method of treating a subject having a cancer that is resistant to or is developing resistance to a BRAF inhibitor, the method comprising:
(i) receiving the results of an assay measuring the activity and/or expression levels of STAG2 and/or STAG3 in a sample obtained from a subject being treated with a BRAF inhibitor, the results showing a decrease in the activity and/or expression levels compared to the activity and/or expression levels prior to the onset of treatment with a BRAF inhibitor, wherein the subject is determined to have a cancer that is resistant to or is developing resistance to a BRAF inhibitor, and
(ii) treating the subject with a PD-1 inhibitor or a PD-L1 inhibitor and optionally discontinuing treatment with the BRAF inhibitor, thereby treating the cancer in the subject.

11. The method of claim 10, wherein the PD-1or PD-L1 inhibitor is nivolumab, pembrolizumab, pidilizumab, BMS-936559, or MPDL-3280A.

12. The method of claim 10, wherein the cancer comprises a loss-of-function mutation in Stag2.

13. The method of claim 10, wherein the subject being treated with a BRAF inhibitor has a cancer selected from the group consisting of: bladder cancer, non-Hodgkin lymphoma, colorectal cancer, melanoma, papillary thyroid carcinoma, non-small-cell lung carcinoma, and adenocarcinoma of the lung.

14. The method of claim 10, wherein the results received from the assay are generated using an assay that comprises a step of contacting the biological sample with an antibody that specifically binds to STAG2 and/or STAG3.

15. The method of claim 12, wherein the loss-of-function mutation in STAG2 is identified by a DNA sequencing method.

16.-17. (canceled)

18. A method of treating cancer, the method comprising:
(i) measuring the activity and/or expression levels of STAG2 and/or STAG3 in a biological sample obtained from a subject having or suspected of having cancer, wherein a decrease in the measured activity and/or expression levels of STAG2 and/or STAG3 compared to a reference is detected; and
(ii) administering a PD-1 inhibitor, or a PD-L1 inhibitor, thereby treating the cancer in the subject.


SEQUENCE LISTING (27 sequences)

SEQ ID NO: 1 - DNA, 59 bp, synthetic construct
  misc_feature 1..59: note = Description of Artificial Sequence: Synthetic oligonucleotide
  source 1..59: mol_type = other DNA
ccgggcaagcagtcttcaggttaaactcgagtttaacctgaagactgcttgcttttttg

SEQ ID NO: 2 - DNA, 48 bp, synthetic construct
  misc_feature 1..48: note = Description of Artificial Sequence: Synthetic oligonucleotide
  source 1..48: mol_type = other DNA
cctaaggttaagtcgccctcgctcgagcgagggcgacttaaccttagg

SEQ ID NO: 3 - DNA, 19 bp, synthetic construct
  misc_feature 1..19: note = Description of Artificial Sequence: Synthetic oligonucleotide
  source 1..19: mol_type = other DNA
ttaatgctaagatttagtg

SEQ ID NO: 4 - DNA, 59 bp, synthetic construct
  misc_feature 1..59: note = Description of Artificial Sequence: Synthetic oligonucleotide
  source 1..59: mol_type = other DNA
ccggcgtgatttccttaggccacttctcgagaagtggcctaaggaaatcacgttttttg

SEQ ID NO: 5 - DNA, 23 bp, synthetic construct
  misc_feature 1..23: note = Description of Artificial Sequence: Synthetic primer
  source 1..23: mol_type = other DNA
gatagtggagattatccacttac

SEQ ID NO: 6 - DNA, 22 bp, synthetic construct
  misc_feature 1..22: note = Description of Artificial Sequence: Synthetic primer
  source 1..22: mol_type = other DNA
ctgccagggtgcttgtatgtcg

SEQ ID NO: 7 - DNA, 20 bp, synthetic construct
  misc_feature 1..20: note = Description of Artificial Sequence: Synthetic primer
  source 1..20: mol_type = other DNA
atgcctatgctcgcacaact

SEQ ID NO: 8 - DNA, 23 bp, synthetic construct
  misc_feature 1..23: note = Description of Artificial Sequence: Synthetic primer
  source 1..23: mol_type = other DNA
atactgagtccatttccctatgc

SEQ ID NO: 9 - DNA, 20 bp, synthetic construct
  misc_feature 1..20: note = Description of Artificial Sequence: Synthetic primer
  source 1..20: mol_type = other DNA
gaaccaaatggaaggtcaca

SEQ ID NO: 10 - DNA, 20 bp, synthetic construct
  misc_feature 1..20: note = Description of Artificial Sequence: Synthetic primer
  source 1..20: mol_type = other DNA
tgggtaaagatgatccgaca

SEQ ID NO: 11 - DNA, 20 bp, synthetic construct
  misc_feature 1..20: note = Description of Artificial Sequence: Synthetic primer
  source 1..20: mol_type = other DNA
ggctacatcctaggttcggt

SEQ ID NO: 12 - DNA, 18 bp, synthetic construct
  misc_feature 1..18: note = Description of Artificial Sequence: Synthetic primer
  source 1..18: mol_type = other DNA
caggatctgctccaggct

SEQ ID NO: 13 - DNA, 21 bp, synthetic construct
  misc_feature 1..21: note = Description of Artificial Sequence: Synthetic primer
  source 1..21: mol_type = other DNA
ctgcattgcgagaccaatcta

SEQ ID NO: 14 - DNA, 22 bp, synthetic construct
  misc_feature 1..22: note = Description of Artificial Sequence: Synthetic primer
  source 1..22: mol_type = other DNA
catccgagtctgttgcactatt

SEQ ID NO: 15 - DNA, 20 bp, synthetic construct
  misc_feature 1..20: note = Description of Artificial Sequence: Synthetic primer
  source 1..20: mol_type = other DNA
atcactgccacccagaagac

SEQ ID NO: 16 - DNA, 19 bp, synthetic construct
  misc_feature 1..19: note = Description of Artificial Sequence: Synthetic primer
  source 1..19: mol_type = other DNA
cagtgagcttcccgttcag

SEQ ID NO: 17 - DNA, 23 bp, synthetic construct
  misc_feature 1..23: note = Description of Artificial Sequence: Synthetic primer
  source 1..23: mol_type = other DNA
ctgaagactgtccgaaattatgc

SEQ ID NO: 18 - DNA, 22 bp, synthetic construct
  misc_feature 1..22: note = Description of Artificial Sequence: Synthetic primer
  source 1..22: mol_type = other DNA
ctgatttctccctactggtcac

SEQ ID NO: 19 - DNA, 21 bp, synthetic construct
  misc_feature 1..21: note = Description of Artificial Sequence: Synthetic primer
  source 1..21: mol_type = other DNA
ctccaacaggtttgctcttct

SEQ ID NO: 20 - DNA, 20 bp, synthetic construct
  misc_feature 1..20: note = Description of Artificial Sequence: Synthetic primer
  source 1..20: mol_type = other DNA
cccgagacgtttcagtcatt

SEQ ID NO: 21 - DNA, 20 bp, synthetic construct
  misc_feature 1..20: note = Description of Artificial Sequence: Synthetic primer
  source 1..20: mol_type = other DNA
ctggtctgtgctggccacgg

SEQ ID NO: 22 - DNA, 20 bp, synthetic construct
  misc_feature 1..20: note = Description of Artificial Sequence: Synthetic primer
  source 1..20: mol_type = other DNA
gcaccttggctggggctctg

SEQ ID NO: 23 - DNA, 13 bp, synthetic construct
  misc_feature 1..13: note = Description of Artificial Sequence: Synthetic oligonucleotide
  source 1..13: mol_type = other DNA
atatatgatgagt

SEQ ID NO: 24 - DNA, 13 bp, synthetic construct
  misc_feature 1..13: note = Description of Artificial Sequence: Synthetic oligonucleotide
  source 1..13: mol_type = other DNA
atatatratgagt

SEQ ID NO: 25 - DNA, 13 bp, synthetic construct
  misc_feature 1..13: note = Description of Artificial Sequence: Synthetic oligonucleotide
  source 1..13: mol_type = other DNA
aaacggaaagtgg

SEQ ID NO: 26 - DNA, 13 bp, synthetic construct
  misc_feature 1..13: note = Description of Artificial Sequence: Synthetic oligonucleotide
  source 1..13: mol_type = other DNA
aaacggtaagtgg

SEQ ID NO: 27 - DNA, 13 bp, synthetic construct
  misc_feature 1..13: note = Description of Artificial Sequence: Synthetic oligonucleotide
  source 1..13: mol_type = other DNA
gagcagatggtgt